SEQUENCE LISTING (50 sequences)

SEQ ID NO: 1 - protein, 17 aa, Solanum tuberosum
VSTSTYSGLLTNTYPPR

SEQ ID NO: 2 - protein, 12 aa, Solanum tuberosum
YIAVGNEVDPGR

SEQ ID NO: 3 - protein, 8 aa, Solanum tuberosum
AQNYANSR

SEQ ID NO: 4 - protein, 8 aa, Solanum tuberosum
GSGDFTGR

SEQ ID NO: 5 - protein, 13 aa, Solanum tuberosum
VTNTGTGTQETVR

SEQ ID NO: 6 - protein, 10 aa, Solanum tuberosum
AESIVQSTVR

SEQ ID NO: 7 - protein, 12 aa, Solanum tuberosum
WSPSAADSAAGR

SEQ ID NO: 8 - protein, 7 aa, Solanum tuberosum
ELGTVMR

SEQ ID NO: 9 - protein, 9 aa, Solanum tuberosum
IGQMTQIER

SEQ ID NO: 10 - protein, 12 aa, Solanum tuberosum
DIHGDILTPDSR

SEQ ID NO: 11 - protein, 10 aa, Solanum tuberosum
GQTWVINAPR

SEQ ID NO: 12 - protein, 9 aa, Solanum tuberosum
ALSGFSQQR

SEQ ID NO: 13 - protein, 13 aa, Solanum tuberosum
TVTNVGDATSSYK

SEQ ID NO: 14 - protein, 10 aa, Solanum tuberosum
LGSTPQTYTR

SEQ ID NO: 15 - protein, 13 aa, Solanum tuberosum
YNSDVGPSGTPVR

SEQ ID NO: 16 - protein, 12 aa, Solanum tuberosum
AYQEQVLSNSSK

SEQ ID NO: 17 - protein, 11 aa, Solanum tuberosum
YTGEGESDEAK

SEQ ID NO: 18 - protein, 8 aa, Solanum tuberosum
VDNVYGDR

SEQ ID NO: 19 - DNA, 21 bp, Artificial Sequence
  Primer
ttgaaggacaggaataagccc

SEQ ID NO: 20 - DNA, 23 bp, Artificial Sequence
  Primer
cccttagtcgtggattcttagca

SEQ ID NO: 21 - DNA, 21 bp, Artificial Sequence
  Primer
tgagaatgacgagcatctgtg

SEQ ID NO: 22 - DNA, 21 bp, Artificial Sequence
  Primer
ccccttagtcgtggattcttg

SEQ ID NO: 23 - DNA, 20 bp, Artificial Sequence
  Primer
tgtatcggaaagtggttggc

SEQ ID NO: 24 - DNA, 22 bp, Artificial Sequence
  Primer
ccaggtttctttggtgttccag

SEQ ID NO: 25 - DNA, 21 bp, Artificial Sequence
  Primer
ccttcagcaactctggaaaac

SEQ ID NO: 26 - DNA, 22 bp, Artificial Sequence
  Primer
caggtttctttggtgttccagt

SEQ ID NO: 27 - DNA, 22 bp, Artificial Sequence
  Primer
ccctgcagcaactttgaaaaat

SEQ ID NO: 28 - DNA, 22 bp, Artificial Sequence
  Primer
caggtttttttggtgttccagc

SEQ ID NO: 29 - DNA, 21 bp, Artificial Sequence
  Primer
cctgatcctcggtttgaagag

SEQ ID NO: 30 - DNA, 21 bp, Artificial Sequence
  Primer
gctcggaaaatcatgaccaac

SEQ ID NO: 31 - DNA, 21 bp, Artificial Sequence
  Primer
tgctgactatttccttgtggg

SEQ ID NO: 32 - DNA, 20 bp, Artificial Sequence
  Primer
tgctgaacttgtacgatccc

SEQ ID NO: 33 - DNA, 21 bp, Artificial Sequence
  Primer
aggaaactgaggctgtaaagg

SEQ ID NO: 34 - DNA, 22 bp, Artificial Sequence
  Primer
ctgtggtttatttggatgtggc

SEQ ID NO: 35 - DNA, 21 bp, Artificial Sequence
  Primer
agttctcattcctcgcatcag

SEQ ID NO: 36 - DNA, 21 bp, Artificial Sequence
  Primer
cccttcgtatgtgttctttgg

SEQ ID NO: 37 - DNA, 22 bp, Artificial Sequence
  Primer
tttcatcgtccaagcaataaca

SEQ ID NO: 38 - DNA, 22 bp, Artificial Sequence
  Primer
attcatcagcagcaaggatacc

SEQ ID NO: 39 - DNA, 20 bp, Artificial Sequence
  Primer
cagcgacacctcctaaatcc

SEQ ID NO: 40 - DNA, 20 bp, Artificial Sequence
  Primer
tccacgacagcaccaataac

SEQ ID NO: 41 - DNA, 21 bp, Artificial Sequence
  Primer
ggtatctccgtttccagagtg

SEQ ID NO: 42 - DNA, 21 bp, Artificial Sequence
  Primer
gcaaaggcttctaattctgcg

SEQ ID NO: 43 - DNA, 22 bp, Artificial Sequence
  Primer
ccgtactatcaagaggattggc

SEQ ID NO: 44 - DNA, 20 bp, Artificial Sequence
  Primer
atgttcctcaccaacgatcc

SEQ ID NO: 45 - DNA, 21 bp, Artificial Sequence
  Primer
tcaagcgggtaaagtatgtgg

SEQ ID NO: 46 - DNA, 21 bp, Artificial Sequence
  Primer
gacgttgtcctctccagttac

SEQ ID NO: 47 - DNA, 22 bp, Artificial Sequence
  Primer
gccaatataaacggtgaatgcc

SEQ ID NO: 48 - DNA, 21 bp, Artificial Sequence
  Primer
attgttgtcctccgaatgtgg

SEQ ID NO: 49 - DNA, 21 bp, Artificial Sequence
  Primer
attggaaacggatatgctcca

SEQ ID NO: 50 - DNA, 21 bp, Artificial Sequence
  Primer
tgacaggcgttcaggtaagga

(19) United States
(12) Patent Application Publication
(10) Pub. No.: US 2013/0172186 A1
(43) Pub. Date: Jul. 4, 2013

(54) PROTEINS RELATED TO THE SUPPRESSION OF PHYTOPHTHORA INFECTIONS IN MEMBERS OF THE SOLANACEAE FAMILY

(71) Applicants: Gefu Wang-Pruski, Bible Hill (CA); Sanghyun Lim, Quebec City (CA); Tudo Cristian Borza, Halifax (CA); Rex Andrew Schofield, Beaver Bank (CA)

(72) Inventors: Gefu Wang-Pruski, Bible Hill (CA); Sanghyun Lim, Quebec City (CA); Tudo Cristian Borza, Halifax (CA); Rex Andrew Schofield, Beaver Bank (CA)

(21) Appl. No.: 13/808,661

(22) PCT Filed: Jul. 7, 2011
(86) PCT No.: PCT/CA11/00770

§ 371 Date: Jan. 7, 2013

Related U.S. Application Data
(60) Provisional application No. 61/362,076, filed on Jul. 7, 2010.

(51) Int. Cl. C12N 15/113

(52) U.S. Cl. CPC C12N 15/113
USPC 504/196; 435/6.12; 435/23

(57) ABSTRACT

The present disclosure provides a method of suppressing oomycete infection, such as a Phytophthora infection, or triggering programmed cell death in Solanaceae plants comprising administering at least one modulator, wherein the at least one modulator modulates at least one of the genes listed in Table 1 or variants or homologs thereof. The present disclosure also provides compositions useful in suppressing oomycete infection, such as Phytophthora infection and triggering programmed cell death and screening assays for identifying substances useful in suppressing oomycete infection, such as Phytophthora infection and triggering programmed cell death.

### CROSS REFERENCE TO RELATED APPLICATIONS

This is a Patent Cooperation Treaty Application which claims the benefit of 35 U.S.C. 119 based on the priority of copending U.S. Provisional Patent Application No. 61/362,076 filed Jul. 7, 2010, which is incorporated by reference herein in its entirety.

### FIELD OF THE DISCLOSURE

The disclosure relates to proteins involved in the suppression of Phytophthora infections in members of the Solanaceae family and methods and compositions thereof.

### BACKGROUND OF THE DISCLOSURE

Late blight, caused by pathogen Phytophthora infestans (an oomycete), is the most severe disease of potatoes worldwide. Late blight control has been challenging since the disease overwinters as mycelium in seed tubers, on tubers in cull piles, and on un-harvested diseased tubers (volunteers) which survive the winter and become sources of inoculum. Once primary infection has occurred and the plant becomes infected, stem and leaf lesions can produce extremely large number of spores under favorable conditions. The spores can be airborne which spread the disease over great distances to other plants and other fields during wind and storm events.

Potato late blight caused by the oomycete Phytophthora infestans resulted in a 25% reduction of the population in Ireland in the 1840s. Another oomycete Plasmopara viticola caused great misery for grapevine agriculture in France in the 1880s. These two events led to the development of the discipline of plant pathology (Guest and Grant, 1991). Oomycetes, fungus-like but evolutionarily more closely related to brown algae, are economically important pathogens causing devastating diseases in a broad-spectrum of horticultural, ornamental, and forest species. Oomycetes of the genera Phythophthora spp. such as P. infestans, P. ramorum, P. cinnamomi, and P. palmivora causing diseases such as late blight, cocoa black pod and stem canker, chilli blight, dieback, and sudden oak death are widespread across North America, Europe, Australia, South Africa, Indonesia, Mexico, and Hawaii (Daniel and Guest, 2006; Balci et al., 2007; Cahill et al., 2008; King et al., 2010; McMahon et al., 2010).

Potato late blight is an important disease, causing enormous economic damage and over $3 billion annual losses worldwide due to costs of disease control and decreased production. To control P. infestans, the causal agent of late blight, the application of fungicides has been the management tool of choice (Fry, 2008). Bordeaux mixture, discovered by Millardet in 1885, was an effective fungicide for 90 years against grapevine downy mildew and late blight foliar diseases. However, it was not effective against soil-borne diseases, such as pink rot and leak tuber rot caused by P. erythroseptica and Pythium ultimum, respectively (Guest and Grant, 1991). The phenylamide-based systemic metalaxyl known as the most effective fungicide to control foliage disease and root-borne diseases simultaneously was introduced in the 1970s. It acts as an RNA synthesis inhibitor to prevent mycelial growth and haustoria formation of P. infestans (Matheron and Porchas, 2000). However, a strain of P. infestans resistant to metalaxyl was reported in Europe in 1981, in the US and Canada in the 1990s and thereafter worldwide, resulting in the loss of effective late blight disease control, especially tuber blight (Dowley and O'Sullivan, 1981; Goodwin et al., 1994). To date, chlorothalonil, a protectant fungicide, is the most heavily used to control potato late blight in the USA and Canada. However, concerns have been raised about its long term use and impacts on human health and the environment. Disease control is also restricted to foliar pathogens (Caux et al., 1996).

Metalaxyl is a protectant fungicide which was effective against the A1 mating type of late blight. Generally, two applications were sufficient if applied before onset of the disease occurs. However, during the summer of 1994, a new mating type of late blight known as A2, previously thought to exist only in Mexico, was found in the Atlantic region. This new mating type was very aggressive, appeared early in the season and worst of all, was found to be resistant to Ridomil, which was the main protective product on the market. Serious losses were incurred during that year. This crisis had prompted the establishment of many strategies for dealing with the disease since 1995. Still, current means of disease control is dependent only on few fungicides and they are all in high environmental risk category, causing soil and ground water contaminations.

Two major outbreaks of the disease in 2008 and 2009 led to huge losses in potato production. The growing conditions of Atlantic Canada are particularly amenable to the growth and spread of late blight meaning producers and processors are searching for products that safely control this disease. The current fungicides provide an effective control of the disease; however, due to the mixed occurrence of the pathogen strains, the risk of pathogen mutation is extremely high, meaning that existing fungicides may become ineffective in the near future. All these factors put potato production in a high risk position; therefore finding alternative solutions is an urgent need and will impact the industry significantly.

Pink rot is a fungus disease of potato tubers caused by Phytophthora erythroseptica which is found in most agricultural soils. It is characterized by wet rot and pink color of the cut surfaces of the tuber upon exposure to air. The disease is usually seen at harvest and can be spread during storage. It is one of the most damaging diseases in stored potatoes. The control of the disease is by foliage fungicide applications in fields and by tuber treatment before tubers are put into the storage facilities.

Phosphonate based chemicals have recently been used as a new fungicide for controlling oomycete pathogens by pretreatment before pathogen challenge (Daniel and Guest, 2006). The chemical was discovered by Rhone-Poulenc laboratories in France in the 1970s as a systemic antifungal agent. It is absorbed across membranes on plant foliage, stem or roots with great mobility and solubility. Therefore, phosphonates can be applied via root drench, stem injection, or foliar spray and are translocated via xylem as well as phloem. The name ‘phosphonate’ is commonly used to describe products made up of salts of phosphorous acid (H₃PO₃, PA). When phosphorous acid is dissolved in water, the strong acid form called phosphonic acid is produced. Alkali metal salts such as potassium or aluminum ions are added to make its pH neutral because the strong acid itself is harmful to plant tissues. The addition of potassium hydroxide forms the resulting solution called potassium phosphite or phosphorous acid and salts. Another resulting solution called fosetyl-Al is formed by the addition of aluminum ions. Phosphonate, potassium phosphite (or called as mono- and di-potassium salts of phosphorous acid) and fosetyl-Al fungicides are usually used in agricultural settings (Guest and Grant, 1991; FRAC Code list, 2009). Phosphonate fungicides are relatively inexpensive protectants with systemic properties to prevent foliar as well as root-borne diseases. In addition, phosphonates were classified as environmentally friendly biopesticides by the US Environmental Protection Agency (US-EPA) (Lobato et al., 2008; Mayton et al., 2008). Integrated crop management (ICM) promotes the reduction of the use of toxic fungicides which makes phosphonate an attractive alternative in ICM programs.

Confine™ is a phosphite (salt of phosphorous acid) based chemical product from The Agronomy Company of Canada. This product was registered as emergency registration in Canada in 2008 for suppression of late blight during tuber storage. It was registered again in 2009 for postharvest treatment of tubers for late blight suppression. In 2011, it was registered for foliage application for late blight disease prevention.

Phosphonate mode of action is complex and comprises a direct and indirect mode of action. The direct mode of action is triggered at higher concentrations, resulting in the inhibition of germination, zoospore production and mycelia growth of P. cinnamomi (Cohen and Coffey, 1986; Guest and Bompeix, 1990; Wilkinson et al., 2001). A change in levels of gene expression in P. cinnamomi after the addition of phosphonate in the cultured medium was reported (King et al., 2010). The expression of putative proteophosphoglycan gene was induced in culture with 5 μg/ml phosphonate. Other genes involved in cell wall biosynthesis, such as cellulose synthase and glycan synthase, were repressed in culture with 40 μg/ml phosphonate. At this 40 μg/ml concentration, about 70% of P. cinnamomi growth was inhibited 4 days later, suggesting the suppression of pathogen cell wall biosynthesis (Wong et al., 2009; King et al., 2010).

Phosphonate also triggers an indirect mode of action, resulting in the activation of plant defense responses (Jackson et al., 2000). The application of fosetyl-Al in tobacco plants led to the accumulation of phytoalexin and hypersensitive-like responses which halted the growth of P. nicotianae (Guest, 1984). Guest (1986) revealed that fosetyl-Al (100 μg/ml)-treated or phosphite (70 μg/ml)-treated tobacco seedlings following P. nicotianae challenge showed rapid cytoplasmic aggregation, host nuclei migration, and papillae apposition under microscopic observation. In the chemical-treated tobacco seedlings, the pathogen growth in 86% of observed instances was arrested at the local hypersensitive cell death sites in 24 h. In untreated seedlings with small papillae apposition, 83% of them succeeded with the intercellular penetration and sporangia were formed between 36-48 h. These defense responses were similar to those in genetically resistant seedlings after pathogen challenge (Guest, 1986). Tobacco cultivar (cv.) NC2326 with resistance against P. nicotianae conferred resistance by the rapid induction of the accumulation of sesquiterpenoid phytoalexins and an increase in the phenylalanine ammonia lyase (PAL) activity. Treatment of cv. NC2326 with mevinolin, an inhibitor of sesquiterpenoid biosynthesis, induced the susceptibility of cv. NC2326 resistant to P. nicotianae. However, the application of the inhibitor to fosetyl-Al-treated NC2326 did not induce complete susceptibility, suggesting that fosetyl-Al turns on more than one defense signaling pathway, including the sesquiterpenoid pathway. Treatment of tobacco cv. Hicks susceptible to P. nicotianae with fosetyl-Al conferred enhanced resistance in cv. Hicks, leading to the accumulation of lignin and ethylene in addition to an increase in the sesquiterpemoid phytoalexins and PAL activity (Nemestothy and Guest, 1990). In another study, phosphite-treated Eucalyptus marginata, a western Australian native tree, showed increased levels of defense enzymes, 4-coumarate coenzyme A ligase and cinnamyl alcohol dehydrogenase. Both are involved in the biosynthesis of lignin-related phenylpropanoids and phenolic compounds. These increases resulted in moderate resistance to P. cinnamomi, a soil-borne pathogen, in the treated plants (Jackson et al., 2000). The application of phosphite also reduced disease symptoms in the susceptible Lambertia Formosa, an Australian native plant species, following inoculation with P. cinnamomi, leading to an increase in superoxide release 8 h after pathogen inoculation and the increased PAL activity 24 h after the inoculation (Suddaby et al., 2008). In summary, phosphonate triggers an indirect mode of action, resulting in cell wall reinforcement such as lignin, phenolic compounds in tobacco-P. nicotianae interactions and in Australian native tree—P. cinnamomi interactions. Hypersensitive response (HR) symptoms were observed in tobacco-P. nicotianae interactions.

Phosphonate application may induce the primed state of plants. Several results suggested the possibility of primed plants following phosphonate applications. First, the treatment of an Australian grass tree Xanthorrhoea australis with phosphonate did not produce any anatomical responses before P. cinnamomi challenge. After pathogen infection, the susceptible tree displayed an increase in the biosynthesis of phenolic compounds in leaves, leading to enhanced resistance against the pathogen (Daniel et al., 2005). In another study, the activity of phytoalexins and phenols in tuber slices from fosetyl-Al-treated potato plants were increased at low levels before P. infestans challenge. After the pathogen challenge, the accumulation of phytoalexins and phenols was increased approximately tenfold and fivefold, respectively, in tubers from treated plants compared to the control tubers after the infection (Andreu et al., 2006).

The effect of phosphonate in potato-P. infestans interactions has also been studied. The application of fosetyl-Al to potato cv. Kennebec foliage induces an increase of phenol and phytoalexin contents in the foliage. Increased levels of β-1,3-glucanase and aspartic protease by fosetyl-Al application were observed in tubers obtained from fosetyl-Al-treated potato plants. The tubers showed enhanced resistance to late blight, suggesting that fosetyl-Al is an inducer involved in systemic acquired resistance (SAR) in the tubers (Andreu et al., 2006). However, the molecular mechanisms involved have not been documented (Andreu et al., 2006; Lobato et al., 2008). Daniel and Guest (2006) used the Arabidopsis-P. palmivora pathosystem to identify defense responses activated by phosphonate. The authors identified the involvement of cytoplasmic aggregation, nuclear migration, localized cell death, the release of superoxide, and the accumulation of phenolic compounds in phosphite-treated seedlings (Daniel and Guest, 2006). They also pointed out that “meanwhile, the signaling cascade activated in the phosphonate-treated plant remains enigmatic” (Daniel and Guest, 2006). Similar statements were made by Andreu et al. (2006) “Mechanisms that induce and allow systemic acquired resistance (SAR) persistence are largely unknown”. The latest report published by Wang-Pruski et al. (2010) demonstrated a three-year field trial using the pretreatment of potato plants with Confine™, a recently registered fungicide that is composed of mono- and di-potassium salts of phosphorous acid (PA). The outcome confirmed the consistent protection provided by PA against late blight in potatoes.

Plants, when exposed to pathogens, activate their innate immune system. This system can be turned on when receptors in plants recognize pathogens' conserved regions and/or pathogen effector proteins upon infection. The presence of the corresponding receptors in the plants determines their resistance against the pathogen. Soon after the recognition of the pathogen by the receptors, these plants activate defense responses in undamaged remote cells by signalling transduction pathways, resulting in resistance to the pathogen in these tissues. For plants susceptible to a pathogen, induced resistance (IR) called adaptive immunity can be triggered by pre-treatments of inducing agents, like vaccination, leading to the activation of plant defense responses. IR confers enhanced resistance in susceptible plants against a broad-spectrum of pathogens. Many inducers work in dose- and/or time-dependent manners in plants, resulting in the induction of a unique physiological state in plants called priming. The primed plants display enhanced resistance with minimum side effects.

Pathogen-associated molecular patterns (PAMP)-triggered defense responses include plant cell wall reinforcement, oxidative burst, the accumulation of antimicrobial metabolites, the increase of pathogenesis-related (PR) proteins, and changes in levels of plant hormones (Pieterse et al., 2009).

HR (hypersensitive response) is a type of programmed cell death that leads to rapid host cell death at local infection sites in order to inhibit the growth of either biotrophs or hemibiotrophs (Mur et al., 2008). It is the major mechanism in plants for resistance to the pathogen (Rooney at al., 2005).

Soon after activating innate immune responses at local infection sites, plants transmit a signal(s) from the infection sites to the uninfected distant cells in order to protect undamaged tissues. The mobile signal induces the accumulation of salicylic acid (SA) and the induced SA increases reactive oxygen species (ROS) levels in the healthy cells. Ultimately, defense response in undamaged cells is boosted to resistance, called systemic acquired resistance (SAR) (Ryals et al., 1994). SAR induced by SA application is the best known mechanism associated with IR (Oostendorp et al., 2001). SAR can be induced by SA or the SA analogue benzo(1,2,3)-thiadiazole-7-carbothioic acid S-methyl ester (BTH). BTH application led to the inhibition of catalase and ascorbate peroxidase and an increase in the ROS production (Wendehenne et al., 1998). SAR in plants features a long-lasting resistance from a few weeks to a few months to cope with a secondary infection (Kuc, 1987; Durrant and Dong, 2004). SA leads to an increase in the H₂O₂ concentration by inhibiting the scavenging enzymes ascorbate peroxidase and catalase. The concentration of SA can also be increased by the high concentration of H₂O₂ (Glazebrook, 2005).

Similar to SAR triggered by pathogens upon infection at local infection sites in plant innate immunity, pre-treatment with many inducing agents also triggers SAR. However, molecular mechanisms of induced resistance (IR) elicited by many inducers remains to be investigated since the effects and importance of IR in susceptible plants are known.

### SUMMARY OF THE DISCLOSURE

The present inventors are the first to identify a series of proteins that are regulated by the chemical phosphorous acid, which may be useful in suppressing Phytophthora infection and in triggering programmed cell death in plants. In particular, the inventors have identified 72 proteins that are upregulated and 31 proteins that are downregulated after phosphorous acid treatment through proteomic profiling. The inventors have also identified 13 functionally related proteins that are either upregulated or downregulated after phosphorous acid treatment.

Since Phytophthora belongs to oomycetes, the methods, compositions and assays disclosed herein are also useful for suppressing infections of other oomycetes, such as Plasmopara and Pythium including Pythium ultimum). Accordingly, the present disclosure provides a method of suppressing an infection caused by an oomycete in a member of the Solanaceae family comprising administering at least one modulator to a Solanaceae plant or cell, wherein the at least one modulator modulates at least one of the genes listed in Table 1 or of genes encoding functionally related proteins.

In one embodiment, the present disclosure provides a method of suppressing a Phytophthora infection in a member of the Solanaceae family comprising administering at least one modulator to a Solanaceae plant or cell, wherein the at least one modulator modulates at least one of the genes listed in Table 1 or of genes encoding functionally related proteins. In one embodiment, the Phytophthora infection is delayed by at least 0.5 weeks, at least 1 week, at least 1.5 weeks or at least 2 weeks. In another embodiment, the Solanaceae plant is a potato plant. In an embodiment, the potato is a Russet Burbank or Shepody variety.

In one embodiment, the Phytophthora infection is Phytophthora infestans. In another embodiment, the Phytophthora infection is Phytophthora erythroseptica, Phytophthora ramorum, Phytophthora cinnamomi, Phytophthora nicotianae and Phytophthora palmivora.

In an embodiment, the method of suppressing a Phytophthora Infestans infection suppresses late blight disease. In another embodiment, the method of suppressing a Phytophthora erythroseptica infection suppresses pink rot disease. In yet another embodiment, the method of suppressing Phytophthora ramorum infection suppresses sudden oak death. In a further embodiment, the method of suppressing a Phytophthora cinnamomi infection suppresses dieback or root rot/stem canker.

The disclosure also provides a method of triggering programmed cell death in a Solanaceae plant or cell comprising administering at least one modulator to a Solanaceae plant or cell, wherein the at least one modulator modulates at least one of the genes listed in Table 1 or of genes encoding functionally related proteins.

In one embodiment, the at least one modulator comprises an activator of at least one of the genes listed in Table 1A and/or an inhibitor of at least one of the genes listed in Table 1B, or of genes encoding functionally related proteins. In another embodiment, the at least one modulator comprises an activator of at least 2, 5, 10, 20, 30, 40, 50, 60, 70, 72 or 84 of the genes listed in Table 1A or of genes encoding functionally related proteins and/or comprises an inhibitor of at least 2, 5, 10, 20, 30, 31 or 32 of the genes listed in Table 1B or of genes encoding functionally related proteins. In another embodiment, at least 2, 5, 10, 20, 30, 40, 50, 60, 70, 72 or 84 activators of the genes listed in Table 1A or genes encoding functionally related proteins are administered and/or at least 2, 5, 10, 20, or 30, 31 or 32 inhibitors of the genes listed in Table 1B or of genes encoding functionally related proteins are administered. In one embodiment, the modulator comprises an activator of at least one of the genes listed in Table 1A identified through proteomic profiling (i.e. the first 72 genes listed in the Table 1A). In another embodiment, the modulator comprises an inhibitor of at least one of the genes listed in Table 1B identified through proteomic filing (i.e. the first 31 genes listed in Table 1B).

In addition to proteomic filing, the present inventors identified 13 other genes that are PA-responsive from MRM and qPCR. Accordingly, in yet another embodiment, the at least one modulator modulates at least one of the genes listed in Table 7 or 10. These genes are also listed in Tables 1A and 1B as new genes.

In one embodiment, the activator comprises an isolated nucleic acid molecule of at least one of the genes listed in Table 1A or variants or homologs thereof. In an embodiment, the nucleic acid molecule comprises a recombinant expression vector. In another embodiment, the recombinant expression vector is contained in a host cell. In yet another embodiment, the activator comprises a protein or variant or homolog thereof encoded by at least one of the genes listed in Table 1A or a variant or homolog thereof.

In an embodiment, the inhibitor comprises an antisense RNA of at least one of the genes listed in Table 1B or variants or homologs thereof or a siRNA molecule or shRNA molecule that inhibits expression of at least one of the genes listed in Table 1B or variants or homologs thereof or an aptamer that inhibits at least one of the proteins encoded by the genes listed in Table 1B or variants or homologs thereof. In another embodiment, the inhibitor comprises an antibody or antibody fragment against a protein encoded by at least one of the genes listed in Table 1B or variants or homologs thereof.

Also provided herein are compositions comprising the modulators disclosed herein and screening assays for identifying substances useful in suppressing Phytophthora infection or triggering cell death and diagnostic methods for determining the effectiveness of Phytophthora infection treatment.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows field trials showing reduced infection by phosphorous acid in the detached leaves in 2007 and in field plants in 2008 and 2009.

FIG. 2 shows a graphical analysis of the functions of the 103 PA-regulated proteins identified through proteomic profiling. The abundance of these proteins showed statistically significant difference (Fold Change>1.4) in their comparisons.

FIG. 3 shows a graphical analysis of the functions of the 72 up-regulated (FIG. 3A) and 31 down-regulated proteins (FIG. 3B) identified through proteomic profiling. The abundance of these proteins showed statistically significant difference in their comparisons.

FIG. 4 shows one group of proteins in defense category.

FIG. 5 shows day 4, 5 and 6 leaf samples control and treated with PA, indicating the differences in infection areas.

FIG. 6 shows day 4, 5 and 6 leaf samples control and treated with PA, showing the enlarged leaves the differences the in infection areas.

FIG. 7 shows day 4, 5 and 6 leaf samples control and treated with PA, showing the enlarged leaves the differences the in infection areas.

FIG. 8 shows control day 5 and 6 leaf samples and DAB staining. The diseased areas are wide spread.

FIG. 9 shows PA-treated day 5 and day 6 leaf samples and DAB staining. The diseased areas are limited in small areas.

FIG. 10 shows control and PA-treated day 5 and day 6 leaf samples and DAB staining.

FIG. 11 shows tuber samples 8 days after infected by late blight pathogen. These Russet Burbank tubers were tested in March 2010. They were harvested from the 2009 field trials treated by PA or untreated as controls. The PA treated tubers showed less infection than the control tubers.

FIG. 12 shows the fold changes of the 15 proteins validated by MRM. The diagrams are generated based on the data from Table 8. Threshold for significant changes of relative abundance are 1.4 folds for up-regulated proteins and 0.75 folds for down-regulated proteins. Two proteins, TC164121 and TC163226, are significantly down-regulated; all other 13 proteins are significantly up-regulated.

FIG. 13 shows the progression of late blight (P. infestans A2 US8) infection on potato plants treated with 1% Confine (one application) and on untreated plants.

FIG. 14 shows genes analyzed by qRT-PCR whose products (proteins) were identified using proteomics and which have roles in plant defense mechanisms. Analysis by qRT-PCR at different intervals after Confine application on plants confirmed a general trend of gene up-regulation. a) Basic pathogenesis-related protein 1, b) osmotin, c) beta 1,3 glucanase (members of class I and II) and, d) beta 1,3 glucanase (class II family). Change that is statistically significant: * p<0.1; ** p<0.05.

FIG. 15 shows genes analyzed by qRT-PCR whose products (proteins) were identified using proteomics and which are involved in various metabolic pathways and energy production. Analysis by qRT-PCR at different intervals after Confine application on plants confirmed a general trend of gene down-regulation in two of the three genes analyzed. a) Alpha glucan phosphorylase type H, b) Alpha glucan phosphorylase type L1 and, c) sucrose synthase 2. Change that is statistically significant: * p<0.1; ** p<0.05.

FIG. 16 shows genes analyzed by qRT-PCR whose products (proteins) perform cellular functions related to those identified using proteomics. a) Beta 1,3 glucanase (class I), b) Alpha glucan phosphorylase type L2, c) sucrose synthase 4, d) 1,4 alpha glucan branching enzyme, e) fructose bisphosphate aldolase, f) mitochondrial ATPase, subunit b, g) chloroplastidial ATPase, subunit a, and h) chloroplastidial ATPase, subunit b. Change that is statistically significant: * p<0.1; ** p<0.05. a-b are primarily related to plant defense mechanisms, c-e products in starch and sugar metabolism and, f-h in energy generation.

FIG. 17 shows a schematic of changes in subcellular structures in cells showing HR related cell death symptoms. Figure was adapted from Coll et al., 2011.

FIG. 18 shows cell death was not observed on the infected site in control leaves by light microscopy (LM) and scanning electron microscopy (SEM).

FIG. 19 shows cell death was observed on the infected site in PA treated leaves by LM and SEM.

FIGS. 20A, 20B, and 20C show detection of HR cell death using transmission electron microscopy (TEM).

FIG. 21 shows detection of HR cell death by callose deposition. Control 5 dpi (FIG. 21A): No cell death was seen and no localized callose deposition was observed. PA 5 dpi (FIG. 21B): Cell death was seen and localized callose deposition was observed.

FIG. 22 shows sporangial count of potato slices after 5 to 7 days (d5, d6, d7) infection with Phytophthora infestans. Data points and error bars represent the means of three slices with the standard error of the mean. Treatment effects were analyzed within each cultivar and separate comparisons were made each day. For Shepody (SH) and Prospect (P), the treatments were significantly different at each day (p-value 0.01), except for d6 Prospect where there was no difference. For Russet Burbank (RB), there were no significant treatment effects on individual days; although, when averaged over the three days there was a significant treatment effect (p-value 0.05). C—Control untreated tubers; T—Confine treated tubers.

FIG. 23 shows area of infected potato slices was estimated as percent gray area after 5 to 7 (d5, d6, d7) days infection with Phytophthora infestans. Data points and error bars represent the means of at least three slices with the standard error of the mean. Treatment effects were analyzed within each cultivar and separate comparisons were made each day. For Shepody (SH) and Prospect (P), the treatments were significantly different at each day (p-value 0.01), except for d5 and d6 Prospect (p-value 0.05). For Russet Burbank (RB), there were no significant treatment effects on individual days; although, when averaged over the three days there was a significant treatment effect (p-value 0.05). C—Control untreated tubers; T—Confine treated tubers.

FIG. 24 shows Day 7 (D7) photos of infected potato slices. SH—Shepody; RB—Russet Burbank; P—Prospect.

FIG. 25 shows area of infected potato slices that turned brown due to damage after 5 to 7 days (d5, d6, d7) of infection with Phytophthora infestans. Data points and error bars represent the means of at least five slices with the standard error of the mean. Treatment effects were analyzed within each cultivar and separate comparisons were made each day. For each cultivar, the treatments were different at each day (p-value 0.001), except for d7 Shepody (SH) and Prospect (P) (p-value 0.05). RB—Russet Burbank. C—Control untreated tubers; T—Confine treated tubers.

FIG. 26 shows area of infected potato slices covered with white mycelia/sporangia after 5 to 7 days (d5, d6, d7) infection with Phytophthora infestans. Data points and error bars represent the means of at least five slices with the standard error of the mean. Treatment effects were analyzed within each cultivar and separate comparisons were made each day. For each cultivar, the treatments were different at each day (p-value 0.001). SH—Shepody, P—Prospect, RB—Russet Burbank. C—Control untreated tubers; T—Confine treated tubers.

FIG. 27 shows growth chamber grown potato plants (var. Shepody) treated with either 1% Confine or water (Control) and infected 10 days after the treatments. Top panel: Days after infection from Confine treated plants. Bottom panel: Days after infection from water treated plants (Control).

FIG. 28 shows close view of the leaves from the plants shown in FIG. 27. Confine treated plants typically show small brown infected spots on leaves (left), but the infection does not spread for the observation period of over 30 days. The leaves from control plants (right) show the mass production of sporangia 7 days after infection.

FIG. 29 shows boxplots showing infection severity of days 4, 5, 6, 7 and 10. X-axis shows the samples from the control and the four treatments. Y-axis shows the infection severity from 0% (the least) to 100% (the most).

FIG. 30 shows the Confine application on potato slices influences late blight growth in a concentration-dependent manner.

### DETAILED DESCRIPTION OF THE DISCLOSURE

The present inventors evaluated whether Confine™ (phosphorous acid or “PA”) effectively suppressed late blight in foliage during the growing period and, identified its mode of actions. The three-year field trials conducted at Cavendish Farms in PEI strongly suggested that PA application delays the disease occurrence in fields for at least two weeks. This delay not only significantly increased the yield, but also provided the harvested tubers higher resistance to late blight in storage.

The present inventors identified 103 proteins that are up or down regulated in potato leaf tissues after PA treatment, which are herein referred to as PA-responsive proteins. Forty-five out of the 72 up-regulated proteins are involved in plant defense mechanisms. Four from the 31 down-regulated proteins are also involved in plant defense mechanisms. With further experimentation, an additional 13 functionally related proteins were also identified as being PA-responsive. The behaviour of some of the proteins changed after pathogen infection. The functions of these proteins are involved in hypersensitive response (HR) and SA signalling [a salicylic acid (SA)-mediated defense responses as an activator of systemic acquired resistance (SAR)]. The overall results of the responses are to inhibit fungal growth by degrading fungal cell walls and to inhibit the fungal spread by triggering programmed plant cell death. Without wishing to be bound by any theory, it is suggested that phosphorous acid activated HR in plants. When the pathogen attacked, rapid cell death process was induced, that resulted in the formation of a zone of dead cells around the site of infection. This overall effect reduced the production of the sporangia, therefore, delaying the spread of the disease in the field.

Since Phytophthora belongs to oomycetes, the methods, compositions and assays disclosed herein are also useful for suppressing infections of other oomycetes, such as Plasmopara and Pythium including Pythium ultimum). Accordingly, the present disclosure provides a method of suppressing an infection caused by an oomycete in a member of the Solanaceae family comprising administering at least one modulator to a Solanaceae plant or cell, wherein the at least one modulator modulates at least one of the genes listed in Table 1 or of genes encoding functionally related proteins. In one embodiment, the oomycete is Phytophthora, Plasmopara or Pythium.

The present disclosure also provides a method of suppressing Phytophthora infection in a member of the Solanaceae family comprising administering at least one modulator to a Solanaceae plant or cell, wherein the at least one modulator modulates at least one of the genes listed in Table 1 or variants or homologs thereof or of genes encoding functionally related proteins.

In one embodiment, the at least one modulator comprises an activator of at least one of the genes listed in Table 1A or variants or homologs thereof or of genes encoding functionally related proteins and/or an inhibitor of at least one of the genes listed in Table 1B or variants or homologs thereof or of genes encoding functionally related proteins. In another embodiment, the at least one modulator modulates at least one of the genes listed in Tables 7 and/or 10 or variants or homologs thereof.

The phrase “genes encoding functionally related proteins” as used herein refers to genes that encode proteins that have similar functions in the plant, including genes encoding proteins of the following categories: i) proteins primarily involved in defense mechanisms; ii) proteins functioning in metabolic pathways and energy production; iii) proteins related to synthesis/protein turnover; and iv) proteins that are involved in signal transduction.

The term “member of the Solanaceae family”, also called nightshades, comprise more than 3000 species. The Solanaceae are the third most important plant taxon economically and the most valuable in terms of vegetable crops, and are the most variable of crops species in terms of agricultural utility, as it includes the tuber-bearing potato, a number of fruit-bearing vegetables (tomato, eggplant, peppers), ornamental plants (petunias, Nicotiana), plants with edible leaves (Solanum aethiopicum, S. macrocarpon) and medicinal plants (eg. Datura, Capsicum). In one embodiment, the Solanaceae plant is a potato. In another embodiment, the Solanaceae plant is a tomato.

In one embodiment, the Phytophthora infection is Phytophthora infestans. In another embodiment, the Phytophthora infection is Phytophthora erythroseptica, Phytophthora ramorum, Phytophthora cinnamomi, Phytophthora nicotianae and Phytophthora palmivora.

In an embodiment, the method of suppressing a Phytophthora Infestans infection suppresses late blight disease. In another embodiment, the method of suppressing a Phytophthora erythroseptica infection suppresses pink rot disease. In yet another embodiment, the method of suppressing a Phytophthora ramorum infection suppresses sudden oak death. In a further embodiment, the method of suppressing Phytophthora cinnamomi infection suppresses dieback or root rot/stem canker.

The term “late blight disease” as used herein refers to a disease caused by the pathogen Phytophthora infestans.

The term “pink rot disease” as used herein refers to a disease caused by the pathogen Phytophthora erythroseptica.

The phrase “suppressing a Phytophthora infection” as used herein refers to delaying the onset of disease, for example, by at least 0.5 weeks, at least 1 week, at least 1.5 weeks or at least 2 weeks.

Also disclosed herein is a method of triggering programmed cell death in a Solanaceae plant or cell comprising administering at least one modulator as disclosed herein to a Solanaceae plant or cell. In one embodiment, the Solanaceae plant or cell is infected with a Phytophthora infection. In another embodiment, the at least one modulator modulates at least one of the genes listed in Table 1 or variants or homologs thereof or of genes encoding functionally related proteins, optionally at least one of the genes listed in Tables 7 and/or 10 or variants or homologs thereof. In one embodiment, the Solanaceae plant is a potato, such as a potato infected with Phytophthora infestans or Phytophthora erythroseptica. In another embodiment, the Solanaceae plant is a tomato, such as a tomato infected with Phytophthora infestans. Other plants include fruit-bearing vegetables (eggplant, peppers), ornamental plants (petunias, Nicotiana), plants with edible leaves (Solanum aethiopicum, S. macrocarpon) and medicinal plants (eg. Datura, Capsicum).

The term modulator refers to a substance that is an activator or an inhibitor with the proviso that the modulator is not phosphorous acid.

In one embodiment, the modulator is a protein or nucleic acid molecule involved in plant defense mechanisms. In another embodiment, the modulator is a protein or nucleic acid molecule involved in hypersensitive response or salicylic acid signaling. In yet another embodiment, the modulator is a protein or nucleic acid molecule involved in energy/metabolism, protein synthesis, signaling/transcription or protein destination.

The term “activator” as used herein includes any substance that increases the expression or activity of at least one of the genes listed in Table 1A or of genes encoding functionally related proteins and includes, without limitation, providing additional nucleic acid molecules of the genes listed in Table 1A or the encoded proteins or variants, homologs or fragments thereof, small molecule activators, antibodies (and fragments thereof), and other substances that can activate expression or activity. In an embodiment, the activator activates at least 2, 5, 10, 20, 30, 40, 50, 60, 70, 72 or 84 of the genes listed in Table 1A or variants or homologs thereof or of genes encoding functionally related proteins. In one embodiment, the activator activates at least one of the genes listed as upregulated in Tables 9 and/or 11 or variants or homologs thereof.

The term “inhibitor” as used herein includes any substance that decreases the expression or activity of at least one of the genes listed in Table 1B or variants or homologs thereof or of genes encoding functionally related proteins and includes, without limitation, providing antisense nucleic acid molecules of said genes, siRNAs or shRNAs of said genes, proteins, antibodies (and fragments thereof), small molecule inhibitors and other substances directed at expression or activity. In an embodiment, the inhibitor inhibits at least 2, 5, 10, 20, 30, 31 or 32 of the genes listed in Table 1B or variants or homologs thereof of genes encoding functionally related proteins. In one embodiment, the inhibitor inhibits at least one of the genes listed as downregulated in Tables 9 and/or 11 or variants or homologs thereof.

The term “potato” as used herein refers to any plant tuber from the nightshade or potato family, and includes, all potato varieties, including without limitation, Russet Burbank and Shepody varieties.

The term “administering a modulator” includes both the administration of the modulator as well as the administration of a nucleic acid sequence encoding the modulator to a potato onto a cell in vitro (or ex vivo) or in vivo. The term “administering” also includes the administration of a cell that expresses the modulator as well as insertion of a recombinant gene into the plant.

The term “a cell” includes a single cell as well as a plurality or population of cells. Administering to a cell includes administering in vitro (or ex vivo) as well as in vivo.

In another embodiment, at least 2, 5, 10, 20, 30, 40, 50, 60, 70, 72 or 84 activators of the genes listed in Table 1A or of genes encoding functionally related proteins are administered and/or at least 2, 5, 10, 20, 30, 31 or 32 inhibitors of the genes listed in Table 1B or of genes encoding functionally related proteins are administered. In yet another embodiment, 1-10, 11-20, 21-30, 31-40, 41-50, 51-60, 61-70, 71-80, 81-90, 92-100, 101, 102, 103 or 116 modulators, each modulator modulating at least one of the genes listed in Table 1 or of genes encoding functionally related proteins, are administered.

In one embodiment, the activator comprises an isolated nucleic acid molecule of at least one of the genes listed in Table 1A or variants or homologs thereof, optionally at least one of the genes listed in Tables 9 and/or 11 as up-regulated

The term “nucleic acid molecule” is intended to include unmodified DNA or RNA or modified DNA or RNA. For example, the nucleic acid molecules or polynucleotides of the disclosure can be composed of single- and double stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “isolated and/or purified” as used herein refers to a nucleic acid or amino acid substantially free of cellular material or culture medium when produced by recombinant DNA techniques, or chemical precursors, or other chemicals when chemically synthesized. An “isolated and/or purified” nucleic acid is also substantially free of sequences which naturally flank the nucleic acid (i.e. sequences located at the 5′ and 3′ ends of the nucleic acid) from which the nucleic acid is derived.

In another embodiment, the activator comprises at least one isolated protein or variant thereof encoded by at least one of the genes listed in Table 1A or a variant or homolog thereof or genes encoding functionally related proteins, optionally at least one of the genes listed in Tables 9 and/or 11 as being upregulated or a variant or homolog thereof. The term “amino acid” includes all of the naturally occurring amino acids as well as modified amino acids.

The term “isolated protein” refers to a polypeptide substantially free of cellular material or culture medium when produced by recombinant DNA techniques, or chemical precursors or other chemicals when chemically synthesized.

The term “variant” as used herein includes, without limitation, modifications, substitutions, including without limitation, conservative substitutions, additions, derivatives, analogs, fragments or chemical equivalents of the nucleic acid or amino acid sequences disclosed herein that perform substantially the same function in substantially the same way. Variants would have the same function of being useful to suppress Phytophthora infection or triggering programmed cell death.

The term “fragment” as used herein means a portion of a polypeptide that contains at least 10%, 20%, 30%, 40%, 50%, 60%, 70%, 80%, 90%, 95%, or more of the entire length of the reference polypeptide.

The term “homolog” means those amino acid or nucleic acid sequences which have slight or inconsequential sequence variations from the sequences of the genes listed in Table 1, i.e., the sequences function in substantially the same manner. The variations may be attributable to local mutations or structural modifications. In one embodiment, a homolog is the related gene from a different organism. Sequences having substantial homology include nucleic acid sequences having at least 65%, at least 85%, or 90-95% identity with the sequences of the genes listed in Table 1.

The term “analog” means an amino acid or nucleic acid sequence which has been modified wherein the modification does not alter the utility of the sequence (e.g. as a late blight disease suppressor) as described herein. The modified sequence or analog may have improved properties over the sequences shown in Table 1. One example of a nucleic acid modification to prepare an analog is to replace one of the naturally occurring bases (i.e. adenine, guanine, cytosine or thymidine) of the sequence with a modified base such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecules. For example, the nucleic acid sequences may contain phosphorothioates, phosphotriesters, methyl phosphonates, and phosphorodithioates.

A further example of an analog of a nucleic acid molecule of the disclosure is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complementary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506). The analogs may also contain groups such as reporter groups, a group for improving the pharmacokinetic or pharmacodynamic properties of nucleic acid sequence.

A “conservative amino acid substitution” as used herein, is one in which one amino acid residue is replaced with another amino acid residue without abolishing the desired function or activity of the modulators disclosed herein. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. Conserved amino acid substitutions involve replacing one or more amino acids of the polypeptides of the disclosure with amino acids of similar charge, size, and/or hydrophobicity characteristics. When only conserved substitutions are made the resulting molecule should be functionally equivalent. Changes which result in production of a chemically equivalent or chemically similar amino acid sequence are included within the scope of the disclosure. If the modulators of the present disclosure are made using recombinant DNA technology, conservative substituted variants of the modulators may be made by using polypeptide engineering techniques such as site directed mutagenesis, which are well known in the art for substitution of amino acids. For example, a hydrophobic residue, such as glycine can be substituted for another hydrophobic residue such as alanine. An alanine residue may be substituted with a more hydrophobic residue such as leucine, valine or isoleucine. A negatively charged amino acid such as aspartic acid may be substituted for glutamic acid. A positively charged amino acid such as lysine may be substituted for another positively charged amino acid such as arginine. The phrase “conservative substitution” also includes the use of a chemically derivatized residue in place of a non-derivatized residue provided that such polypeptide displays the requisite activity.

The term “derivative” refers to a peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. A derivative of a polypeptide also optionally includes polypeptides comprising forms of amino acids that are oxidized.

Variants also include peptides with amino acid sequences that are substantially or essentially identical to the amino acid sequences encoded by the genes listed in Table 1 or nucleic acid molecules with nucleic acid sequences that are substantially or essentially identical to the nucleic acid sequences of the genes listed in Table 1.

The term “substantially identical” or “essentially identical” as used herein means an amino acid or nucleic acid sequence that, when optimally aligned, for example using the methods described herein, share at least 75%, 80%, 85%, 90%, 95%, 96%, 97%, 98%, 99%, or 100% sequence identity with a second amino acid or nucleic acid sequence.

The term “sequence identity” as used herein refers to the percentage of sequence identity between two polypeptide and/or nucleotide sequences.

To determine the percent identity of two sequences, the sequences are aligned for optimal comparison purposes (e.g., gaps can be introduced in the sequence of a first amino acid or nucleic acid sequence for optimal alignment with a second amino acid or nucleic acid sequence). The amino acid or nucleotide residues at corresponding amino acid or nucleotide positions are then compared. When a position in the first sequence is occupied by the same amino acid residue or nucleotide as the corresponding position in the second sequence, then the molecules are identical at that position. The percent identity between the two sequences, is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. An optional, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecule of the present disclosure. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another optional, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The percentage of identity between two polypeptide sequences, the amino acid sequences of such two sequences are aligned, for example using the Clustal W algorithm (Thompson, J D, Higgins D G, Gibson T J, 1994, Nucleic Acids Res. 22(22): 4673-4680.), together with BLOSUM 62 scoring matrix (Henikoff S. and Henikoff J. G., 1992, Proc. Natl. Acad. Sci. USA 89: 10915-10919) and a gap opening penalty of 10 and gap extension penalty of 0.1, so that the highest order match is obtained between two sequences wherein at least 50% of the total length of one of the sequences is involved in the alignment.

Other methods that may be used to align sequences are the alignment method of Needleman and Wunsch (Needleman and Wunsch. J. Mol. Biol., 1970, 48:443), as revised by Smith and Waterman (Smith and Waterman. Adv. Appl. Math. 1981, 2:482) so that the highest order match is obtained between the two sequences and the number of identical amino acids is determined between the two sequences. Other methods to calculate the percentage identity between two amino acid sequences are generally art recognized and include, for example, those described by Carillo and Lipton (Carillo and Lipton SIAM J. Applied Math. 1988, 48:1073) and those described in Computational Molecular Biology (Computational Molecular Biology, Lesk, e.d. Oxford University Press, New York, 1988, Biocomputing: Informatics and Genomics Projects). Generally, computer programs will be employed for such calculations.

The disclosure further encompasses nucleic acid molecules that differ from any of the nucleic acid molecules disclosed herein in codon sequences due to the degeneracy of the genetic code.

The modulators described herein may also contain or be used to obtain or design “peptide mimetics”. For example, a peptide mimetic may be made to mimic the function of an activator or inhibitor. “Peptide mimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features. Peptide mimetics also include molecules incorporating peptides into larger molecules with other functional elements (e.g., as described in WO 99/25044). Peptide mimetics also include peptoids, oligopeptoids (Simon et al (1972) Proc. Natl. Acad, Sci USA 89:9367) and peptide libraries containing peptides of a designed length representing all possible sequences of amino acids corresponding to a modulator peptide.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of the secondary structures of the proteins described herein. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

In yet another embodiment, the inhibitor comprises an antisense RNA of at least one of the genes listed in Table 1b or variants or homologs thereof or genes encoding functionally related proteins, optionally at least one of the genes listed in Tables 9 and/or 11 as being downregulated or variants or homologs thereof. In another embodiment, the inhibitor is a siRNA molecule or shRNA molecule that inhibits expression of at least one of the genes listed in Table 1B or variants or homologs thereof or of genes encoding functionally related proteins, optionally at least one of the genes listed in Tables 9 and/or 11 as being downregulated or variants or homologs thereof. In a further embodiment, the inhibitor is an aptamer that inhibits at least one of the proteins encoded by the genes listed in Table 1B or variant or homologs thereof or by genes encoding functionally related proteins, optionally at least one of the proteins encoded by the genes listed in Tables 9 and/or 11 as being downregulated or variants or homologs thereof.

The term “antisense nucleic acid” as used herein means a nucleotide sequence that is complementary to its target e.g. a transcription product of the genes listed in Table 1b. The nucleic acid can comprise DNA, RNA or a chemical analog, that binds to the messenger RNA produced by the target gene. Binding of the antisense nucleic acid prevents translation and thereby inhibits or reduces target protein expression. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules or shRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

Aptamers are short strands of nucleic acids that can adopt highly specific 3-dimensional conformations. Aptamers can exhibit high binding affinity and specificity to a target molecule. These properties allow such molecules to specifically inhibit the functional activity of proteins and are included as agents that inhibit at least one of the genes listed in Table 1b or variants or homologs thereof or of genes encoding functionally related proteins.

In another embodiment, the inhibitor comprises an antibody against a protein encoded by at least one of the genes listed in Table 1B or variants or homologs thereof or of genes encoding functionally related proteins, optionally a protein encoded by at least one of the genes listed in Tables 9 and/or 11 as being downregulated or variants or homologs thereof. In an embodiment, the antibody is specific to at least one of the genes listed in Table 1B or variants or homologs thereof. In one embodiment, the antibody is a blocking antibody.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Antibodies to such proteins may be prepared using techniques known in the art such as those described by Kohler and Milstein, Nature 256, 495 (1975) and in U.S. Pat. Nos. RE 32,011; 4,902,614; 4,543,439; and 4,411,993, which are incorporated herein by reference. (See also Monoclonal Antibodies, Hybridomas: A New Dimension in Biological Analyses, Plenum Press, Kennett, McKearn, and Bechtol (eds.), 1980, and Antibodies; A Laboratory Manual, Harlow and Lane (eds.), Cold Spring Harbor Laboratory Press, 1988, which are also incorporated herein by reference). Within the context of the present disclosure, antibodies are understood to include monoclonal antibodies, polyclonal antibodies, antibody fragments (e.g., Fab, and F(ab′)₂) and recombinantly produced binding partners.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Continuous cultures of fused cells secreting antibody of predefined specificity. Nature 256:495-497, 1975) as well as other techniques such as the human B-cell hybridoma technique (Kozbor, D, and Roder, J: The production of monoclonal antibodies from human lymphocytes. Immunology Today 4:3 72-79, 1983), the EBV-hybridoma technique to produce human monoclonal antibodies (Cole et al. Monoclonal Antibodies in Cancer Therapy (1985) Allen R. Bliss, Inc., pages 77-96) and screening of combinatorial antibody libraries (Huse, W, Sastry, L, Iverson, S, Kang, A, Alting-Mees, M, Burton, D, Benkovic, S, and Lerner, R: Generation of a large combinatorial library of the immunoglobulin repertoire in phage lambda. Science 246:4935 1275-1282, 1989). Hybridoma cells can be screened immunochemically for production of antibodies specifically reactive with the protein or fragment thereof and the monoclonal antibodies can be isolated. Therefore, the disclosure also contemplates hybridoma cells secreting monoclonal antibodies with specificity for at least one of the proteins encoded by the genes listed in Table 1b or a variant or fragment thereof.

For producing recombinant antibodies (see generally Huston et al, 1991; Johnson and Bird, 1991; Mernaugh and Mernaugh, 1995), messenger RNAs from antibody producing B-lymphocytes of animals, or hybridoma are reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries. Antibody cDNA can also be inserted into a plant cell, such as a potato cell and used to produce a transgenic plant line, such as a transgenic potato line.

Specific antibodies, or antibody fragments, reactive against one of the proteins encoded by the genes listed in Table 1b or by genes encoding functionally related proteins or a variant or fragment thereof may also be generated by screening expression libraries encoding immunoglobulin genes, or portions thereof, expressed in bacteria with peptides produced from the nucleic acid molecules encoding the protein of interest or a variant or fragment thereof. For example, complete Fab fragments, VH regions and FV regions can be expressed in bacteria using phage expression libraries (See for example Ward et al. (Binding activities of a repertoire of single immunoglobulin variable domains secreted from Escherichia coli. Nature 348:544-546, 1989), Huse et al., supra and McCafferty et al (Phage antibodies: filamentous phage displaying antibody variable domains. Nature 348:552-555, 1989)).

Antibodies may also be prepared using DNA immunization. For example, an expression vector containing a nucleic acid encoding the protein of interest or a variant or fragment thereof may be injected into a suitable animal such as mouse. The protein will therefore be expressed in vivo and antibodies will be induced. The antibodies can be isolated and prepared as described above.

The proteins described above (including truncations, analogs, etc.) may be prepared using recombinant DNA methods. These proteins may be purified and/or isolated to various degrees using techniques known in the art. Accordingly, the disclosure also includes expression vectors comprising a nucleic acid sequence disclosed herein. Possible expression vectors include but are not limited to cosmids, plasmids, artificial chromosomes, viral vectors or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the disclosure and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The disclosure therefore contemplates a composition comprising a recombinant expression vector of the disclosure containing a nucleic acid molecule of the disclosure, or a fragment thereof, and the necessary regulatory sequences for the transcription and translation of the inserted protein-sequence.

Suitable-regulatory sequences may be derived from a variety of sources, including plant, bacterial, fungal, viral, mammalian, or insect genes (for example, see the regulatory sequences described in Goeddel, Gene Expression Technology Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector. It will also be appreciated that the necessary regulatory sequences may be supplied by the sequences listed in Table 1 and/or their flanking regions.

The recombinant expression vectors of the disclosure may also contain a selectable marker gene which facilitates the selection of host cells transformed or transfected with a recombinant molecule of the disclosure. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin optionally IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the disclosure and in particular to determine the effect of a mutation on expression and phenotype. It will be appreciated that selectable markers can be introduced on a separate vector from the nucleic acid of interest.

The recombinant expression vectors may also contain genes which encode a moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The term “transformed host cell” is intended to include cells that are capable of being transformed or transfected with a recombinant expression vector of the disclosure. The terms “transduced”, “transformed with”, “transfected with”, “transformation” and “transfection” are intended to encompass introduction of nucleic acid (e.g. a vector or naked RNA or DNA) into a cell by one of many possible techniques known in the art. Prokaryotic cells can be transformed with nucleic acid by, for example, electroporation or calcium-chloride mediated transformation. For example, nucleic acid can be introduced into plant or mammalian cells via conventional techniques such as calcium phosphate or calcium chloride co-precipitation, DEAE-dextran mediated transfection, lipofectin, electroporation, microinjection, RNA transfer, DNA transfer, artificial chromosomes, viral vectors and any emerging gene transfer technologies. For example, genes can typically be transferred into the potato genome by Agrobacterium mediated T-DNA transfer methods known in the art. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of eukaryotic host cells and prokaryotic cells. For example, the proteins of the disclosure may be expressed in yeast cells, insect cells, transgenic plant cells, eukaryotic or prokaryotic cell-free expression systems, or mammalian cells. Other suitable host cells can be found in Goeddel, Gene Expression Technology: Methods in Enzymology 185, Academic Press, San Diego, Calif. (1991). In addition, the proteins of the disclosure may be expressed in prokaryotic cells, such as Escherichia coli (Zhang et al., Science 303(5656): 371-3 (2004)) or in prokaryotic expression platforms such as Gram positive and lactic acid bacteria, including without limitation, Streptococcus gordonii, Lactococcus lactis and Lactobacillus spp.

The proteins disclosed herein may also be prepared by chemical synthesis using techniques well known in the chemistry of proteins such as solid phase synthesis (Merrifield, J. Am. Chem. Assoc. 85:2149-2154 (1964); Frische et al., J. Pept. Sci. 2(4): 212-22 (1996)) or synthesis in homogenous solution (Houbenweyl, Methods of Organic Chemistry, ed. E. Wansch, Vol. 15 I and II, Thieme, Stuttgart (1987)).

N-terminal or C-terminal fusion proteins comprising the proteins disclosed herein with other molecules, such as proteins may be prepared by fusing, through recombinant techniques. The resultant fusion proteins contain a modulator fused to the selected protein or marker protein as described herein. The recombinant protein may also be conjugated to other proteins by known techniques. For example, the proteins may be coupled using heterobifunctional thiol-containing linkers as described in WO 90/10457, N-succinimidyl-3-(2-pyridyldithio-proprionate) or N-succinimidyl-5 thioacetate. Examples of proteins which may be used to prepare fusion proteins or conjugates include cell binding proteins such as immunoglobulins, hormones, growth factors, lectins, insulin, low density lipoprotein, glucagon, endorphins, transferrin, bombesin, asialoglycoprotein glutathione-S-transferase (GST), hemagglutinin (HA), and truncated myc.

Also disclosed herein is a composition comprising at least one of the modulators disclosed herein. In one embodiment, there is provided a composition comprising at least one activator disclosed herein and/or at least one inhibitor disclosed herein in admixture with a suitable carrier. In one embodiment, there is provided a composition comprising at least one activator of at least one of the genes listed in Table 1A or variants or homologs thereof or of genes encoding functionally related proteins and/or at least one inhibitor of at least one of the genes listed in Table 1B or variants or homologs thereof or of genes encoding functionally related proteins, in admixture with a suitable carrier. In one embodiment, the at least one activator is a sense nucleic acid of a gene listed in Table 1A or a variant or homolog thereof. In another embodiment, the at least one inhibitor is an antisense nucleic acid of a gene listed in Table 1B or a variant or homolog thereof. In yet another embodiment, the at least one activator is a protein encoded by a gene listed in Table 1A or a variant or homolog thereof. In a further embodiment, the at least one inhibitor is an antibody against said protein. In another embodiment, the composition comprises at least one modulator of at least one of the genes listed in Tables 7 and/or 10 or variants or homologs thereof.

In one embodiment, the composition comprises an activator of at least 2, 5, 10, 20, 30, 40, 50, 60, 70, 72 or 84 of the genes listed in Table 1A or variants or homologs thereof or of genes encoding functionally related proteins. In another embodiment, the composition comprises an inhibitor of at least 2, 5, 10, 20, 30, 31 or 32 of the genes listed in Table 1B or variants or homologs thereof or of genes encoding functionally related proteins.

In one embodiment, the composition comprises at least 2, 5, 10, 20, 30, 40, 50, 60, 70, 72 or 84 activators of the genes listed in Table 1A or variants or homologs thereof or of genes encoding functionally related proteins and/or at least 2, 5, 10, 20, 30, 31 or 32 inhibitors of the genes listed in Table 1B or variants or homologs thereof or of genes encoding functionally related proteins.

Suitable carriers for administration to a Solanaceae plant are known in the art, including without limitation, water. In an embodiment, the composition is sprayed on the leaves of the plants by the field spraying methods described in the Examples.

### Screening Assays

Also disclosed herein is a screening assay for identifying a substance useful in suppressing an oomycete infection, such as Phytophthora infection, in Solanaceae plants comprising (a) administering a test substance to a Solanaceae cell or plant; and (b) determining the expression level of at least one of the genes listed in Table 1A and/or Table 1B or variants or homologs thereof compared to a control; wherein an increase in the expression level of the at least one gene listed in Table 1A or variant or homolog thereof or a decrease in the expression level of the at least one gene listed in Table 1B or variant or homolog thereof indicates that the test substance is useful in suppressing the oomycete infection, such as a Phytophthora infection. In an embodiment, the expression level of at least 2, 5, 10, 20, 30, 40, 50, 60, 70, 72 or 84 of the genes listed in Table 1A or variants or homologs thereof and/or at least 2, 5, 10, 20, 30, 31 or 32 of the genes listed in Table 1B or variants or homologs thereof are determined. In yet another embodiment, the expression level of at least one of the first 72 genes listed in Table 1A are determined and/or the expression level of at least one of the first 31 genes listed in Table 1B are determined. In another embodiment, the expression level of at least one of the genes listed in Tables 7 and/or 10 is determined. In one embodiment, the oomycete infection is Phytophthora infection.

The test substance can be any compound which one wishes to test including, but not limited to, proteins, peptides, nucleic acids (including RNA, DNA, antisense oligonucleotide, peptide nucleic acids), carbohydrates, organic compounds, small molecules, natural products, library extracts and other samples that one wishes to test for activity.

The term “expression level” of a gene as used herein refers to the measurable quantity of a gene product produced by the gene, wherein the gene product can be a transcriptional product or a translated transcriptional product. Accordingly, the expression level can pertain to a nucleic acid gene product such as RNA or cDNA or a polypeptide gene product. The expression level is derived from a plant sample or cell and/or a control sample, and can for example be detected de novo or correspond to a previous determination. The expression level can be determined or measured for example, using microarray methods, PCR methods, and/or antibody based methods, as is known to a person of skill in the art. RNA can also be directly quantitated using for example direct RNA sequencing or can be quantitated from cDNA pools. The MRM method described in this document can also be applied for this purpose.

Accordingly in one embodiment, determining the expression level comprises determining the level of RNA encoded by at least one of the genes listed in Table 1 or a variant or homolog thereof.

Determination of a level of RNA encoded by a gene in a sample may be effected in any one of various ways routinely practiced in the art. For example, the level of RNA encoded by a gene a sample may be determined via any one of various methods based on quantitative polynucleotide amplification which are routinely employed in the art for determining a level of RNA encoded by a gene in a sample.

Alternatively, the level of RNA encoded by a gene may be determined via any one of various methods based on quantitative polynucleotide hybridization to a probe which are routinely employed in the art for determining a level of RNA encoded by a gene in a sample.

In another embodiment, determining the expression level comprises determining the level of protein encoded by at least one of the genes listed in Table 1 or a variant or homolog thereof, for example, by assaying for binding of an antibody that recognizes a protein encoded by at least one of the genes listed in Table 1.

Expression levels can also be determined by methods described in the Examples.

The term “control” as used herein refers to a cell, cell sample and/or a numerical value or range corresponding to a gene expression level in a cell or cell sample, wherein the cell or cell sample has not been exposed to the test substance. Where the control is a numerical value or range, the numerical value or range is a predetermined value or range that corresponds to a level of gene expression or range of levels of the genes in the unexposed sample.

The phrase “increase or decrease in expression level” as used herein refers to a significant increase or decrease compared to control, for example, wherein the level of significance is P<0.05, P<0.01, P<0.005 or P<0.001.

In yet another embodiment, the disclosure provides a method of determining whether a treatment is effective for suppressing an oomycete infection, such as Phytophthora infection, comprising determining the expression level of at least one of the genes listed in Table 1A and/or Table 1B in a Solanaceae plant or cell that has been treated for oomycete infection, such as Phytophthora infection, compared to a control in the absence of treatment; wherein an increase in the expression level of the at least one gene listed in Table 1A or a decrease in the expression level of the at least one gene listed in Table 1B indicates that the treatment is effective in suppressing the oomycete infection, such as Phytophthora infection. In an embodiment, the expression level of at least 2, 5, 10, 20, 30, 40, 50, 60, 70, 72 or 84 of the genes listed in Table 1A and/or at least 2, 5, 10, 20, 30, 31 or 32 of the genes listed in Table 1B are determined. In yet another embodiment, the expression level of at least one of the first 72 genes listed in Table 1A are determined and/or the expression level of at least one of the first 31 genes listed in Table 1B are determined. In another embodiment, the expression level of at least one of the genes listed in Tables 7 and/or 10 is determined. In one embodiment, the oomycete infection is Phytophthora infection.

The above disclosure generally describes the present disclosure. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### 1. Field Trials

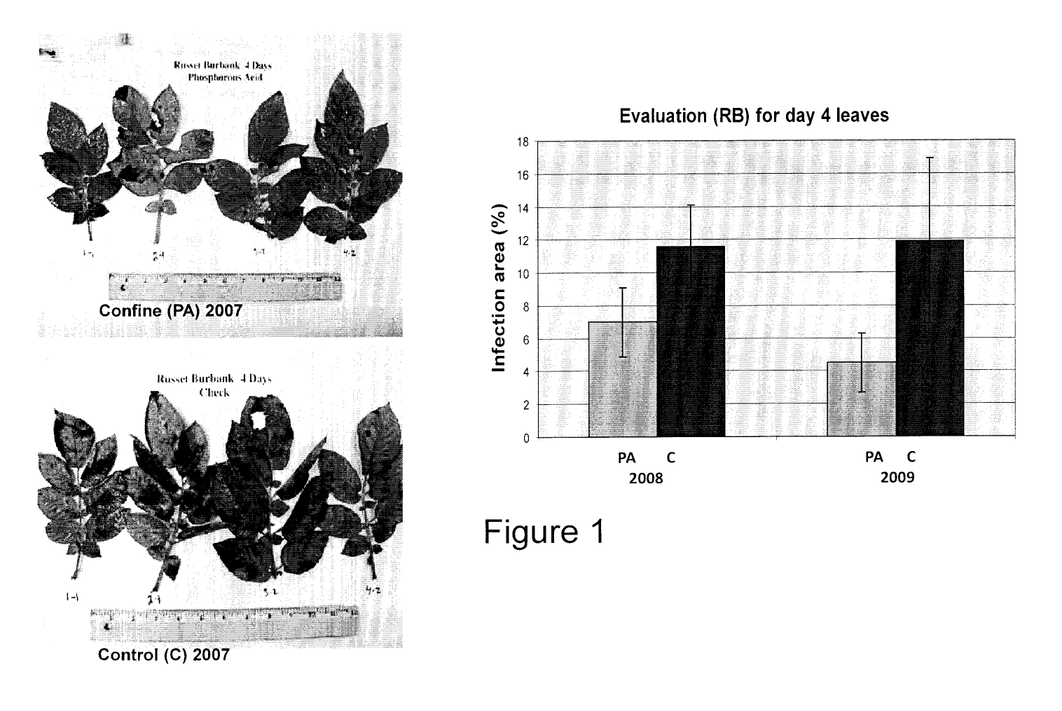

Three-year field trials (2007, 2008, 2009) have been completed. Total four treatments of control (water), PA, Bravo (a current commercial fungicide), PA combined with Bravo were made in two processing varieties ‘Russet Burbank’ and ‘Shepody’, each replicated four times. The trials were located at Cavendish Farms' research plots at Summerside, PEI following standard field production protocols. Field data, such as disease symptoms and yield, were collected each year. Leaf samples from the treated and non-treated field plots were harvested in August each season and challenged by the late blight pathogen using detached leaf assay. They were used for infection evaluation and for protein profiling. The results from the three seasons consistently demonstrated that phosphorous acid alone or in combination with Bravo significantly affected the suppression of the spread of the disease symptom on the infected leaves. There was no natural occurring late blight in the field in 2007; however, fields were naturally infected by late blight both in 2008 and 2009. In these two seasons, potato plants from PA treated plots showed at least two weeks delay in disease symptom development. The tuber yields from the treated plots were significantly higher than the non-treated plots (FIG. 1). Moreover, when applied together with Bravo, only half of the Bravo was required to maintain the effectiveness of disease control.

### 2. Quantitative Proteomic Profiling

Protein profiles have been generated from all samples before and after the pathogen infection using detached field leaf samples. The comparative proteomic analysis was carried out at the proteomic facility located at Institute for Marine Biosciences, National Research Council (NRC-IMB), Halifax. Both cell wall and cytosolic fractions were extracted and quantitated. Based on the methods generated, over 70% of the total proteins could be reproduced in biological sample replicates. This percentage was considered to be at a very high reproducibility. Total four groups of samples were undergone proteomic profiling. Each group was labeled with a distinct florescent dye namely 114, 115, 116, and 117. Dye 114 is for 0 day control without pathogen inoculation; dye 115 for 0 day PA without pathogen inoculation; dye 116 is for control 4-day post pathogen inoculation; dye 117 is for PA 4-day post pathogen inoculation. Comparative proteomic analysis using potato genome index databases have been completed and protein function annotations were also completed. Total 103 proteins, showed significant up- or down-regulations in PA-treated samples in comparison to the control samples, and were identified based on three biological replications. These proteins are referred to as PA-responsive proteins. Among the 103 proteins, 72 of them are up-regulated and 31 are down-regulated. The list of these proteins can be found in Table 1A and Table 1B. Table 1 also includes an additional 13 genes identified in Example 2.

### 3. Plant Defense Functional Pathway Analysis

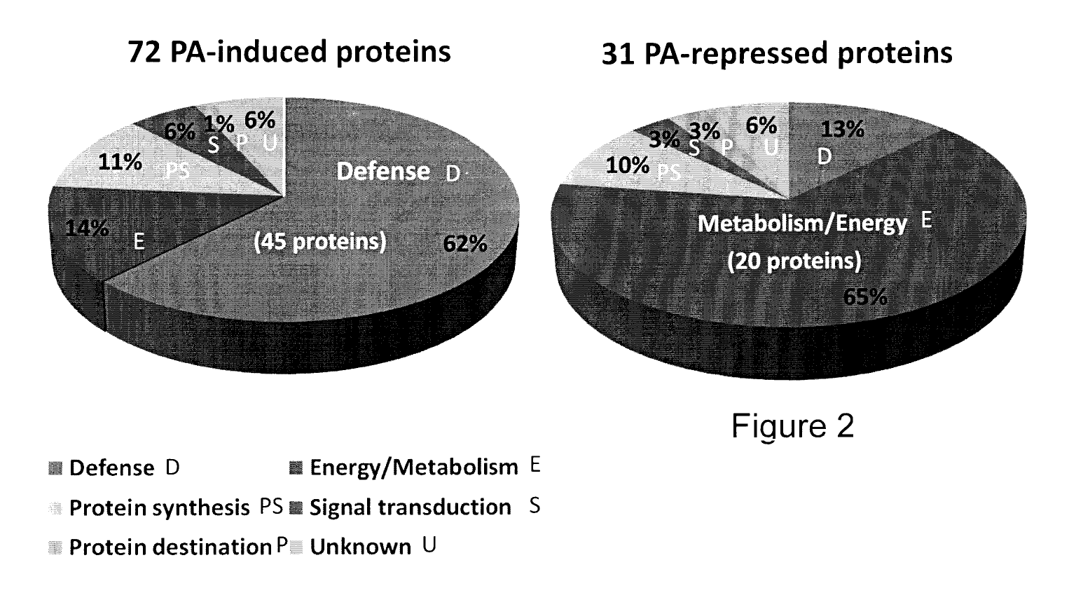

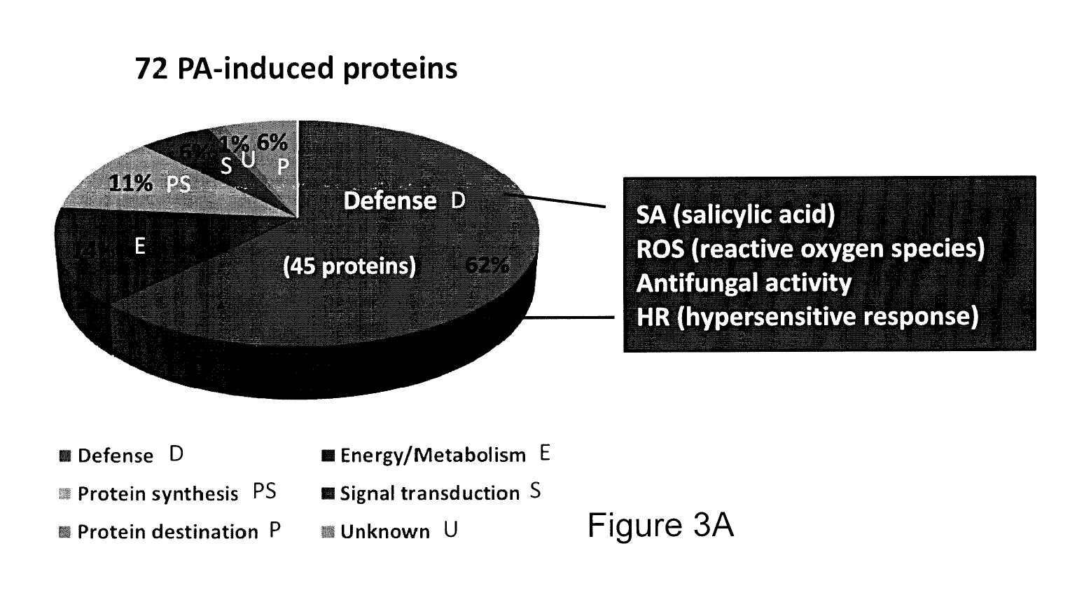

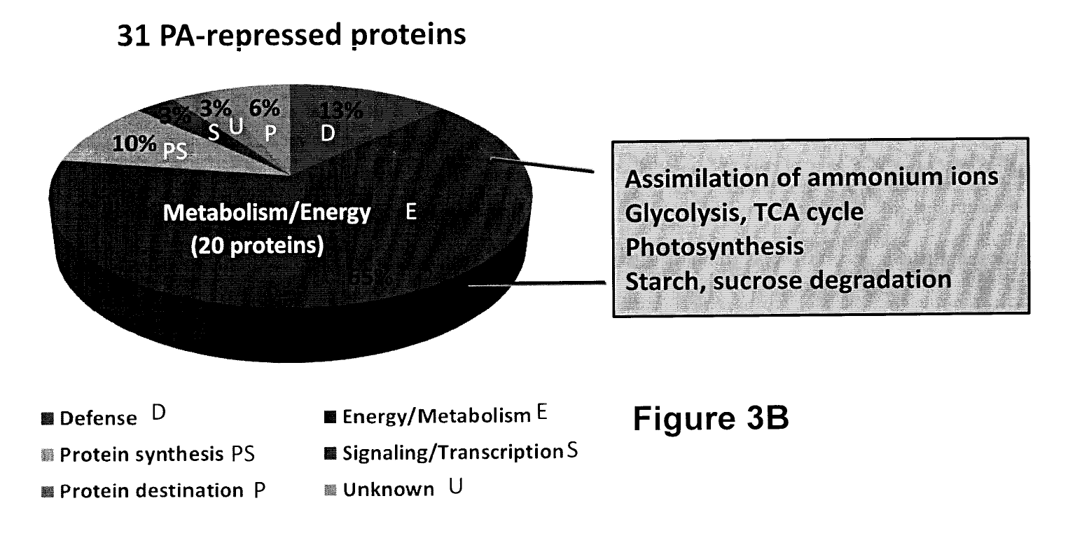

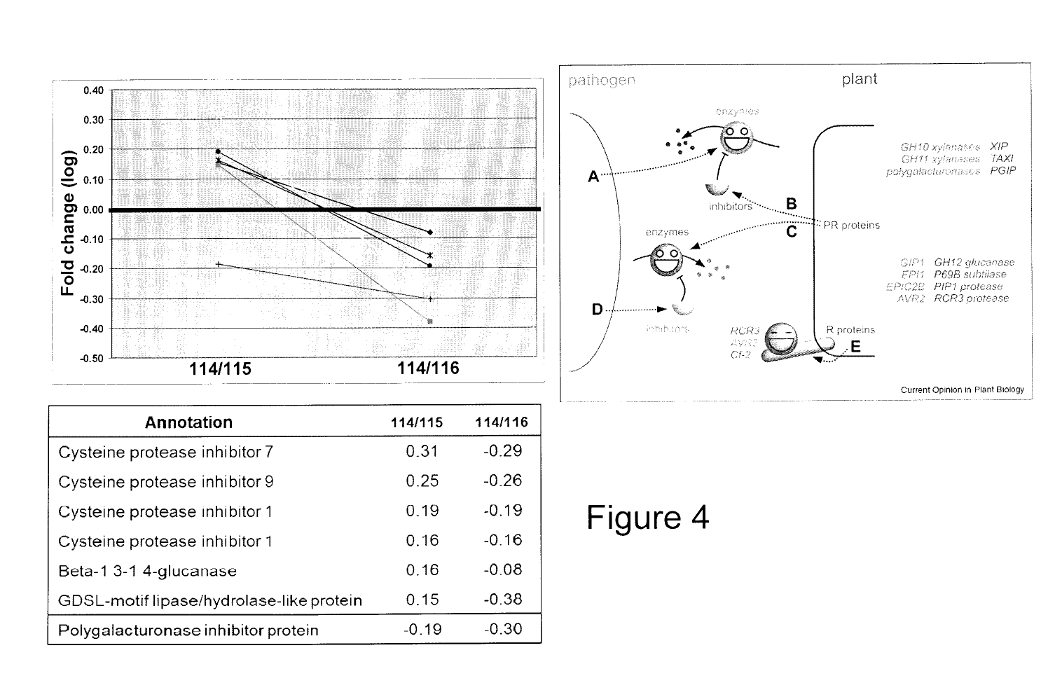

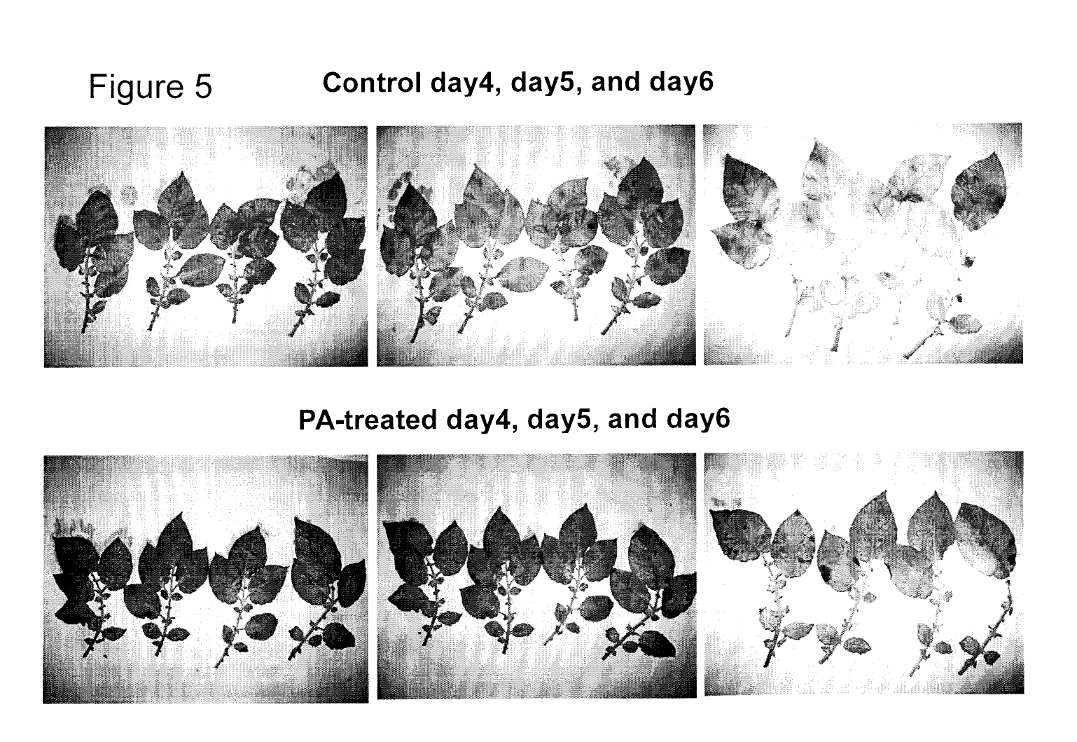

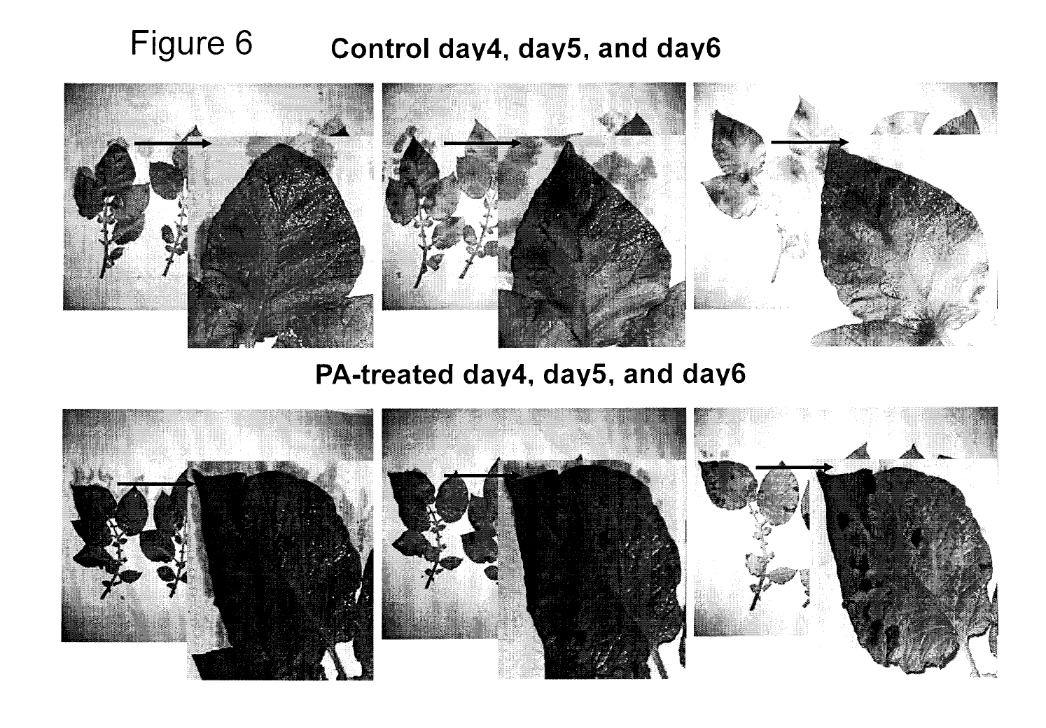

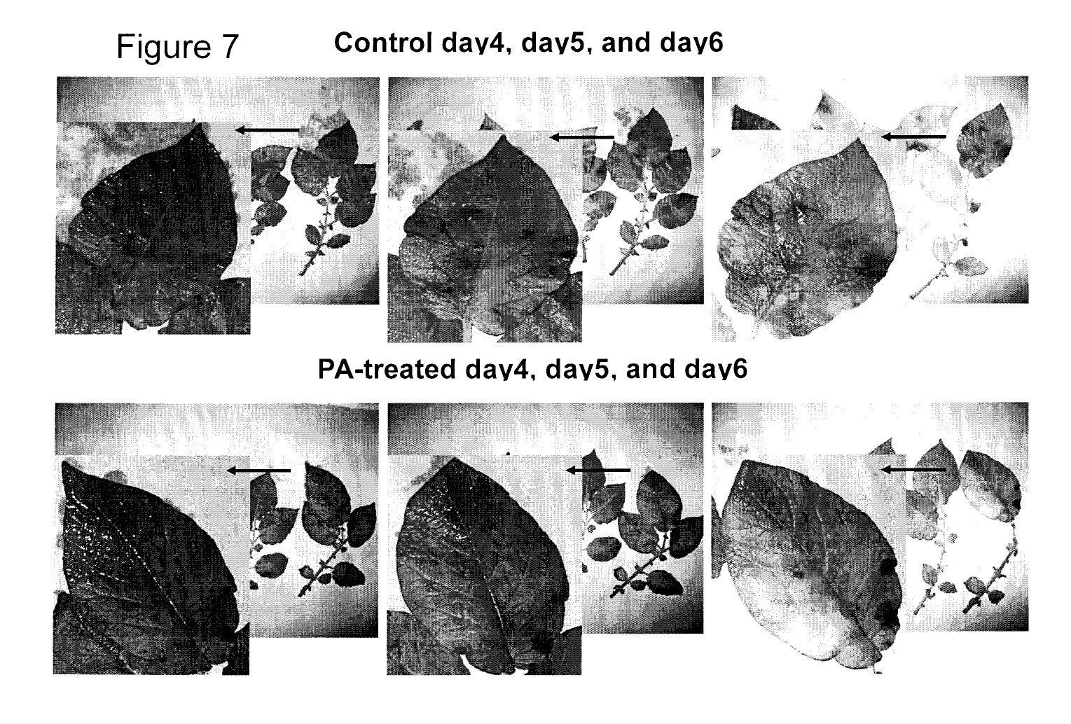

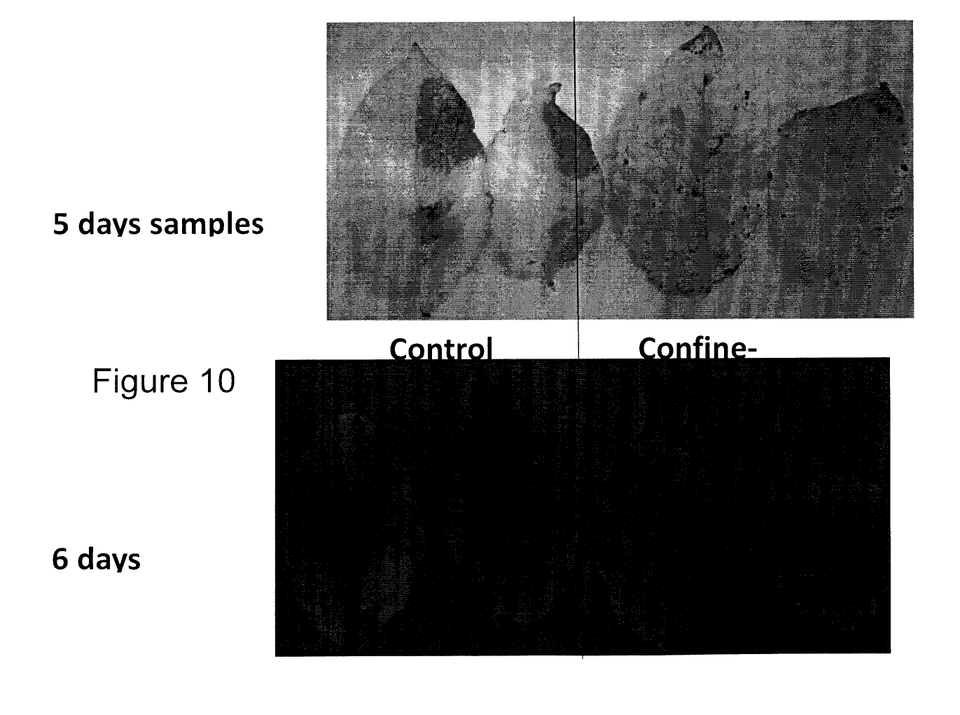

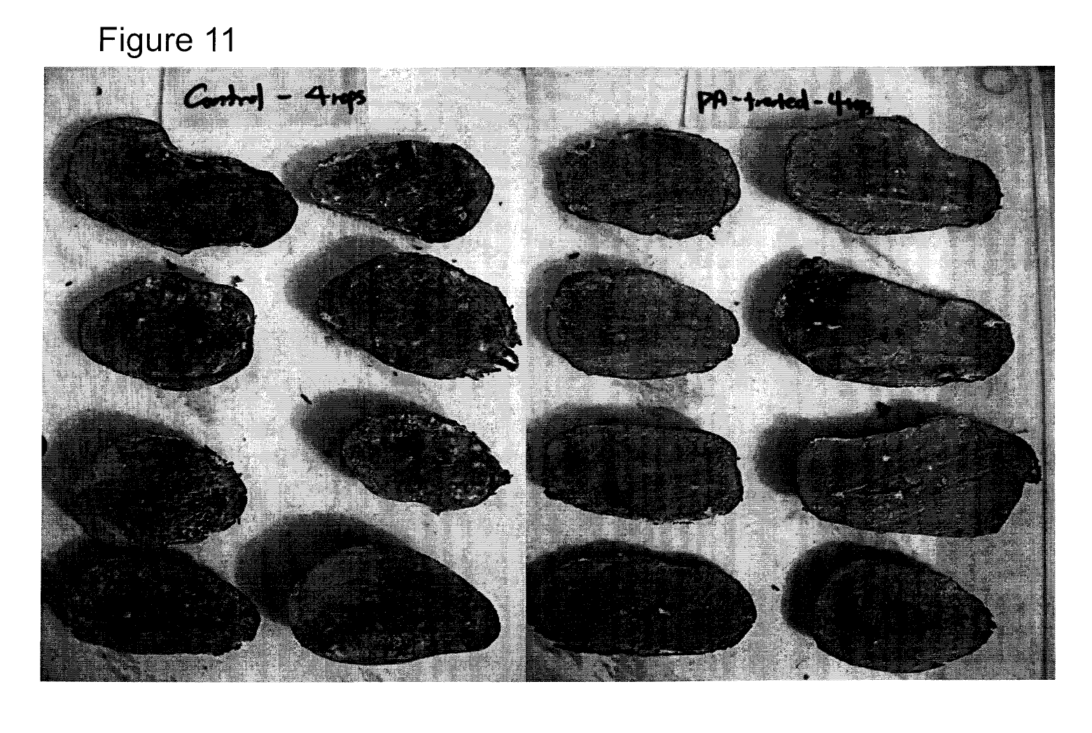

The identified proteins have undergone functional analyses. By analyzing all the proteins identified, almost 9% of reproducible proteins are up- or down-regulated. Therefore, it seems that PA has an indirect mode of action triggering plant proteins, some of which may directly relate to late blight resistance. More than half of the up-regulated proteins (45 out of 72) are involved in plant defense mechanisms; more than half of down-regulated proteins (20 out of 31) play roles in metabolism. The molecular functions of these proteins were categorized as shown in FIG. 2 and FIG. 3. They may be involved in plant-pathogen interaction on plant cell wall. Table 2 lists the 45 up-regulated proteins in the defense category. These proteins were further analysed in PA-treated and non-treated samples before pathogen infection (Table 3) and after pathogen infection (Table 4). Further, one group of proteins, mostly protease inhibitors in PA-treated samples, showed strong response to the pathogen (FIG. 4). Other inhibitors were also identified as shown in Table 5. Some of them are down-regulated in response to pathogen. When analysing the functions of these proteins, it was found that PA-responsive proteins in defense category play roles in defense mechanisms involved in hypersensitive response (HR) and salicylic acid (SA) signaling (Table 6). These processes triggered programmed cell death in leaf tissues, one way to protect plants from pathogen invasion. In order to test this assumption, a 3,3′-Diaminobenzidine (DAB) staining experiment was carried out. This experiment measures the accumulation of hydrogen peroxide which is localized at the infection site by DAB reacting with ROS, leading to the brownish precipitation. The leaf samples treated with PA showed positive DAB staining as shown in FIGS. 5-10. It was also confirmed that tubers harvested from previous year still possessed higher resistance to late blight infection after being stored for 6 months (FIG. 11).

### 4. Protein Validation Experiments

Since the identified proteins hold significant promises to uncover the mode of action of PA on late blight suppression in potatoes, further validation of these proteins in replicated samples is being performed, e.g., leaves from second and third seasons, leaves from growth chamber experiments and tubers with various treatments. Two major methods are implemented: 1) multiple reaction-monitoring, and 2) real-time quantitative RT-PCR. Multiple reaction monitoring (MRM) for targeted quantitative proteomics is a mass spectrometry (MS) based technology. It is used to verify and re-quantify the promising proteins. With the enhanced specificity and sensitivity by MRM, the proteins originally identified are verified. Since some low-abundant proteins, such as those in the HR and SA pathways, cannot all be identified by proteomic profiling, the sensitive real-time quantitative RT-PCR method is used to quantify these proteins. The data from the real-time PCR is combined with the proteomic results to provide the complete view about how the entire PA responses work in plant leaves.

### Materials and Methods

### Field Experiments

Two French fry processing varieties ‘Russet Burbank’ and ‘Shepody’ were grown at the Cavendish Farms' research field located in New Annan, PEI, Canada, in 2007, 2008 and 2009. A two year crop rotation was practiced, potatoes followed by barley under-seeded with clover. The field experimental design was a split-plot with the fungicide treatment as the whole plot factor and cultivar as the subplot factor. Each treatment was replicated four times. The plots were comprised of plot islands each having two 20-foot (6 meters) rows of each of ‘Shepody’ and ‘Russet Burbank’. The two outside rows were guard rows and the two centre rows were used for assessments of the blight in foliage and as harvest rows for yield data and assessment of tuber rot.

The fungicide Bravo® (chlorothalonil) was obtained from Syngenta Crop Protection Canada Inc. (Guelph, Ontario). The commercial product of PA used in the experiments was Confine™, provided by The Agronomy Company of Canada (Thorndale, ON). This PA formulation is a mixture of mono- and di-potassium salts. The four treatments were: 1) plots sprayed with water as control; 2) PA (Confine™) applied alone at the rate of 5.8 L product/250 L water/ha; 3) chlorothalonil (Bravo®) applied alone at the rate of 2 L product/250 L/ha, and 4) PA+chlorothalonil, both at the same rates as individual applications. Fungicide applications were made with a tractor mounted commercial sprayer. Fungicide application took place once a week. In 2007, 10 fungicide applications were made; 8 PA and PA+chlorothalonil applications were made, with water and chlorothalonil applied during weeks 2 and 3. In 2008 and 2009, a total of 11 fungicide applications were made. PA and PA+chlorothalonil applications were made every second spray alternating with water and chlorothalonil, respectively, resulting in 5 applications of each PA and PA+chlorothalonil in 2008 and 6 in 2009.

In 2007, the treatments took place from July 3 to September 10; in 2008, the treatments took place between July 11 and September 16; in 2009, the treatments took place from July 2 to September 11. No late blight was observed in the 2007 field; whereas late blight was found in the fields in both 2008 (started in early August) and 2009 (started in late July). Late blight severity was scored in the field in 2008 from August 20 to September 25 and in 2009 from July 28 to September 3 based on the percentage of defoliation. The score for percent defoliation was based on the total number of plants per plot, the number of infected plants and the severity of the infections (James 1971a).

Pathogen was sprayed on detached leaves days after the PA application (5.8 L product/250 L water/ha). The pathogenicity and proteins were detected 4-7 days after the infection day. The same experiments were repeated in growth chambers when potato plants were grown for 4-6 weeks after emergence. The spraying of the potato leaves PA took place using the same concentration. The rate of application of Confine to each plant grown in a growth chamber was at the rate of 0.2 mL Confine/10 mL water/one plant, to cover the whole plant.

Inoculum of Phytophthora infestans

A local PEI isolate of P. infestans (A2 mating type; US-8 genotype) was used in all studies. This is the same strain that is colonized in the field on the Island. The strain was maintained on excised potato leaves (cv. ‘Green Mountain’) in a humid chamber at 15° C. and transferred weekly to maintain isolate virulence. Inoculum was prepared by swirling infected leaves inoculated 7 days previously in 250 mL of distilled water to dislodge sporangia. The resulting suspension was examined microscopically to determine sporangial concentration (with the aid of a hemacytometer). The inoculum was then diluted to 10-20,000 sporangia/mL and filtered through cheesecloth prior to inoculation.

Inoculation of Detached Leaves with Phytophthora infestans

Asymptomatic leaves from each treatment were detached in early August of each year (Aug. 10, 2007; Aug. 19, 2008; Aug. 4, 2009). Four stems from four plants in each treatment were randomly taken. New leaves from the top were discarded; the first fully expanded leaf from the top (named as P) was taken and frozen in liquid nitrogen for protein analysis. The top second (I-1) and the third (I-2) fully expanded leaves were selected and individually infected. Each leaf was placed in a clear plastic bag, then taken to a nearby shed to prevent infection of the field. Each leaf was inoculated by spraying the adaxial surface with 1 mL of the sporangial suspension (prepared as above). Immediately after spraying, each leaf was sealed in the plastic bag with wet paper towel and then stored in styrofoam boxes with ice packs until being transferred to a growth chamber. Eight leaves per treatment/replicate were collected, for a total of 256 leaf samples in each year. As well, 16 leaves (8 randomly picked from ‘Russet Burbank’ and 8 randomly picked from ‘Shepody’ of water treated plots) were sprayed with water as a negative control for an in vitro infection experiment. The inoculated leaves were incubated in a growth chamber for 7 days at 15° C. with 12 hr photoperiod. The inoculated leaves were evaluated daily for disease severity based on estimating percentage of diseased leaf area (James 1971b). Since no significant disease symptoms occurred prior to day 4, the data used for analysis included only day 4 to day 7 in each year.

### Plant Materials

Potato (Solanum tuberosum L.) var ‘Russet Burbank’ seeds were planted with 30 cm plant spacing and 40 cm in-row spacing. The research plot was located at Cavendish Farms Research Field, Summerside, Prince Edward Island, Canada. The plot layout was a completely randomized block design with four replicates. After three months growth in the field, four plants from each replicate were selected and one fully expanded leaf palm from each plant was collected and frozen in liquid nitrogen immediately. In this study, three biological replicates including total 12 leaves (4 leaf palms per replicate) were used.

Isolation of Cell Wall Proteins from Potato Leaf Tissues

The steps of the extraction method for putative cell wall proteins were based on Feiz et al. (2006) with modifications (FIG. 1). Four grams of potato leaves were ground on ice in MOPS/KOH grinding buffer [50 mM MOPS/KOH, pH 7.5, 5 mM EDTA, 1 mM PMSF, 330 mM Sucrose, 5 mM DTT, 0.6% polyvinyl plypyrrolidone (PVPP), protease inhibitor cocktail (Sigma)]. The homogenate was centrifuged at 1,000 g for 10 min at 4° C. The supernatant was used to extract cytosolic proteins as described in the next section. The pellet was incubated in 10 mL acetate grinding buffer (5 mM acetate buffer, pH 4.6, 0.4 M sucrose, 2 mM PMSF, 0.6% PVPP, 0.2% (v/v) protease inhibitor cocktail) on ice for 30 min with shaking and centrifuged at 1,500 g for 10 min at 4° C. The pellet was resuspended in 10 ml purification buffers (5 mM acetate buffer, pH 4.6, 0.2% (v/v) protease inhibitor cocktail) containing 0.6 M sucrose. After centrifugation at 1,500 g for 10 min at 4° C., it was resuspended in the same buffer 10 ml but containing 1 M sucrose (explain why). The pellet was then vortexed with the extraction buffer (5 mM acetate buffer, pH 4.6, 0.1% (v/v) protease inhibitor cocktail, 0.2 M CaCl₂) for 10 min at 4° C. and then was centrifuged at 10,000 g for 20 min at 4° C. Trichloroacetic acid (TCA) was added to the supernatant to a final concentration of 10%. The mixture was inverted, vortexed, and stored at −20° C. for 30 min. The pellet was resuspended in urea buffer (8 M urea, 100 mM Tris-HCl, pH 8.5) as the wall protein fraction and stored at −80° C. until further use.

Isolation of Cytosolic Proteins from Potato Leaf Tissues

For cytosolic proteins, the supernatant obtained from the centrifugation at 1,500 g was ultracentrifuged at 100,000 g for 1 hour (FIG. 1). TCA was added in the supernatant to 10% final concentration and mixed well by vortexing and then kept for 30 min at −20° C. It was centrifuged at 16,000 g for 5 min at 4° C. and the pellet was successively washed with 80% methanol, 0.1 M ammonium acetate and finally 80% acetone. The pellet was air dried and resuspended with 400 μl SDS buffer (30% sucrose, 2% SDS, 0.1 M Tris-HCl, pH 8.0, 5% β-mercaptoethanol) and then 400 μl phenol was added. The mixture was then mixed well by inversion and incubated for 5 min at room temperature. It was centrifuged at room temperature at 8,000 g for 10 min and 400 μl upper phenol phase was transferred to a new tube. The tube was filled with 100% methanol containing 0.1 M ammonium acetate. It was stored for 30 min at −20° C. and was centrifuged at 16,000 g for 5 min at 4° C. and then the pellet was washed with 100% methanol and 80% acetone, successively. The pellet was resuspended in the urea buffer as the cytosolic protein fraction and stored at −80° C. until further use. All chemicals were purchased from Sigma-Aldrich (St. Louis, Mo.).

### Digestion of Proteins for LC-MS/MS

The cell wall and cytosolic proteins were quantified by Bradford assay (Bradford, 1976). One hundred micrograms of cell wall proteins and five hundred micrograms of cytosolic proteins, respectively, were reduced with 5 mM DTT for 30 min at 60° C., alkylated with 15 mM iodoacetamide for 30 min at room temperature in the dark and then digested with trypsin (1:50 w/w trypsin/protein ratio; Promega, Madison, Wis.) at 37° C. overnight. Digested peptides were desalted using C18 Sep-Pak cartridge with 0.5 ml 50% acetonitrile and then 0.5 ml 100% acetonitrile.

### Two-Dimensional LC-MS/MS

LC-MS/MS was performed using an Agilent 11000 LC coupled to 4000 Q-TRAP mass spectrometry (Applied Biosystems Inc, USA). Peptides were fractionated by strong cation exchange (SCX) using the LC equipped with a 100×2.1 mm² polysulfoetyl A column (PolyLC, Columbia, Md., USA) into 30 fractions at a flow rate of 0.2 mL/min using a linear gradient from 10 to 600 mM ammonium formate over 70 min. Each fraction was dried by speed vacuum and dried fractions were redissolved by adding 25 μl of mobile phase A (0.1% formic acid in water). Peptides in each fraction were separated by reversed-phase liquid chromatography on an Agilent 100 CapLC equipped with a 150×0.1 mm monolithic C18 (Phenomenex, CA, USA) coupled to the Q-TRAP mass spectrometry. An electrospray voltage of 5500 was applied to the 30 μm spray tip (New Objectives, MA, USA). A linear gradient from mobile A (5% acetonitrile, 0.2% formic acid) to mobile B (80% acetonitrile, 0.2% formic acid) over 50 min was applied at the flow rate of 2 μl/min. MS/MS spectra from the mass spectrometry were collected by IDA acquisition.

### Protein Identification

The MS/MS spectra were searched against the potato gene index database (http://compbio.dfci.harvard.edu/cgi-bin/tgi/gimain.pl?gudb=potato; release 12.0) using the Mascot v. 2.1.0 search engine (http://matrixscience.com, Matrix Science, London, UK). The search parameters included fixed cystein carbamido-methylation, variable methionine oxidation, a maximum of one missed tryptic cleavage site, peptide tolerance of ±1.2 Da, MS/MS tolerance of ±0.8 Da. A significance threshold of p<0.05 and the ions score of 30 or greater in Mascot for an MS/MS match were used. False discovery rate for the estimate of false positives among the matched peptides was performed in the decoy (reversed) database (Wright et al., 2009).

### Bioinformatic Analysis

Several servers and databases were used in this study. SignalP server allows predicting the presence and location of cleavage sites for a signal peptide (http://www.cbs.dtu.dk/services/SignalP/) (Bendtsen et al., 2004; Emanuelsson et al., 2007). Cytoscape (http://cytoscape.org) is open software supporting visualization and integration of genes' network (Cline et al., 2007). BiNGO as a Java-based tool is implemented as a plugin for Cytoscape to determine gene ontology for an interesting set of genes (Maere et al., 2005). To analyze gene ontology (GO) for interesting genes using Cytoscape, we used GO annotation files (http://www.geneontology.org/GO.downloads.annotations.shtml) against Arabidopsis available at NCBI. TC (tentative consensus) numbers in potato gene index database were associated with Arabodopsis TAIR identifiers by selecting the top match from the BLAST (blastn) output of the nucleotide potato sequences versus a TAIR nucleotide BLAST database. The TAIR identifiers were used to extract gene descriptions from the KEGG database.

### Example 2

After the proteomic profiling, several approaches were taken to examine the functions of Confine in relation to its late blight suppression in potatoes. In doing so, a series of proteins were validated using either multiple reaction monitoring (MRM) or real-time quantitative reverse transcription PCR (real-time qRT-PCR). These validations further confirmed that these proteins indeed are regulated by the application of Confine in potato leaves. Some of these proteins are involved in salicylic acid (SA) signaling pathway that triggered the ROS production and subsequent hypersensitive response (HR), which are responsible for the late blight suppression in potato leaves. In addition, some other proteins have shown antifungal functions and functions related to plant defense mechanisms, pathways related to general metabolism and energy production, and those involved in starch and sugar metabolism.

In order to confirm that HR response was induced by Confine, several experiments were established confirming directly or indirectly that programmed cell death, a signature HR response in plants, is indeed triggered by Confine that had resulted in the control of the pathogen spread in plant leaf tissues.

To confirm the direct inhibition of the late blight pathogen, infection experiments were carried out in whole plants as well as in tubers. Confine was able to inhibit the growth and development of the pathogen organism in both leaf and tuber tissues. Such inhibition resulted in slower spread of the disease symptoms, causing less damage to the plant tissues.

### 1. Protein Validation

The 103 proteins originally identified in Example 1 were further evaluated, because these proteins were identified using a bioinformatics Mascot program that is semi-quantitative. Two individual methods were used for this validation, multiple reaction monitoring (MRM) and real-time quantitative reverse transcription PCR (Real-time qRT-PCR). Both methods are the best in art for quantitative analysis of protein and gene expression.

In MRM, 15 proteins were tested. Ten of them are from Table 1, with 8 of the 10 showed increased abundance and 2 of the 10 showed decreased abundance. The other 5 proteins listed in Table 7 were newly chosen because they belong to the same families of proteins, e.g. peroxidases, beta-1,3-glucanases, cysteine protease inhibitors, serine proteases (subtilisin), or aspartic protease, and it is important to know if their protein abundance is regulated by Confine.

In real-time qRT-PCR validation, 15 proteins were tested using the primer pairs designed (Table 10). Seven (7) of the proteins (shaded in Table 10) were from Table 1. The other 8 are newly selected, they are mostly protein families related to these proteins.

### 1.1 MRM

MRM technology is the best method to validate proteins after proteomic profiling with high accuracy. Ten promising proteins from Table 1 were chosen. All of them have been confirmed to be comparable to data from Example 1, meaning that if protein abundance was increased in Example 1, it had shown the increase in MRM validation, and vice versa. 5 new proteins were also tested (Table 7) based on their functional relationship to the other proteins identified.

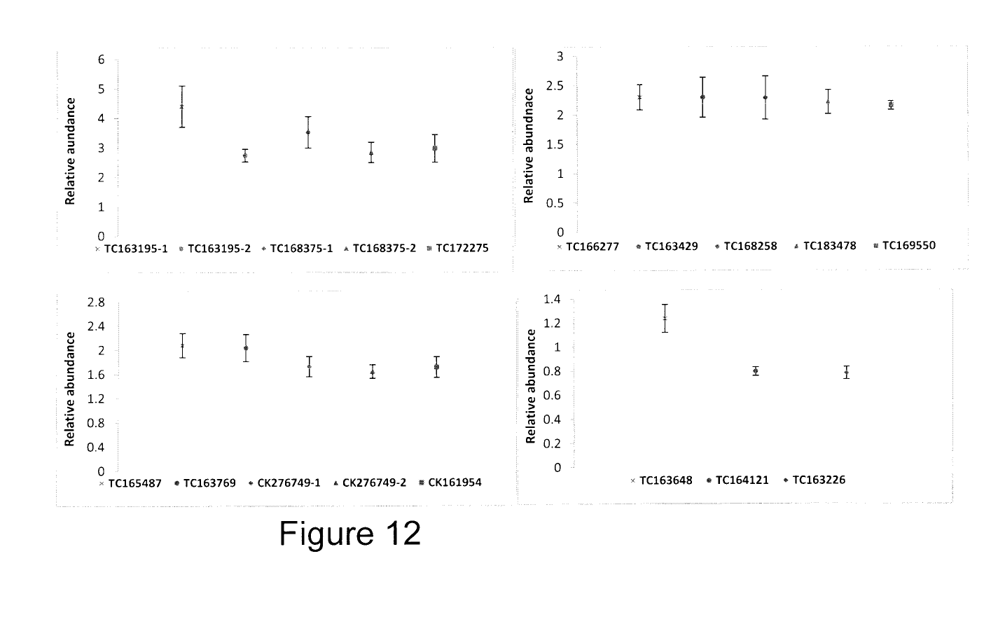

The results from MRM validation is shown in Table 8 and FIG. 12. The 8 proteins originally categorized as up-regulated are confirmed to be up-regulated; the 2 proteins originally categorized as down-regulated are confirmed to be down-regulated. The 5 newly selected proteins are all up-regulated.

In proteomic profiling, several proteins, such as peroxidases (TC 172434, TC169870, TC164504), beta-1,3-glucanases (TC173865, TC163195), and cysteine protease inhibitors (TC181645, TC166886, TC166762), were induced by Confine application. Therefore, other similar proteins were tested for regulation by Confine. Peroxidise (TC166277), beta-1,3-glucanase (TC183478), and cysteine protease inhibitor (TC169550) were chosen and the results confirmed that similar proteins are regulated.

The abundance pattern [up-regulation by Confine application, down-regulation by P. infestans 4 dpi (days post infection)] of aspartic protease inhibitor showed the same as cysteine protease inhibitor in quantitative proteomics. So, other aspartic protease inhibitors were tested for regulation by Confine. Aspartic protease inhibitor CK163648 confirmed this regulation.

Cysteine proteases were up-regulated by Confine application in the proteomic profiling. Subtilisin, a serine protease which is another type of protease is known as having similar function to cysteine protease depending on plants. Therefore, a subtilisin (CK276749) was chosen to confirm the abundance of serine protease, another type of protease.

For late blight suppression, peroxidase may be involved in establishing cell wall reinforcement and the induction of hypersensitive response (HR). Beta-1,3-glucanase as secretory protein with antifungal activity is believed to directly degrade pathogen cell walls. Caspases including cysteine protease and aspartic protease are involved in HR. Therefore, cysteine protease inhibitor and aspartic protease inhibitor may play roles in regulating HR and inhibiting proteases secreted from P. infestans. Subtilisin-like protease may be an alternative to proteases involved in HR in plants. Subtilin-like PR protein was accumulated in tomatoes for putative defense response upon P. infestans (Tian et al., 2007). Therefore, all these validated proteins are likely to have defense functions against P. infestans.

The summarized functions of these 15 validated proteins are listed in Table 9. As shown, the proteins induced by Confine are acted as antifungal factors for the direct mode of action and/or stimulated the SA signalling pathway. This pathway triggers the production of ROS, resulting in the activation of hypersensitive response (HR). HR is a well-known response plants for defense against pathogen attack.

1.2 qRT-PCR

The qRT-PCR technology is a commonly accepted method to validate proteins from proteomic profiling and genes from microarray. It is based on the use of a primer pair and the cDNA template that is reversely transcribed from the mRNA of a biological sample. It is quantitative because the gene expression is measured real time based on the amount of the template. Total 15 genes were evaluated (Table 10), and 7 of them matched with the 7 proteins in the original 103 proteins from Example 1 based on their DNA and peptide sequences. The DNA primers used for qRT-PCR could amplify a family of proteins if they share the same DNA sequences (gene families). Therefore, design primers that are allele specific were tried, which resulted in 8 more proteins shown as in Table 10. The results from the qRT-PCR provide a trend on how the families of the proteins respond to Confine treatment.

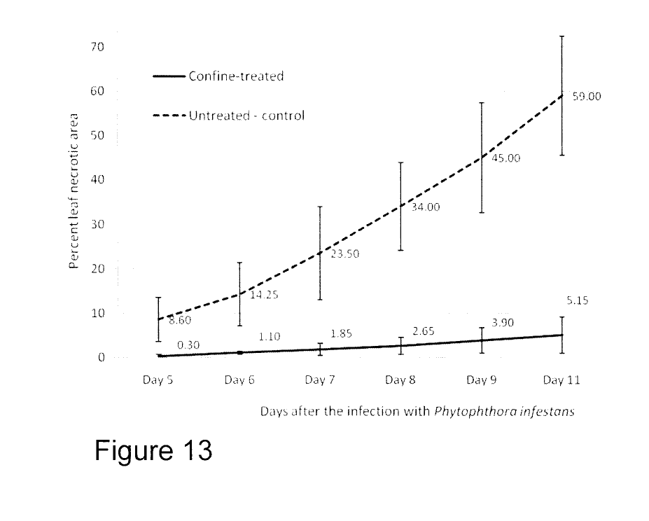

Plants that were analysed by qRT-PCR were infected 0 days after Confine treatment and the infection rate was recorded. Four days after inoculation the first sign of infection (necrotic spots) were visible on both untreated and Confine-treated plants. Systematic recording of the progression of infection (percentage of leaf covered by necrotic spots and the occurrence of sporangiophores harbouring sporangia) was performed from day 5 post-inoculation to day 11 (FIG. 13). A delay in the progression of the disease was observed in Confine-treated plants; the suppression of late blight development on potato plants after only one treatment with 1% Confine clearly indicate the efficacy of this antifungal agent. Five weeks after the infection with P. infestans, at the end of the experiment, the percentage of foliar necrosis of untreated plants was >95% while that of Confine-treated plants was <20%.

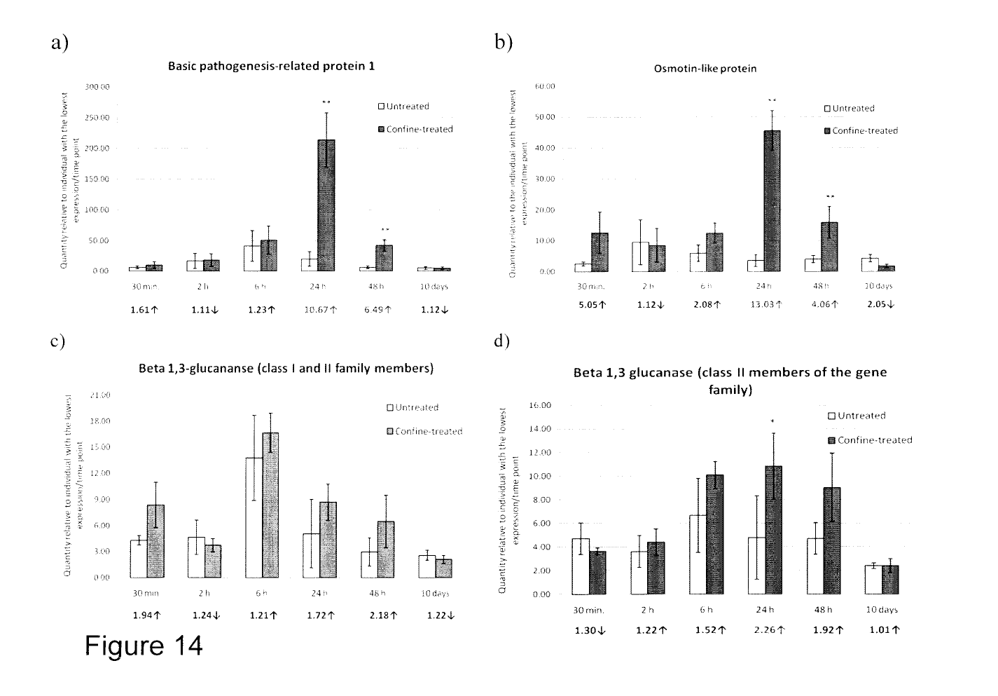

The qRT-PCR results of each individual gene are summarized in FIGS. 14, 15 and 16. Graphics represented the 6 time points for each of the genes/gene families analyzed from the experiment employing potato plants treated with 1% Confine. Significance level shown are * p<0.1; ** p<0.05. Table 11 summarized the level of the 15 genes with their expression levels in comparison with the proteomic data. The bolded gene names are the genes that are equivalent to the protein sequence obtained by the proteomic profiling. Remarkably, proteomics data and qPCR data are highly convergent; conflicts are only minor. This is better than expected if we take into account that different samples have been analyzed and the number of treatments with Confine was different. Also, one experiment was performed using samples from field potatoes while the other one was conducted using samples from potatoes grown in more controlled conditions, e.g., in growth chamber.

Below are the analyses of qRT-PCR data supporting the proteins found by proteomics.

### 1) Validation of a Subset of the Proteins Found to be Up-Regulated in the Proteomics Analyses.

Proteomics analyses suggest that a number of proteins are up-regulated by Confine applications. Based on their function/roles in various biological systems, these proteins can be classified in 5 categories: i) proteins primarily involved in defense mechanisms; ii) proteins functioning in metabolic pathways and energy production; iii) proteins related to synthesis/protein turnover; iv) proteins that are involved in signal transduction and v) proteins whose function remain to be determined (unknown function). The first category, related to plant defense mechanisms comprises most of the genes that are up-regulated (>40 proteins; see Table 1A). Most of the proteins identified by proteomics as up-regulated by Confine and involved in defense mechanisms correspond to rather large gene families (e.g. pathogenesis-related proteins, osmotins, glucanases, chitinases, peroxidases). For qRT-PCR, a number of genes representing the defense mechanisms were selected. In addition, the primers used to analyze the expression of these genes by qRT-PCR were designed to amplify as many members of the gene family (i.e., primers were designed in regions conserved among the various members of the same gene family). The aim of qRT-PCR analyses was twofold: i) to validate the expression of several genes that encode the proteins identified in proteomics and, ii) to validate the general trend of up-regulation observed in many genes/proteins involved in the defense response in plants. The qRT-PCR analysis at different intervals (time-points) after plant treatment with Confine of the expression of several members of basic pathogenesis-related protein 1, osmotin, beta 1,3 glucanases (class I and II) and separately of beta 1,3 glucanases from class II confirmed a general trend of gene up-regulation observed in proteomics. The peak of up-regulation is reached 24 h to 48 h after Confine treatment. The same trend of up-regulation was observed for these genes after infection with P. infestans (Table 12).

### 2) Validation of a Subset of the Proteins Found to be Down-Regulated in the Proteomics Analyses.

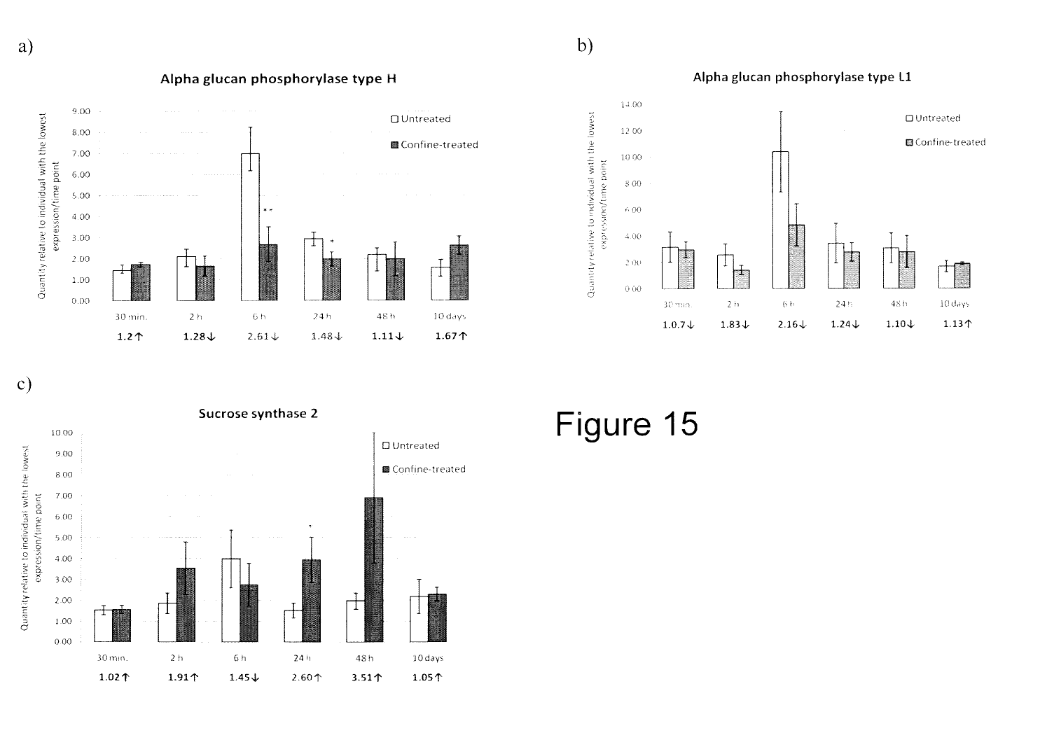

Proteomics data indicate that many of the proteins that are down-regulated belong to pathways related to general metabolism and to energy production (see the Table 1b for the proteins that proteomics indicates to be down-regulated). Analysis of a subset of genes encoding proteins identified by proteomics, e.g. of alpha glucan phosphorylase type H and type L1 confirmed a down-regulation gene expression trend after Confine application (FIGS. 15a and 15b) but a rather unchanged expression after infection with P. infestans (Table 12). In contrast with proteomics data sucrose synthase 2 exhibits an up-regulation gene expression trend after Confine treatment (FIG. 15c) and infection with P. infestans (Table 12). However, one time point out of six indicates that sucrose synthase 2 can be slightly down-regulated in the second half of the light period (the 6 h time point after Confine treatment corresponds to 10 h of the light period; plants were grown on a the dark/light cycle of 16 h of light and 8 h of dark).

3) Genes Analyzed by qPCR Whose Products (Proteins) Perform Cellular Functions Related to Those Identified Using Proteomics.

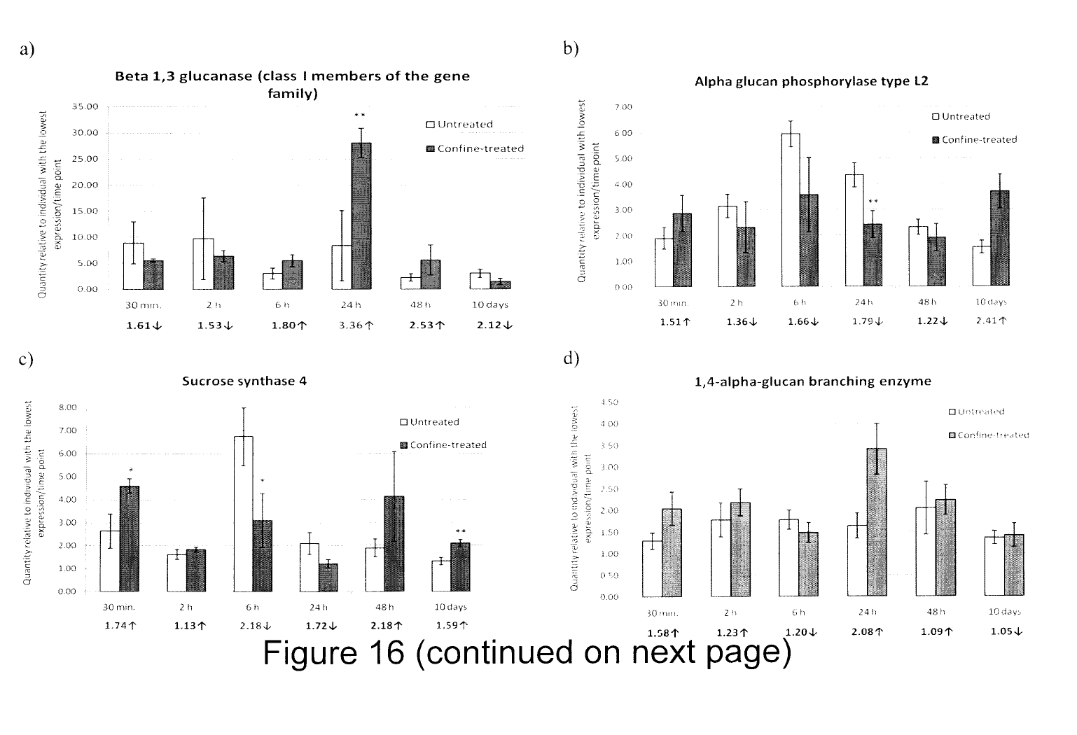

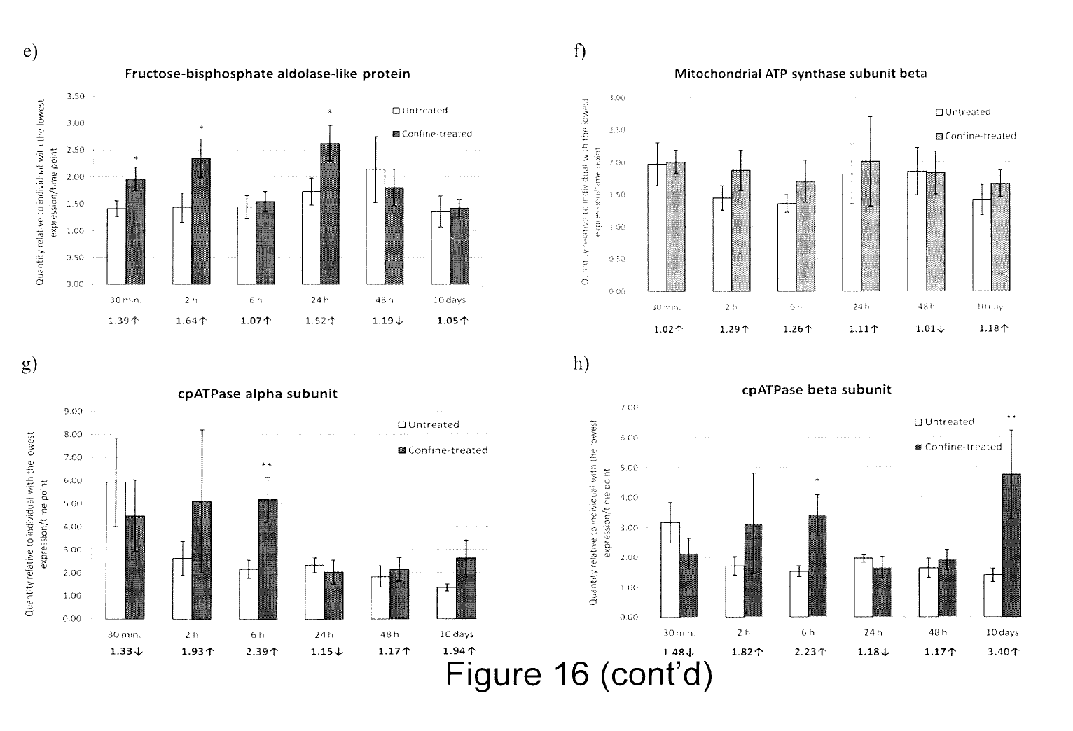

A few genes were analyzed by qRT-PCR to reinforce the trend found by studying the expression of genes encoding the proteins identified by proteomics: class I beta 1,3 glucanase (involved in defense mechanisms), alpha glucan phosphorylase type L2 and sucrose synthase 4 (involved in starch and sugar metabolism) (FIG. 16a, 16b, 16c). Analyses confirmed the up-regulation of the class I beta 1,3 glucanase (FIG. 16a) and the down-regulation of alpha glucan phosphorylases (FIG. 16b). The results from sucrose synthase 4 confirmed that these enzymes tend to be down-regulated in the second half of the light period (FIG. 16c); however the general gene expression trend is that of up-regulation, including the expression in P. infestans infected leaves (Table 12). The analysis of the other genes involved in sugar metabolism (FIGS. 15d and 15e) and energy generation (FIG. 15f, 15g, 15h) indicate a general up-regulation trend in gene expression including the expression in P. infestans infected leaves (Table 12). This finding reinforces proteomics data that show that proteins such as glyceraldehydes 3 phosphate dehydrogenase, triose phosphate isomerase (glycolytic enzymes) and succinyl-CoA ligase (tricarboxylic acid cycle), which function in related pathways, tend to be up-regulated.

### 2. Programmed Cell Death Induced by PA

Based on the proteins PA induced, it was believed that PA enhanced hypersensitive response (HR) related cell death in Confine treated plant leaves. This response is responsible for plant protection against late blight.

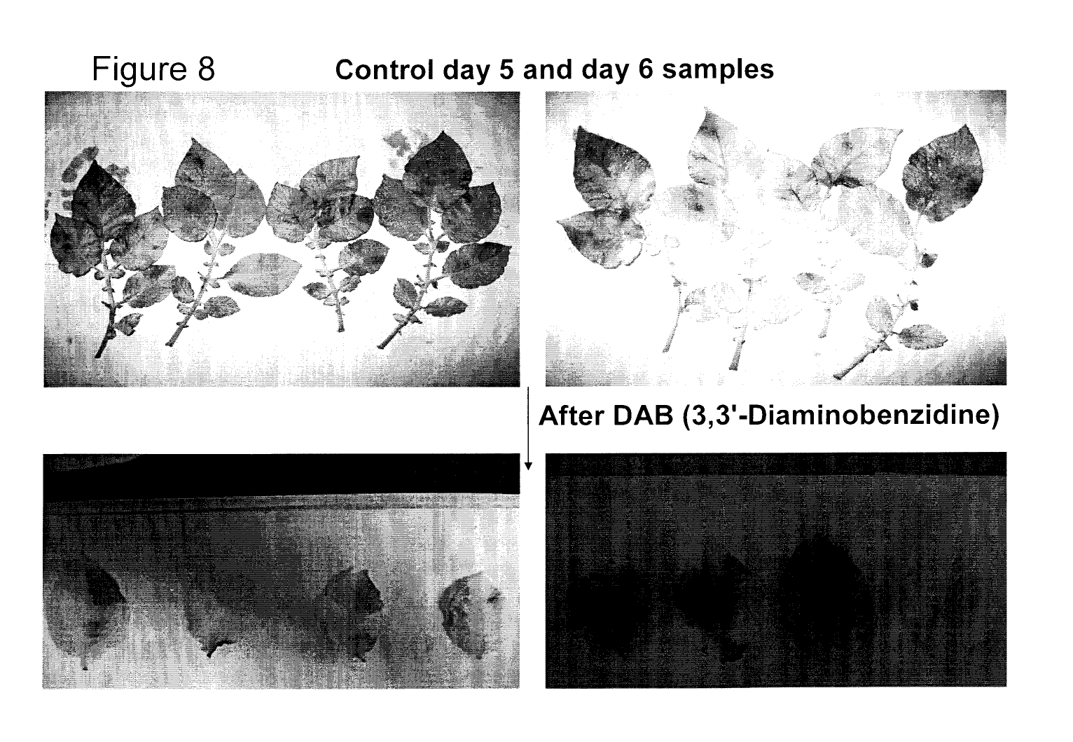

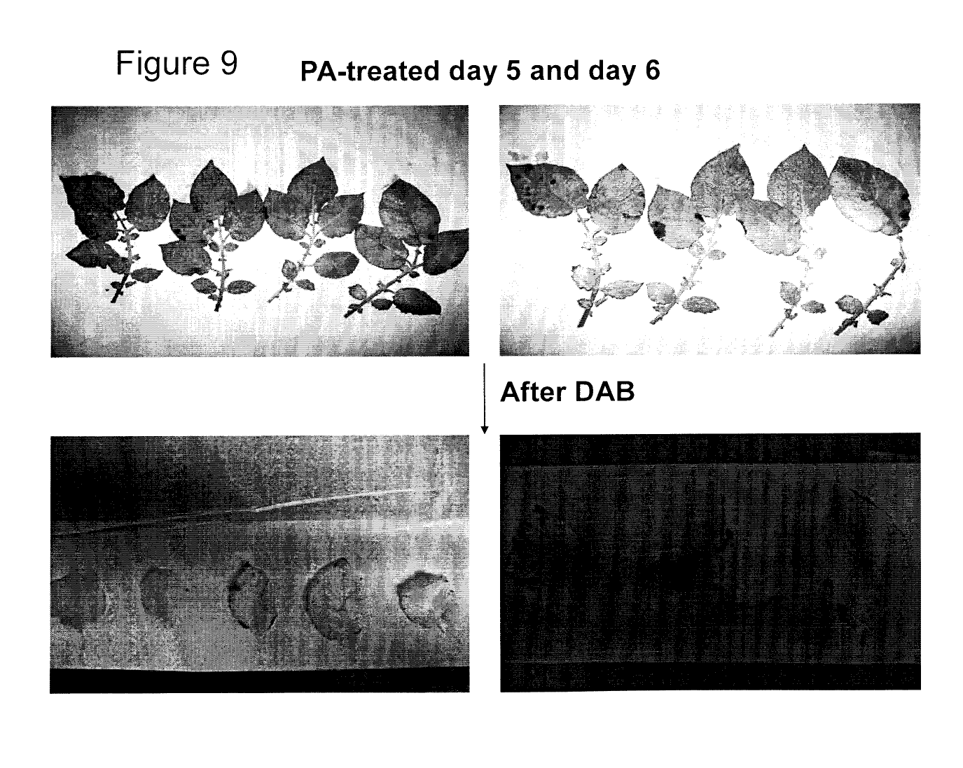

In Example 1, the results of H₂O₂ evaluation were presented in Confine treated leaves days after pathogen infection (FIGS. 8, 9, 10). It was the first evidence of ROS in Confine treated plant leaves.

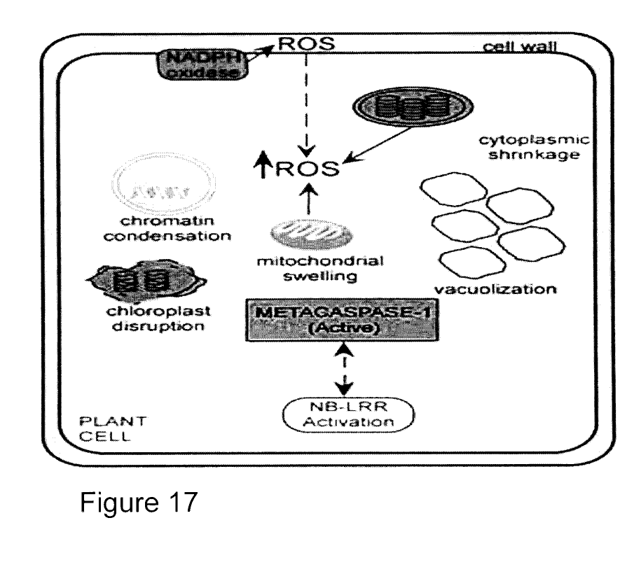

To further develop detailed investigation, two experiments were used to observe HR cell death: 1) microscopy observations using light microscope (LM), stereo electron microscope (SEM), and transmission electron microscope (TEM); 2) callose deposition. Individually established growth chamber grown potatoes that were treated either by water (as control) or Confine were used. FIG. 17 shows the programmed cell death symptoms typically occurring in cells with hypersensitive response (HR).

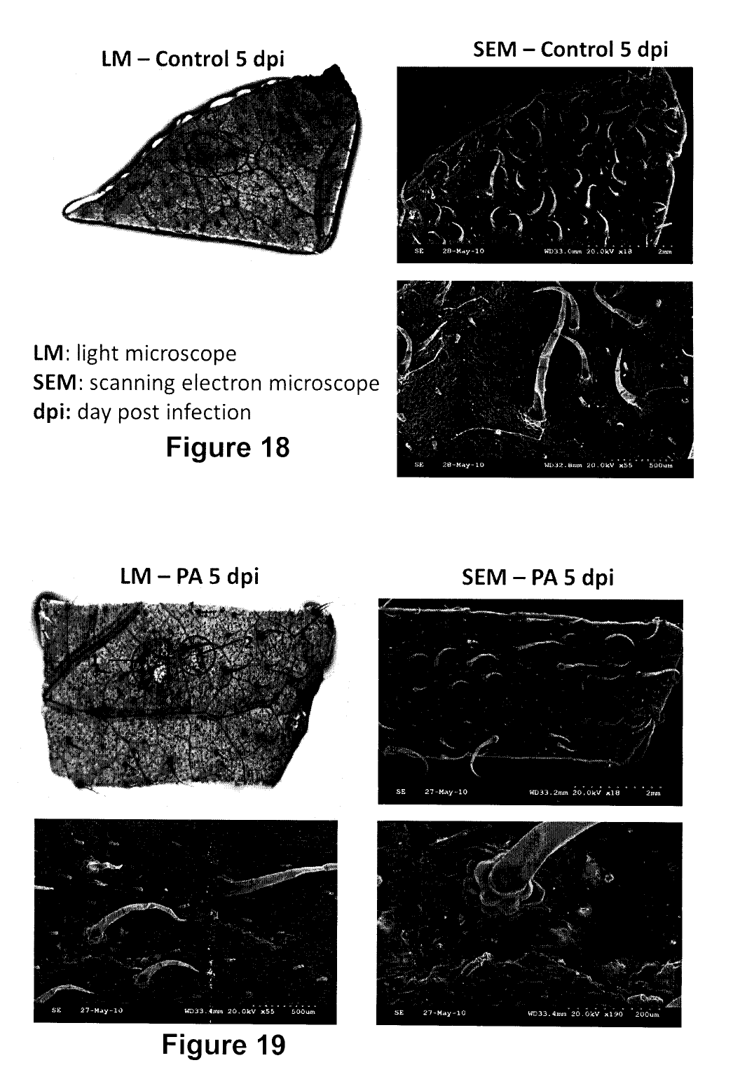

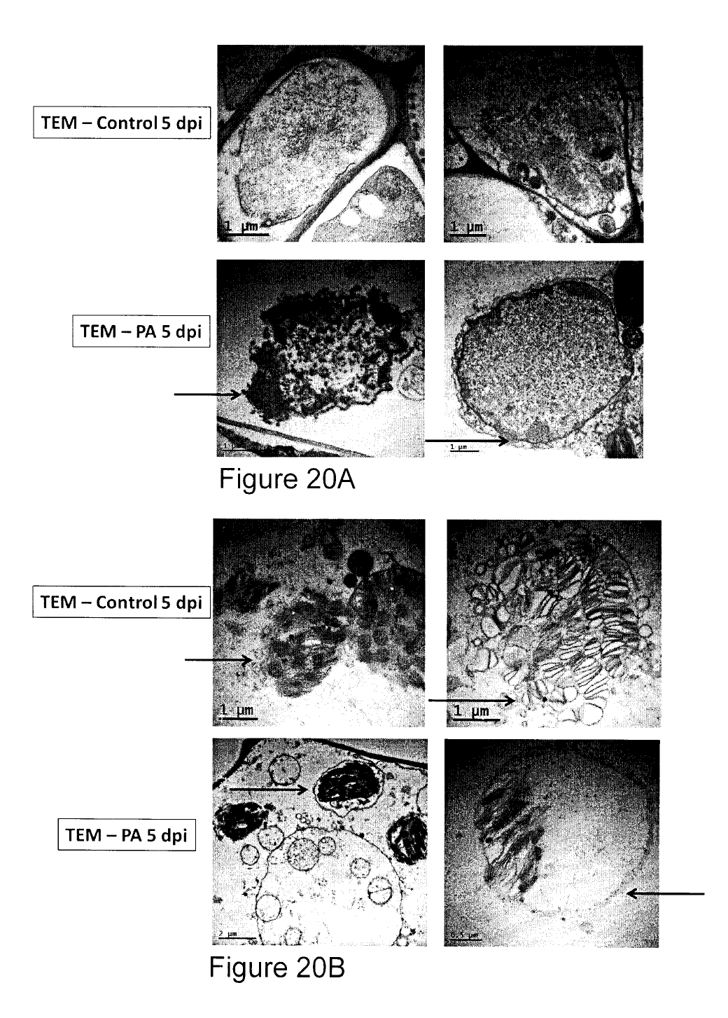

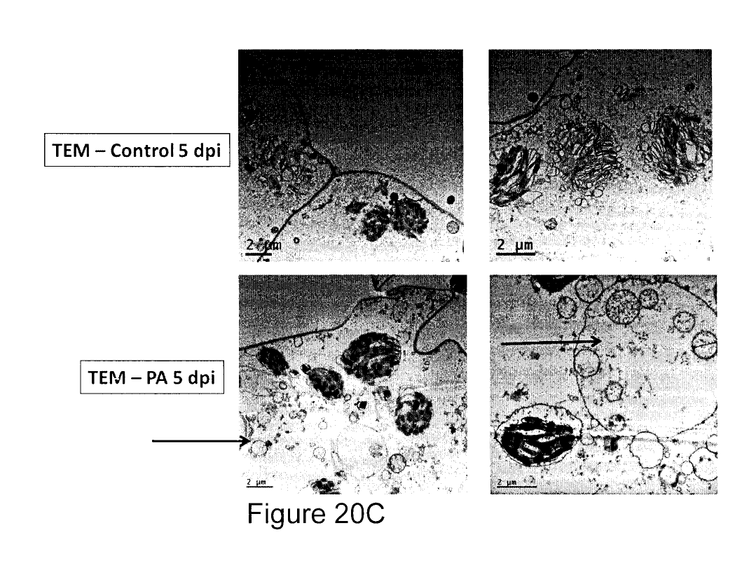

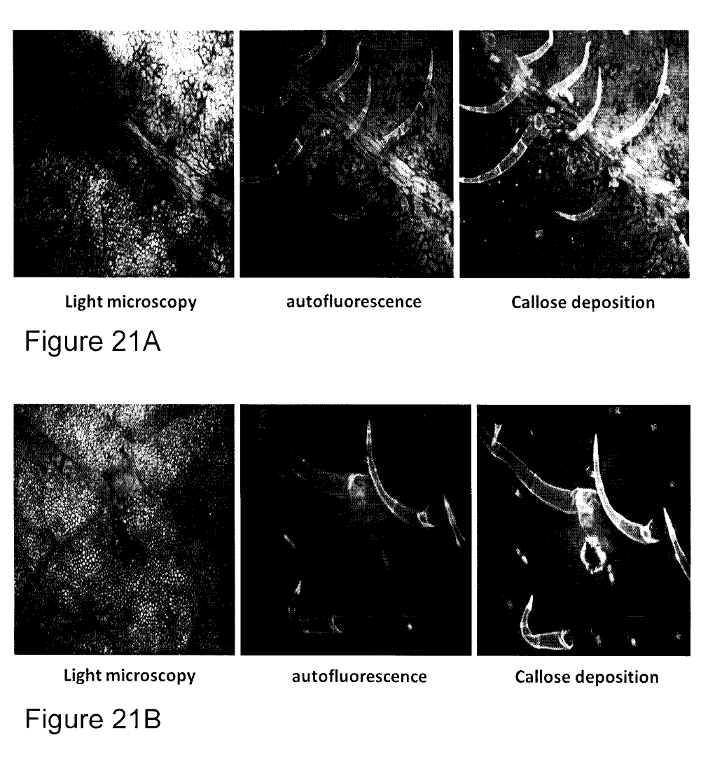

FIGS. 18, 19, and 20 demonstrated the representative results of cell death observed under LM, SEM and TEM. FIGS. 21A and 21B showed the results from callose deposition analysis in Confine treated and control plant leaves. These results together with that included in Example 1 strongly support the hypothesis that Confine activated the HR related cell death after pathogen attack, which resulted in inhibition of the spread of the disease.

### 3. Phenotypic Analysis of Confine's Function on Delay of Late Blight Development on Potato Leaves and Tubers

### 3.1 The Effects of Confine on Late Blight Growth on Potato Slices—Experiment One

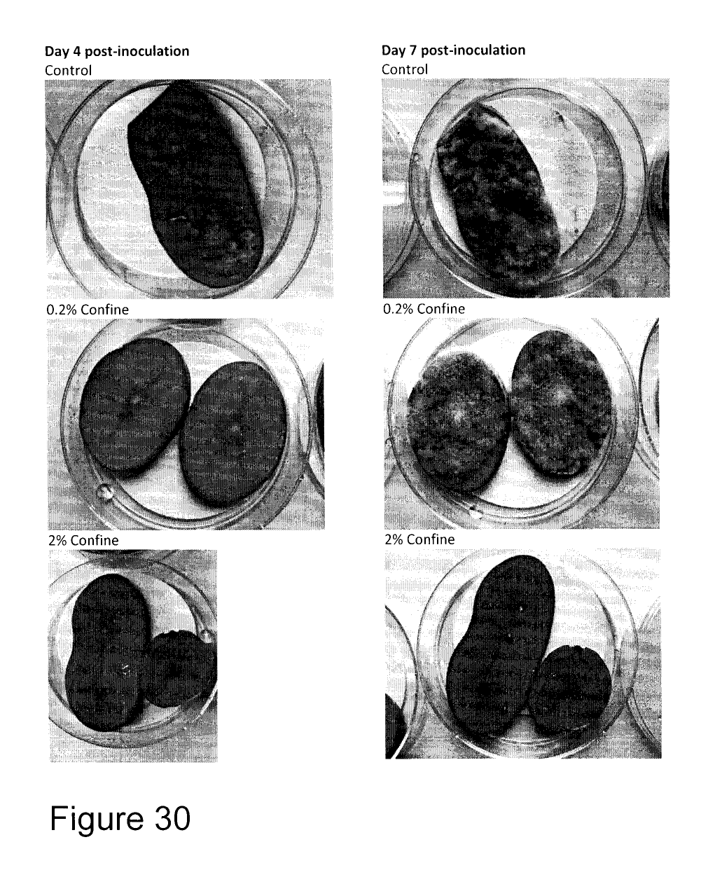

Potato tuber slices (var. Shepody) were submerged for 2-3 seconds in water or in two different concentrations of Confine (0.2% or 2% Confine) prior to inoculation with Phytophthora infestans A2 US8 strain. Pathogen inoculation on potato slices was performed by transferring with a sterile loop sporangiophores with sporangia to the center of the potato slice (FIG. 30). The experiments contained 4 replications in each treatment. The treatment with 0.2% Confine delayed Phytophthora infestans growth; 2% showed almost complete inhibition of Phytophthora infestans growth.

3.2 Carry-Over Effect of Confine on Late Blight Suppression in Tubers from Plants Treated in Fields—Second Experiment

### Experiment Summary

Potato tubers derived from plants that had been treated with confine were more resistant to Phytophthora infestans infection, than control tubers derived from untreated plants. This experiment was repeated two times for three varieties (Russet Burbank, Shepody, Prospect) of potato tubers. In each instance the visual symptoms of infection by P. infestans were delayed by one to two days. These data confirm that Confine, when applied as a foliar spray to the field plants during the growing season, is transported into the potato tubers, where it accumulates to a high enough level to provide some protection against late blight infection.

### Methods

Phytophthora infestans (A2 mating strain US8) were obtained from Rick Peters (AAFC, Charlottetown, PE) and propagated on potato slices by transferring sporangia to new slices every 5 to 7 days. Sporangia were harvested by washing infected slices in sterile water, filtering through a 30 μM nylon mesh to obtain sporangia, washing the sporangia in sterile water and captured them with 10 μM nylon mesh. Zoospores were obtained by incubating sporangia at 10° C. for 3 hours, the concentration was verified by counting on a hemocytometer.

Potato tubers were harvested from Cavendish Farm's research plot (PEI) in the fall of 2010. Throughout the 2010 growing season, some plants received foliar treatments of Confine (Treated) while others remained untreated (Control). The tubers were stored at 4° C. until these experiments were conducted in March and April 2011. At the time of the experiment, tubers were washed in distilled water, immersed in 0.3% sodium hypochloride for 20 minutes, rinsed with sterile water and then cut into six slices of 5-7 mm thickness. One slice from each tuber was saved for chemical analysis the others were washed, placed on moist (400 μl of sterile water) filter papers in Petri dishes and 50 μL of Phytophthora infestans (1,000 zoospore/mL) was spread on the surface with a hockey stick. They were sealed with parafilm and incubated at room temp (18-20° C.) with 14 hour light period for about 5-7 days. Infection was assessed by counting the number of sporangia per slice using a hemocytomer, or by visually assessing the infected area. Statistical ANOVA was applied to the data using SAS.

### Results

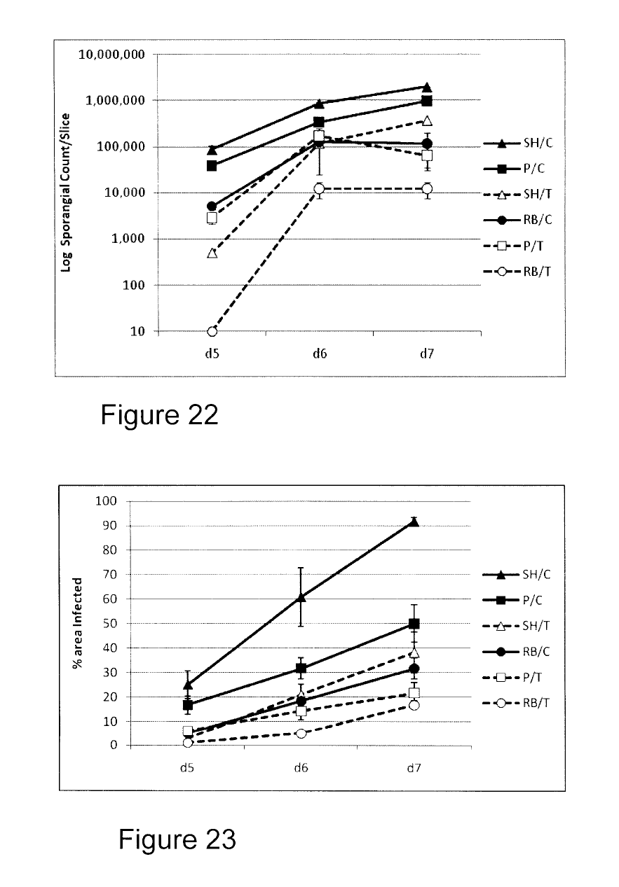

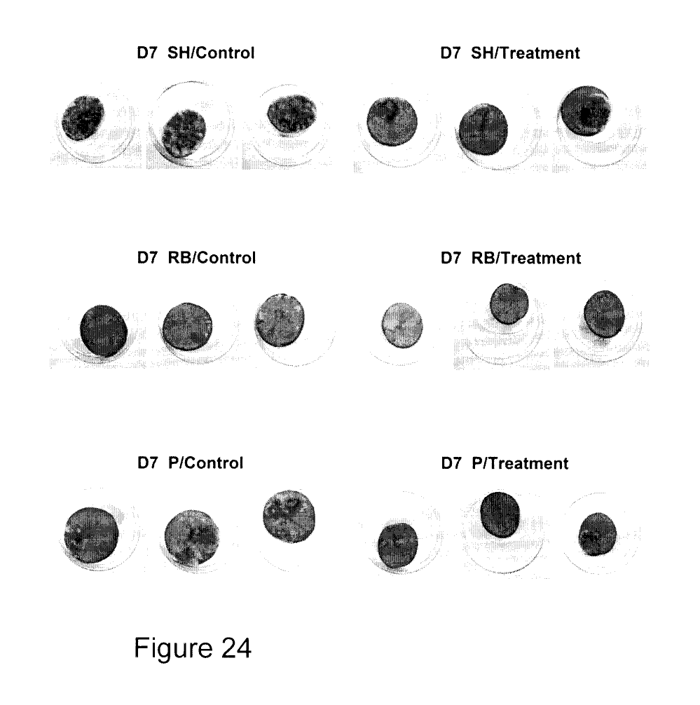

In the first tuber experiment, two tubers were used per cultivar/treatment. Sporangial counts (FIG. 22) and area of infection (FIG. 23) of control tubers reveal that Shepody was the most susceptible to Phytophthora infestans infection, followed by Prospect and then Russet Burbank. For each variety, tubers from Confine treated plants were less susceptible to Phytophthora infestans infection than their untreated controls. These differences were statistically significant for Shepody and Prospect. Photos of these slices at Day 7 confirm these trends also (FIG. 24).

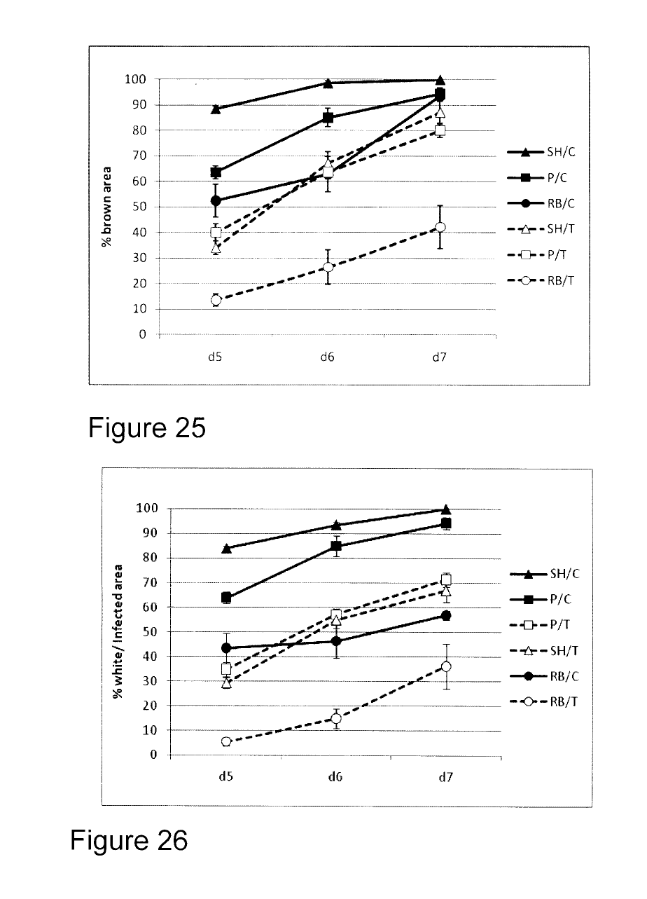

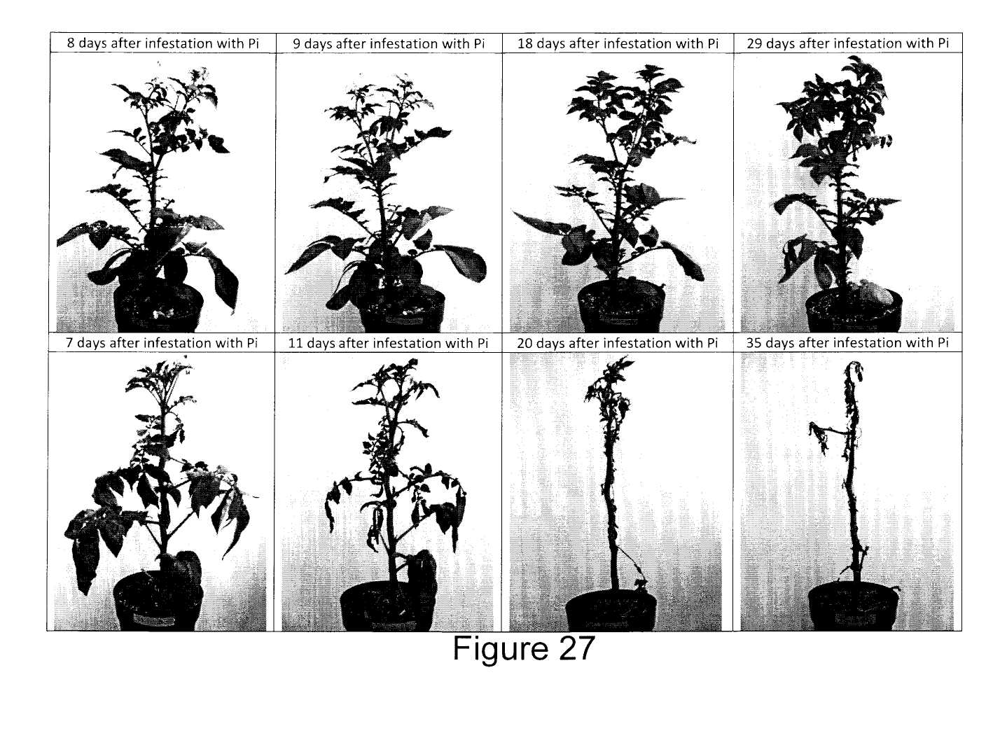

In the second tuber experiment, two tubers were used per cultivar/treatment. Estimates of damage to tuber tissue (percent brown area; FIG. 25) and estimates of P. infestans growth (percent area covered with white; FIG. 26) closely mimic the results of the previous experiment. Cultivar susceptibility decreased from Shepody to Prospect to Russet Burbank; as well, tubers from Confine-treated plants were less susceptible to Phytophthora infestans. This second experiment differed from the first in that the overall level of infection was higher. This may have been caused by subtle differences in culturing practices which lead to a Phytophthora inoculum of more rigorous zoospores, or with a slightly increased titer.

### Conclusions

These data confirm that Confine, when applied as a foliar spray to the field plants during the growing season, is transported (as chemical or as signal) into the potato tubers, where it accumulates to a high enough level to provide some protection against Phytophthora infestans infection. Cultivar susceptibility to Phytophthora infestans infection decreases from Shepody to Prospect to Russet Burbank. These conclusions were supported by sporangial counts, and percent damaged tissue and percent area covered with mycelia growth.

3.3 Whole Plant Infection after Confine Treatment—Third Experiment

Since all the previous infection experiments were done by using either detached leaves or tuber slices, whole plant infection system was established in order to detect the effect of Confine. These experiments were carried out in controlled environment e.g. growth chambers, to contain the pathogen. The growth conditions and infection procedures are described in the Materials and Methods.

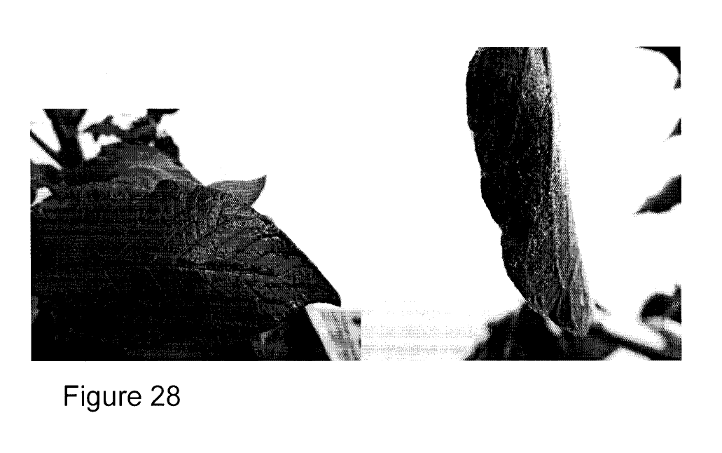

FIG. 27 showed representative views of the plants treated by Confine (1%) or water (control) from 7 days after infection to as long as 35 days after infection. The effect on late blight suppression is clearly shown from the treated plants. These plants have all survived and produced tubers at the end of their life cycle. All control plants have died before the experiments were completed. FIG. 28 demonstrated the view of localized cell death observed in Confine treated leaves as seen before, as well as the leaves form the control plants showing the massive production of the pathogen.

### 3.4 The Effect of Confine Application Rate and Frequency to Late Blight Suppression—Fourth Experiment

This experiment used whole plant grown from seed tubers (var. Shepody). Samples contain: 1) Control (untreated plants); 2) 2 treatments with 0.5% Confine (2×0.5); 3) 4 treatments with 0.5% Confine (4×0.5); 4) 2 treatments with 1% Confine (2×1); and 5) 4 treatments with 1% Confine (4×1).

Experimental details: There were 8 plants per variable (8 plants×4 variables) and 8 control plants.

Variable 1: 4 treatments with 0.5% Confine; one treatment/2 weeks. Treatments were applied on March 29, April 12, April 26 and May 10.

Variable 2. 2 treatments with 0.5% Confine; one treatment/4 weeks. Treatments were applied on March 29 and April 26.

Variable 3. 4 treatments with 1% Confine; one treatment/2 weeks. Treatments were applied on March 29, April 12, April 26 and May 10.

Variable 4. 2 treatments with 1% Confine; one treatment/4 weeks. Treatments were applied on March 29 and April 26.

Potato seeds used in the experiment were planted on February 24, plants started to emerge 2 weeks later. On May 20 (10 days after the last inoculation for 4× plants and 24 days for 2× plants), 4 plants from the four variables and 4 control plants were washed (submerged for 10-15 seconds) twice with 201 of water (in 1 20 l bucket) and sprayed with late blight sporangia.

For the pathogen infection, 10 ml of sporangial solution/plant (15,000 sporangia/ml or 150,000 sporangia/plant) was used. Plants were placed in a transparent garbage bag in the growth chamber in a light-dark cycle of 12 h/12 h and at a temperature of 15° C. The progression of late blight infection was monitored on days 4, 5, 6, 7 and 10. On day 10 plants/transparent bags/pots/bamboo sticks were sorted in different autoclavable bags and autoclaved for 40 minutes.

### Results

All treatments increased the resistance of potato plants to late blight; however there is a strong concentration effect. This effect is visible as early as on day 4.

Symptoms (necrotic spots) became visible on day 4 on all plants; however, while sporangia could be observed on day 4 on control plants, they could be identified on some 0.5% plants on day 5 and on some plants treated with 1% only on day 6. On days 6, 7 and 10 abundant sporangia production (obvious by visual inspection) was observed in all control and 2×0.5% plants; however, on many plants from the other treatments late blight sporangia production could be determined only after examination under stereomicroscope.

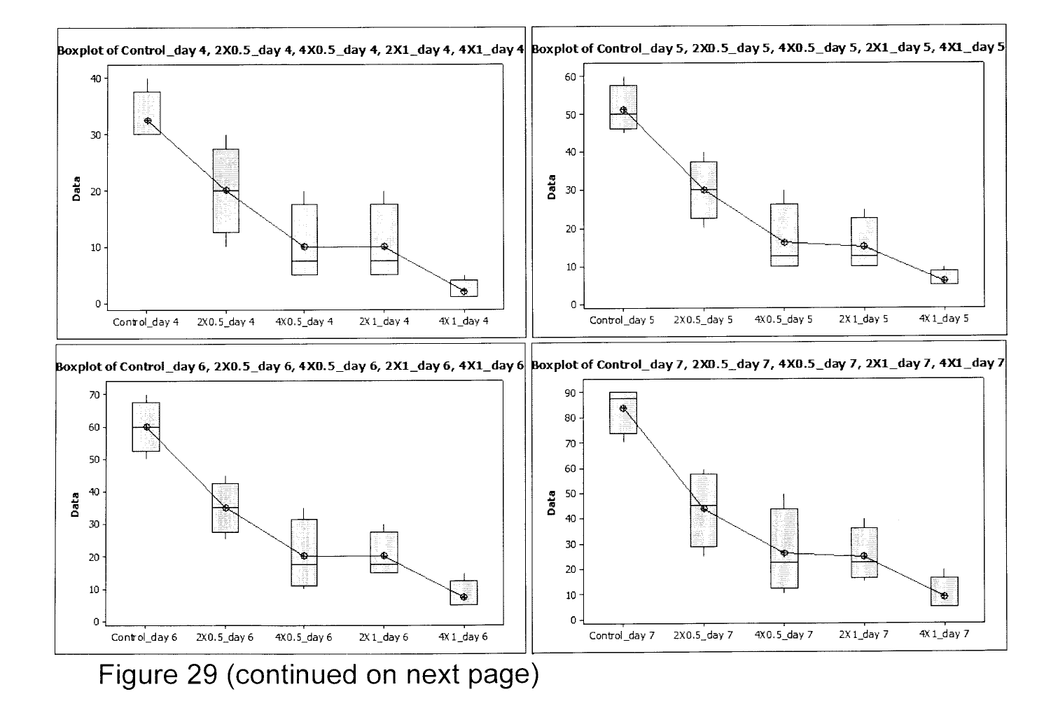

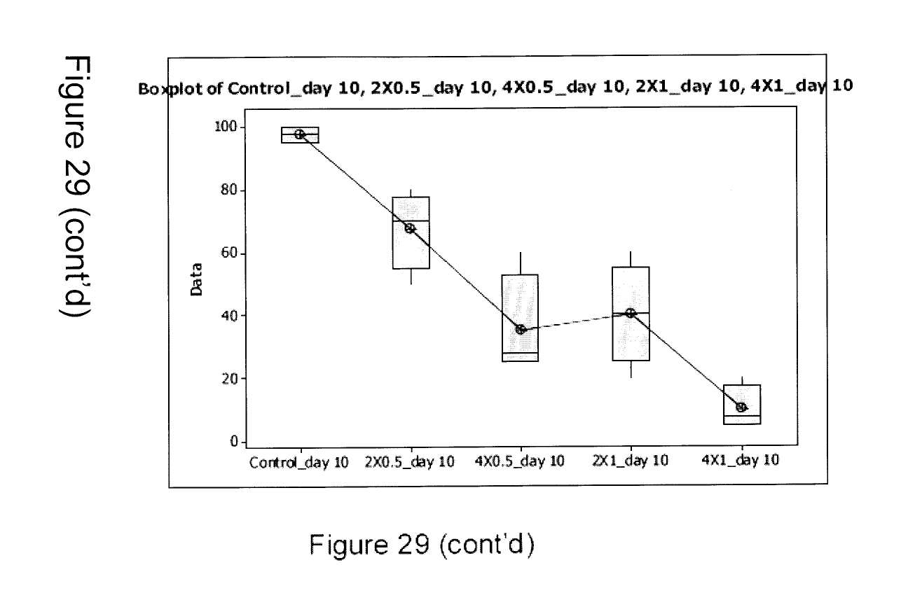

Therefore, it was concluded that under growth chamber conditions, the effect of Confine is dose dependent (0.5% or 1%), meaning that higher dosage of application (1%) will result in better protection, in this case, 1% (FIG. 29). In addition, the effect of Confine application is frequency dependent (2 times or 4 times), meaning that more frequent application results in better protection, in this case, 4 times (FIG. 29). In this series of experiments, the best Confine application combination is 1% applied 4 times. Application of 0.5% with 4 times makes the same effect as that of 1% with 2 times. Application of 0.5% twice, although provides some protection, does not give as good protection as the others.

### Materials and Methods:

### 1. Multiple Reaction Monitoring (MRM)

This method was used to validate the proteins identified by proteomics in Example 1. Total 15 proteins were examined, with 10 from the 103 proteins and 5 newly selected proteins (Table 7) based on the potential functions related to the other identified proteins.

### Materials:

Frozen leaf samples used for this experiment were from the 2007 field trial produced at Cavendish Farms, PEI. Variety Russet Burbank from Rep 2 and 3 were used in this validation.

### Method:

Liquid chromatography multiple reaction monitoring (LC-MRM) assay was used for validation of PA-responsive proteins. The extracted proteins from PA-treated and water-treated two independent biological samples (2007) grown in Cavendish Farms were digested with trypsin and labeled with mTRAQ for relative quantification. The labeled peptides (Table 8) were separated by 1D-LC-MRM-MS. Three or four transitions of each peptide for the target set of proteins were selected from identified spectra. Acquisition parameters were 50 ms dwell time and collision energy (CE)=parent m/z divided by 20. MS/MS spectra were searched against potato database using ProteinPilot with the following parameters: MMTS, mTRAQ-labeling of lysine and N-termini as fixed modifications, and a detected protein threshold of 0.05. The identified peptides were exported to MRM Pilot (v2). Matching transitions for ‘heavy’ and ‘light’ mTRAQ-labelled peptides were calculated.

### 2. Real-Time qRT-PCR

Real-time quantitative RT-PCR was also used to validate proteins initially identified.

### Materials:

Potato tuber seeds (the cultivar Shepody seed tubers) were planted in 1 gallon pots and grown in a growth chamber at under a cycle of 16 hours of light (temperature of 24±2° C.) and 8 hours of dark (16±2° C.) at NSAC. Potato seed tubers were planted on Dec. 9, 2010 and shoots start to emerge after 3 weeks (end of December). One month and a half after planting, plants reached a height of 30-50 cm and started flowering.

### Methods:

1) Confine treatment: Forty potato plants, 1.5 month old, 30-50 cm tall, were used in the experiment under the growth conditions described previously. Twenty randomly selected plants represented the controls while the other 20 were treated with 1% Confine solution. Each Confine-treated plant was sprayed with 10 ml of 1% Confine and samples were collected as described below.
2) Sampling procedure and storage of leaf samples: 1% Confine treated leaf samples were taken from 4 different plants 30 minutes post treatment (pt), 2 hours pt, 6 hours pt, 24 hours pt and 48 hours pt (4 plants/time point; 5 time points; 4 plants×5 time points=20 plants). Leaf samples were labeled and placed in aluminum foil and flash-frozen by submerging the aluminum bag in liquid nitrogen, then stored at −80° C. freezer until RNA extraction. Leaf samples for the first time point (untreated 0) of the control (untreated) plants were taken before starting spraying Confine on the Confine-treated group of plants and after 2 hours, 6 hours, 24 hours and 48 hours (4 plants/time point; 5 time points; 4 plants×5 time points=20 plants). Ten days after Confine treatment, a new set of leaf samples was taken from both Confine-treated and untreated plants. The 8 plants used for 6^{th} time point (4 plants/experimental condition; day 10 after Confine treatment) were randomly selected from the 20 plants of the Confine-treated group and from those representing the untreated group of plants.
3) Whole plant pathogen infection: 10 days after Confine treatment the 20 control and 20 Confine-treated plants were placed in transparent bags and inoculated with 10 ml of suspension (10³ sporangia/ml) of late blight (Phytophthora infestans) strain A2 US8 and the severity of disease was monitored during a 4-week period. Temperature in the growth chamber was changed to be optimal for pathogen development to 16±2° C. (16 hours of light) and 14±2° C. (8 hours of dark).
4) Leaf sampling for qPCR analysis of gene expression in plants treated with 1% Confine and infected with P. infestans. Eleven days after inoculation apparently healthy leaves and leaves with necrotic spots caused by P. infestans (foliage late blight>10% on leaves with necrotic spots) were detached from 4 randomly selected Confine-treated plants. The necrotic spots were removed by cutting at 2-3 mm from the visible spots; leaves with removed necrotic spots were placed in aluminum foil bags and submerged in liquid nitrogen. Another set of leaves that showed no signed of infection (no necrotic spots) were placed directly in aluminum foil bags and submerged in liquid nitrogen. Samples were maintained at −80° C. until processing.
5) Total RNA extraction. Leaf samples were ground in liquid nitrogen and re-suspended in the RLT buffer from the Plant RNeasy Mini Kit (Qiagen, Burlington, ON, Canada). The isolation of total RNA included the optional on-column DNase digestion step and was performed using the Plant RNeasy Mini Kit protocol according to the manufacturer's instructions. The quality and quantity of total RNA samples was assessed both by separating these samples on a 1% native agarose gel, and by using a Ultrospec 3000 spectrophotometer (Biochrom Ltd., Cambridge, England).
6) cDNA synthesis. One-step RT-PCR was performed using Ambion's RETROscript kit (Ambion/Applied Biosystems, Austin, Tex., USA) following the manufacturer's instructions. cDNA synthesis was performed at 44° C. for 60 min and then the reaction was terminated by heating the samples at 92° C. for 10 min.
7) qPCR and data analysis. The expression level of 15 gene/gene families was analyzed by qPCR using Fast SYBR Green® and the iCycler iQ5 Real-Time PCR system (Bio-Rad Laboratories, Mississauga, Ontario). qPCR primers used in the qPCR experiments are listed in Table 10. RT-PCR amplification using these primers, followed by gel electrophoresis, indicated that a single product of the correct size was obtained for each primer pair; in addition, following qPCR amplification, melt-curves (0.5° C. from 55 to 95° C.) were used to verify that a single amplicon was obtained, and that no primer-dimer products were present. qPCR was performed in a 20 μl reaction volume using 2 μl of cDNA (50 ng total RNA), 1 μl 4 μM each of forward and reverse primers and 10 μl 2× Fast SYBR Green® Master Mix. Expression levels of the target genes were normalized to the EF1-alpha gene. The decision to use EF1-alpha as the endogenous control was based on supporting literature and on in house experiments. Cycling parameters consisted of one denaturing cycle of 95° C. for 30 sec., followed by 40 cycles of 95° C. for 10 sec. and 60° C. for 30 sec. Cycled 96-well plates contained duplicate samples for each target gene together with the endogenous control. The fluorescence threshold cycle (C_{T}) was determined automatically using iQ5 iCycler's software. Transcript abundance was determined using the comparative C_{T} method for relative quantification (Livak and Schmittgen, 2001), by employing the individual with the lowest gene expression/time point as calibrator. Amplification efficiency (E=(10)^{(-1/slope)}) was assessed by running six (1:5) serial dilutions, using a starting concentration of cDNA corresponding to 50 ng of total RNA. Amplification efficiency was calculated using iQ5 iCycler's software.

### 3. PA (Confine) Application to Potato Plants Grown in a Growth Chamber for H₂O₂ Detection, LM, SEM, TEM, and Callose Deposition Analysis

Russet Burbank (RB) tuber seeds were planted in pots and grown in growth chamber at NSAC two times. Leaves were detached from 5-7 week-old RB potato plants depending on leaf size. Plants grown for LM and SEM were harvested in April, 2010. Plant grown for TEM and callose deposition were harvested in June, 2010. Growth chamber #9 was used for growing potato plants at NSAC. Temperature regimes are 22° C.-24° C. (day), 16° C.-18° C. (night), 16 hr/8 hr (day/night), humidity: 80%-90%. After pathogen infection, the detached leaves were kept in a growth chamber at NRC, temperatures were: 15° C.-18° C., 12 hr/12 hr (day/night).

Diluted Confine 400 mL (0.2 mL Confine in 10 mL water per one plant was made (8 mL confine/400 mL water) to spray 40 plants. About 1 mL was sprayed to one plant by a pre-test. Ten sprays in total were given to each plant on the leaf surface. It was ensured that a good coverage of the leaves and stem was given.

The sporangia from fresh pathogen cultures on tuber slices was counted prior to the infection using a haemocytometer under inverted microscope (1×10⁴).

Confine (PA) and water (Check)-treated leaves were detached. Total 24 leaves containing 3 time points, 4 replicates under two conditions were collected. The leaf position for each time point is; 2^{nd} leaf from bottom for 8-10 hpi (hour post infection), 3^{rd} for 36 hpi, and 4^{th} for 3 dpi (day post infection).

Infection with P. infestans (1×10⁴ sporangia in fresh distilled water) was taken place on the leaf surface by one spray (1 mL). After the infection, the leaves were incubated in each plastic bag at 15-18° C. (12 hr light/12 hr dark).

When disease symptoms were shown from 3 dpi to 6 dpi, leaves were cut to 1 cm×1 cm pieces and used for microscopy analysis.

### 4. H₂O₂ Detection by DAB Staining

H₂O₂ was visualized by staining with 3,3diaminobenzidine (DAB)-HCl. Control-, Confine-treated leaves at 3 dpi through 6 dpi (day post inoculation) were placed in 1 mg/mL DAB in 0.01M MES, pH 3.8 (Sigma, Mo., USA; #D8001). Leaves were incubated for 8 h at 16° C. and cleared in boiling ethanol (96%) for 10 min. H₂O₂ by the reaction with DAB was visualized as the deep-brown color. (Thordal-Christensen et al. 1997)

### 5. Sample Preparation for TEM

The collected leaves were treated as following before observed under the TEM. First, primary fixation took place after the potato leaves were cut as 2×2 mm or as small as possible. They were fixed with 3% glutaraldehyde in 0.1 M sodium cacodylate buffer pH 7.4 for 2 hr at room temperature, then washed two times with 0.1 M sodium cacodylate buffer pH 7.4 for 15 min. After that, the secondary fixation took place in 1.0% (w/v) osmium tetroxide in 0.1 M sodium cacodylate buffer pH 7.4 for 1.5 hr at room temperature. When it was completed, the leaves were washed two times with 0.1 M sodium cacodylate buffer pH 7.4 for 15 min. Finally, the leaves were dehydrated step by step using 30%, 50%, 75%, 85%, and 95% ethanol for 15 min each time. They were then dehydrated again three times with 100% ethanol for 15 min each, then with 100% acetone for 15 min. After that, they were infiltrated using 1:3 epon resin:acetone (5 ml:10 ml) for 2-4 hrs, 1:1 epon resin:acetone (7.5 ml:7.5 ml) for 1-3 hrs, and 3:1 epon resin:acetone (10 ml: 5 ml) for overnight at room temperature. The final polymerization step took place in 100% epon resin for overnight at 60° C.

The observations took place under light microscopy (DMRE LIECA) by 1 mm semithins (Reichert ultracuts). Pictures were captured by simple PCI software. The observations under TEM (Hitachi 7500) were by 80 nm ultrathins (Reichert ultracuts). Pictures were taken by Bioscan camera with Gatan software.

### 6. Callose Deposition Analysis

For microscopic examination of callose deposition on potato leaves, infected water-treated (control) and Confine-treated potato leaf samples were re-hydrated through a gradual decrease of ethanol of 100%, 80%, 70%, and 50%. Samples were stained in 0.05% aniline blue in 0.15 M KH₂PO₄, pH 9.5 overnight and then de-stained in 0.15 M KH₂PO₄, pH 9.5. De-stained samples were mounted in 30% glycerol on glass slides and were examined using a UV epifluorescence microscope (DMRE, Leica Wetzler, Germany). Autofluorescence was visualized with a 450-490 nm BP excitation filter and a 515 nm LP emission filter. For callose observation, excitation filter BP 340-380 nm and emission filter LP 425 nm were used. Light microscope was used to examine infected area. All images were processed using the software, Compix Simple PCI (JH Technologies, San Jose, USA).

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1A
Proteins up-regulated by PA. The first 72 proteins were identified by protein profiling. The last 12 proteins were
identified by MRM or qPCR. Accession numbers, annotation and functional classification of each protein are given. The
proteins from protein profiling were isolated either form the cell wall (wall) or cytoplasm (cyto) fractions. Dyes 114, 115, 116
and 117 were used to label the four individual groups of samples.

| | | | | | | | | Classification |
|---|---|---|---|---|---|---|---|---|
| Accession | Genbank | | | | | | | by Bevan et |
| no | no. | Annotation | UniRef100 | fraction | 115/114 | 116/114 | 117/114 | al. (1998) |
| TC176356 | CK263124 | Basic PR-1 protein | Q04106 | wall | 2.84 | 11.88 | 11.02 | Defense |
| TC169479 | BQ121995 | Basic PR-1 protein | O65157 | cyto | 1.81 | 9.97 | 12.38 | Defense |
| TC169479 | BQ121995 | Basic PR-1 protein | O65157 | wall | 3.32 | 14.55 | 10.72 | Defense |
| TC168375 | AM906471 | PR-1 Protein | Q9SC15 | wall | 1.55 | 4.98 | 4.23 | Defense |
| TC170396 | AM906608 | Pathogenesis related protein isoform | Q8L688 | cyto | 1.68 | 4.84 | 7.33 | Defense |
| | | b1 | | | | | | |
| TC170396 | AM906608 | Pathogenesis related protein isoform | Q8L688 | wall | 1.83 | 6.31 | 5.27 | Defense |
| | | b1 | | | | | | |
| TC172275 | AM906826 | Pathogenesis-related protein P2 (PR4) | P32045 | cyto | 1.79 | 6.75 | 7.36 | Defense |
| TC172275 | AM906826 | Pathogenesis-related protein P2 | P32045 | wall | 1.63 | 2.44 | 2.86 | Defense |
| TC173865 | AJ009932 | Beta-1,3 glucanase | O82063 | cyto | 1.82 | 2.81 | 2.49 | Defense |
| TC163195 | AJ586575 | Beta-1,3 glucanase | Q70C53 | cyto | 1.82 | 2.62 | 2.89 | Defense |
| TC175030 | CV503143 | Beta-1 3-1 4-glucanase | Q9LUG8 | wall | 1.43 | 0.83 | 0.71 | Defense |
| TC168318 | CK261561 | Class II chitinase | Q43834 | cyto | 1.46 | 2.50 | 2.05 | Defense |
| TC168318 | CK261561 | Class II chitinase | Q43834 | wall | 1.41 | 2.56 | 1.34 | Defense |
| CK263954 | CK263954 | Class II chitinase | Q43835 | cyto | 1.48 | 2.17 | 2.41 | Defense |
| CK263954 | CK263954 | Class II chitinase | Q43835 | wall | 1.53 | 2.65 | 1.44 | Defense |
| TC168794 | CK248321 | Class V Chitinase | Q43576 | cyto | 1.41 | 5.08 | 4.61 | Defense |
| TC163769 | CK266830 | Acidic endochitinase | P29060 | cyto | 1.44 | 2.99 | 1.81 | Defense |
| TC163769 | CK266830 | Acidic endochitinase | P29060 | wall | 1.58 | 1.45 | 1.02 | Defense |
| TC163429 | CK273245 | Endochitinase | Q43184 | wall | 1.44 | 1.61 | 1.40 | Defense |
| TC189821 | CK640790 | Pathogenesis-related protein R major | P13046 | wall | 1.82 | 2.12 | 1.75 | Defense |
| | | form | | | | | | |
| TC169893 | AM906485 | Thaumatin-like protein | Q5XUG9 | wall | 1.63 | 1.98 | 1.67 | Defense |
| TC172434 | DN941054 | Peroxidase | Q9LWA2 | cyto | 1.52 | 2.38 | 2.59 | Defense |
| TC172434 | DN941054 | Peroxidase | Q9LWA2 | wall | 1.43 | 1.71 | 1.62 | Defense |
| TC169870 | CV430433 | Peroxidase | Q43774 | cyto | 1.73 | 2.61 | 3.00 | Defense |
| TC164504 | CK246202 | Peroxidase | Q50LG4 | wall | 1.71 | 5.75 | 3.00 | Defense |
| TC186921 | BQ515006 | Glutathione S-transferase | A5YWI8 | cyto | 1.50 | 1.84 | 1.83 | Defense |
| TC186921 | BQ515006 | Glutathione S-transferase | A5YWI8 | wall | 1.46 | 2.02 | 1.28 | Defense |
| TC177499 | CK718853 | Glutathione S-transferase | Q76KW1 | cyto | 1.41 | 1.74 | 1.79 | Defense |
| TC176436 | BG350528 | Monodehydroascorbate reductase | Q43497 | wall | 1.41 | 1.45 | 1.19 | Defense |
| TC186221 | CK261937 | Putative heat shock protein | Q9SR91 | wall | 1.63 | 1.02 | 2.29 | Defense |
| TC182291 | AM907618 | Osmotin-like protein OSML81 | P50702 | wall | 1.72 | 15.27 | 11.23 | Defense |
| TC165487 | CK243755 | Osmotin-like protein | Q5XUH6 | cyto | 1.48 | 10.39 | 11.39 | Defense |
| TC165487 | CK243755 | Osmotin-like protein | Q5XUH6 | wall | 1.70 | 13.10 | 9.28 | Defense |
| TC163374 | CK246914 | Osmotin-like protein | Q5XUH5 | wall | 1.99 | 36.58 | 33.35 | Defense. |
| TC171679 | BI434095 | Osmotin-like protein | Q5XUH3 | wall | 1.56 | 13.62 | 9.88 | Defense |
| TC179738 | BG597229 | GDSL-motif lipase/hydrolase-like | Q9FLN0 | wall | 1.40 | 0.42 | 1.01 | Defense |
| | | protein | | | | | | |
| TC189750 | X13497 | Wound-induced protein WIN1 | P09761 | wall | 1.49 | 13.17 | 13.63 | Defense |
| TC182527 | CV474562 | Wound-induced proteinase inhibitor 1 | P08454 | cyto | 1.44 | 1.24 | 2.14 | Defense |
| TC173874 | BQ047477 | Germin-like protein | Q0PWM4 | cyto | 1.48 | 3.56 | 2.09 | Defense |
| TC176976 | BG887746 | Cathepsin B | Q1HER6 | cyto | 1.39 | 2.15 | 2.40 | Defense |
| TC172593 | CK263045 | Cysteine proteinase 3 | Q40143 | cyto | 1.45 | 2.04 | 1.93 | Defense |
| TC181645 | CX161936 | Cysteine protease inhibitor 9 or 1 | Q00652 | wall | 1.79 | 0.55 | 0.63 | Defense |
| TC166886 | CV431636 | Cysteine protease inhibitor 7 | O24385 | wall | 2.04 | 0.51 | 0.73 | Defense |
| TC166762 | CV503853 | Cysteine protease inhibitor 1 | P20347 | cyto | 1.46 | 0.70 | 1.12 | Defense |
| TC166762 | CV503853 | Cysteine protease inhibitor 1 | P20347 | wall | 1.55 | 0.64 | 0.56 | Defense |
| TC186600 | EG012236 | 60S acidic ribosomal protein | Q307Z8 | cyto | 1.78 | 1.11 | 1.52 | Protein |
| | | | | | | | | synthesis |
| TC186583 | BQ046290 | Ribosome recycling factor | P82231 | cyto | 1.58 | 1.06 | 1.12 | Protein |
| | | | | | | | | synthesis |
| TC180423 | BG589296 | Acidic ribosomal protein P1a-like | Q307Z7 | cyto | 1.40 | 2.15 | 1.47 | Protein |
| | | | | | | | | synthesis |
| TC178608 | CN214527 | 40S ribosomal protein S16 | Q9M5L1 | cyto | 1.44 | 1.51 | 2.31 | Protein |
| | | | | | | | | synthesis |
| TC165455 | BG599224 | 60S ribosomal protein L13 | Q3HRY7 | cyto | 1.68 | 1.95 | 1.70 | Protein |
| | | | | | | | | synthesis |
| TC165221 | CK276504 | 30S ribosomal protein S9 | Q9XJ27 | cyto | 2.14 | 0.50 | 0.73 | Protein |
| | | | | | | | | synthesis |
| TC163292 | CK270139 | Elongation factor 1-alpha | Q2V985 | cyto | 1.40 | 1.98 | 1.79 | Protein |
| | | | | | | | | synthesis |
| CV470531 | CV470531 | Elongation factor TuB | Q43364 | cyto | 1.47 | 1.21 | 1.13 | Protein |
| | | | | | | | | synthesis |
| TC166345 | DN923394 | Biotin carboxylase | Q40475 | cyto | 1.44 | 0.72 | 1.05 | Metabolism - |
| | | | | | | | | fatty acid |
| | | | | | | | | biosynthesis |
| TC183342 | CK278159 | 3-ketoacyl-CoA thiolase 2 | Q56WD9 | cyto | 1.76 | 4.20 | 3.38 | Metabolism - |
| | | | | | | | | fatty acid |
| | | | | | | | | degradation |
| TC165530 | CK264757 | Adenylyl-sulfate reductase | Q672Q8 | cyto | 1.52 | 0.25 | 0.36 | Metabolism - |
| | | | | | | | | cysteins, |
| | | | | | | | | glutathione |
| TC165467 | CK269790 | Pyrroline-5-carboxylate reductase | O04016 | cyto | 1.45 | 1.67 | 2.15 | Metabolism - |
| | | | | | | | | proline to |
| | | | | | | | | glutamate |
| TC163648 | Z25863 | Serine hydroxymethyltransferase | P50433 | cyto | 1.43 | 1.33 | 0.98 | Metabolism - |
| | | | | | | | | serine to |
| | | | | | | | | glycine |
| TC194216 | CK275814 | Glyceraldehyde-3-phosphate | P12859 | wall | 1.40 | 0.59 | 0.78 | Energy - |
| | | dehydrogenase | | | | | | glycolysis |
| TC166486 | CK271926 | Glyceraldehyde-3-phosphate | P09044 | wall | 1.72 | 0.75 | 0.87 | Energy - |
| | | dehydrogenase | | | | | | glycolysis |
| TC181183 | CK267911 | NADP-dependent glyceraldehyde-3- | P93338 | wall | 1.47 | 1.36 | 1.00 | Energy - |
| | | phosphate dehydrogenase | | | | | | glycolysis |
| TC163506 | DQ200399 | Triosephosphate isomerase | Q3HRV9 | wall | 1.56 | 0.81 | 0.64 | Energy - |
| | | | | | | | | glycolysis |
| TC165027 | DN922879 | Succinyl-CoA ligase | Q8GTQ9 | wall | 2.27 | 2.90 | 2.02 | Energy - TCA |
| TC172096 | AM908584 | Histidine triad (HIT) protein | Q2MGQ9 | cyto | 1.43 | 0.89 | 1.16 | Signal |
| | | | | | | | | transduction |
| TC168258 | J04559 | Calmodulin | P27161 | cyto | 2.06 | 1.23 | 1.81 | Signal |
| | | | | | | | | transduction |
| TC166307 | DQ200375 | Calmodulin-5/6/7/8 | Q7DMN9 | cyto | 1.40 | 1.23 | 1.58 | Signal |
| | | | | | | | | transduction |
| TC165237 | CK269076 | BTF3-like transcription factor | Q2PQI9 | cyto | 1.46 | 1.17 | 1.19 | Signal |
| | | | | | | | | transduction |
| TC180805 | BI177238 | Putative uncharacterized protein | O60732 | wall | 2.20 | 1.68 | 0.65 | Unknown |
| TC168485 | CK269423 | Putative uncharacterized protein | A7P413 | cyto | 1.43 | 1.28 | 1.45 | Unknown |
| TC164517 | CK269189 | Putative uncharacterized protein | A7QVC3 | cyto | 2.15 | 0.66 | 0.66 | Unknown |
| TC164403 | CK258232 | Pyutative uncharacterized protein | Q38HV9 | cyto | 1.40 | 1.53 | 1.75 | Unknown |
| TC167954 | CV286818 | Aminopeptidase | Q9CAE1 | wall | 1.90 | 0.86 | 0.83 | Protein |
| | | | | | | | | destination |
| TC166277 | CK270757 | Peroxidase - New/MRM | | | | | | Defense |
| TC183478 | CK264126 | Beta-1,3-glucanase - New/MRM | | | | | | Defense |
| TC169550 | DN923375 | Cysteine protease inhibitor - New/MRM | | | | | | Defense |
| CK276749 | CK276749 | Subtilisin - New/MRM | | | | | | Defense |
| CK161954 | CK161954 | Aspartic protease inhibitor 10 - | | | | | | Defense |
| | | New/MRM | | | | | | |
| | CAD61188 | Sucrose synthase 4 - New/qPCR | | | | | | starch and |
| | | | | | | | | sucrose |
| | | | | | | | | metabolism |
| | Y08786 | 1,4-alpha-glucan branching enzyme - | | | | | | starch and |
| | | New/qPCR | | | | | | sucrose |
| | | | | | | | | metabolism |
| | DQ235169 | Fructose-bisphosphate aldolase-like - | | | | | | glycolysis and |
| | | New/qPCR | | | | | | ancillary |
| | | | | | | | | pathways |
| | BF460265 | Mitochondrial ATP synthase subunit | | | | | | energy |
| | | beta - New/qPCR | | | | | | metabolism, |
| | | | | | | | | ATP synthesis |
| | DQ386163 | Chloroplastidial ATP synthase subunit | | | | | | energy |
| | | alpha - New/qPCR | | | | | | metabolism, |
| | | | | | | | | ATP synthesis |
| | DQ231562, | Chloroplastidial ATP synthase subunit | | | | | | energy |
| | YP_635646 | beta - New/qPCR | | | | | | metabolism, |
| | | | | | | | | ATP synthesis |
| | AJ009932, | Beta 1,3 glucanase (many members of | | | | | | starch and |
| | AY170826 | the gene family) - New/qPCR | | | | | | sucrose |
| | | | | | | | | metabolism |

TABLE 1B
Proteins down-regulated by PA. The first 31 proteins were identified by protein profiling. The last protein was
identified by qPCR. Accession numbers, annotation and functional classification of each protein are given. The proteins
from protein profiling were isolated either form the cell wall (wall) or cytoplasm (cyto) fractions. Dyes 114, 115,
116 and 117 were used to label the four individual groups of samples.

| Accession | Genbank | | | | | | | Classification by |
|---|---|---|---|---|---|---|---|---|
| no | no. | Annotation | UniprotKB | Fraction | 115/114 | 116/114 | 117/114 | Bevan et al. (1998) |
| CK257172 | CK257172 | Pyruvate | P52903 | wall | 0.69 | 0.45 | 0.64 | Energy - TCA |
| | | dehydrogenase | | | | | | |
| TC171649 | BG589586 | Pyruvate kinase | Q1SN32 | cyto | 0.58 | 1.19 | 1.46 | Energy - Glycolysis |
| TC168267 | CK278107 | Fructose-bisphosphate | Q9SXX4 | wall | 0.61 | 0.47 | 0.45 | Energy - Glycolysis |
| | | aldolase | | | | | | |
| TC164121 | EU344848 | Fructose-bisphosphate | Q9SXX5 | wall | 0.67 | 0.51 | 0.44 | Energy - Glycolysis |
| | | aldolase | | | | | | |
| TC163071 | AY690423 | Glucose-6-phosphate | Q68HC9 | cyto | 0.67 | 0.96 | 1 | Energy - Glycolysis |
| | | isomerase | | | | | | |
| TC167284 | CK267355 | 6-phosphogluconate | Q8LG34 | cyto | 0.71 | 0.78 | 1.08 | Energy - pentose |
| | | dehydrogenase | | | | | | phosphate |
| TC191617 | CK263234 | Phosphoribulokinase | A5BE19 | wall | 0.71 | 0.86 | 0.87 | Energy - reductive |
| | | | | | | | | phosphate |
| TC163042 | X67053 | Phosphoenolpyruvate | P29196 | cyto | 0.5 | 0.88 | 0.89 | Metabolism - |
| | | carboxylase | | | | | | photosynthesis |
| | | | | | | | | (carbohydrate) |
| TC181436 | CK276155 | Phosphoenolpyruvate | A7UH66 | cyto | 0.64 | 1.05 | 0.99 | Metabolism - |
| | | carboxylase | | | | | | photosynthesis |
| | | | | | | | | (carbohydrate) |
| TC164001 | CK276445 | Oxygen-evolving | P23322 | wall | 0.65 | 0.4 | 0.33 | Metabolism - |
| | | enhancer protein | | | | | | photosynthesis |
| | | | | | | | | (carbohydrate) |
| TC163384 | CV286411 | Oxygen-evolving | P26320 | wall | 0.75 | 0.48 | 0.36 | Metabolism - |
| | | enhancer protein 1 | | | | | | photosynthesis |
| | | | | | | | | (carbohydrate) |
| TC179073 | BI433195 | Ferredoxin | Q6Q8B8 | wall | 0.63 | 0.64 | 0.5 | Metabolism - |
| | | | | | | | | photosynthesis |
| | | | | | | | | (carbohydrate) |
| TC165919 | M69038 | Alpha-glucan | P32811 | cyto | 0.41 | 0.86 | 1 | Metabolism - starch |
| | | phosphorylase | | | | | | degradation |
| TC165690 | X52385 | Alpha-glucan | P04045 | cyto | 0.72 | 0.67 | 0.73 | Metabolism - starch |
| | | phosphorylase | | | | | | degradation |
| TC163028 | Y09533 | Alpha-glucan water | Q9AWA5 | cyto | 0.64 | 0.56 | 0.8 | Metabolism - starch |
| | | dikinase | | | | | | degradation |
| TC163054 | AY205084 | Sucrose synthase 2 | Q84UC3 | cyto | 0.53 | 2.19 | 1.97 | Metabolism - starch |
| | | | | | | | | biosynthesis |
| TC169318 | BG591371 | Glutamate synthase | Q7M242 | cyto | 0.46 | 0.55 | 0.57 | Metabolism - |
| | | | | | | | | glutamin to |
| | | | | | | | | glutamate |
| TC164369 | CK278676 | Alanine | Q9S7E9 | cyto | 0.72 | 0.48 | 0.47 | Metabolism - |
| | | aminotransferase | | | | | | alanine to glutamate |
| TC163367 | CK065278 | Aminotransferase 2 | Q6V1W4 | cyto | 0.74 | 0.58 | 0.55 | Metabolism - |
| | | | | | | | | alanine to glutamate |
| TC163226 | Z99770 | Glycine dehydrogenase | O49954 | cyto | 0.69 | 0.6 | 0.59 | Metabolism - |
| | | | | | | | | glycine to glyoxylate |
| TC190989 | BI433419 | Thaumatin-like protein | Q5ND92 | cyto | 0.61 | 1.41 | 0.92 | Defense |
| TC189821 | CK640790 | Pathogenesis-related | P13046 | cyto | 0.63 | 1.87 | 0.98 | Defense |
| | | protein R major form | | | | | | |
| TC165098 | DN938857 | Glutathione S- | Q9FT19 | cyto | 0.75 | 0.97 | 1.15 | Defense |
| | | transferase | | | | | | |
| TC172573 | BG600110 | Polygalacturonase | Q6BDJ1 | wall | 0.65 | 0.48 | 0.88 | Defense |
| | | inhibitor protein | | | | | | |
| TC181534 | CK278403 | Elongation factor EF-2 | Q9SGT4 | cyto | 0.62 | 0.94 | 1.22 | Protein synthesis |
| TC164483 | CK263568 | Ribosomal protein L3- | Q2VCJ2 | cyto | 0.72 | 0.72 | 0.78 | Protein synthesis |
| | | like | | | | | | |
| TC169973 | DR035917 | 40S ribosomal protein | Q9XHS0 | wall | 0.74 | 0.39 | 0.7 | Protein synthesis |
| | | S12 | | | | | | |
| EG015239 | EG015239 | Putative | Q01JN2 | wall | 0.61 | 0.06 | 1.02 | Unknown |
| | | uncharacterized protein | | | | | | |
| EG016190 | EG016190 | Putative | A5BVQ5 | wall | 0.72 | 0.89 | 0.86 | Unknown |
| | | uncharacterized protein | | | | | | |
| TC175288 | BG890355 | Proteasome subunit | Q9XG77 | wall | 0.72 | 0.53 | 0.76 | Protein destination |
| | | alpha type-6 | | | | | | |
| TC164185 | CK269415 | Histone H1 | O65820 | wall | 0.74 | 0.75 | 0.74 | Transcription |
| | X73684 | Alpha glucan | New/qPCR | | | | | starch and sucrose |
| | | phosphorylase type L2 | | | | | | metabolism |

TABLE 2
The 45 proteins in defense category

| Annotation | # | Annotation | # |
|---|---|---|---|
| Basic PR-1 protein | 6 | Putative heat shock protein | 1 |
| Beta-1,3 glucanase | 2 | Osmotin-like protein | 4 |
| Beta-1,3-1,4-glucanase | 1 | Wound-induced protein WIN1 | 1 |
| Class II chitinase | 3 | Wound-induced proteinase inhibitor | 1 |
| Acidic endochitinase | 2 | Germin-like protein | 1 |
| Thaumatin-like protein | 1 | Cysteine proteinase | 1 |
| Peroxidase | 3 | Cysteine protease inhibitor | 3 |
| Glutathione S-transferase | 2 | Cathepsin B | 1 |
| Monodehydroascorbate | 1 | GDSL-motif lipase/hydrolase- | 1 |
| reductase | | like protein | |

#: Numbers of proteins identified in this study

TABLE 3
PA-responsive proteins when PA-treated and non-treated samples
were compared. 114/115: Control 0 day before inoculation/PA 0 day days
before inoculation. (Fold Change > 1.4)

| Proteins | Fraction | 114/115 ave |
|---|---|---|
| β-1,3 glucanase | wall | 1.8 |
| PR-1 | cyto | 1.8 |
| PR-1 | wall | 3.3 |
| GST | wall | 1.5 |
| Chitinase | cyto | 1.5 |
| Chitinase | wall | 1.4 |
| Peroxidase | cyto | 1.5 |
| Peroxidase | wall | 1.4 |
| HSP | wall | 1.6 |
| Osmotin | cyto | 1.7 |
| Cysteine protease | cyto | 1.4 |
| CP inhibitor | wall | 1.8 |

TABLE 4
PA-responsive proteins in response to P. infestans. 114: Control 0
days before inoculation; 115: PA-treated 0 days before inoculation; 116:
Control 4 days after inoculation; 117: PA-treated 4 days after
inoculation. (Fold Change > 1.4)

| Proteins | Fraction | 114/115 ave | 114/116 | 114/117 |
|---|---|---|---|---|
| β-1,3 glucanase | wall | 1.8 | 2.8 | 2.5 |
| PR-1 | cyto | 1.8 | 10.0 | 12.4 |
| PR-1 | wall | 3.3 | 14.5 | 10.7 |
| GST | wall | 1.5 | 1.8 | 1.8 |
| Chitinase | cyto | 1.5 | 2.5 | 2.1 |
| Chitinase | wall | 1.4 | 2.6 | 1.3 |
| Peroxidase | cyto | 1.5 | 2.4 | 2.6 |
| Peroxidase | wall | 1.4 | 1.7 | 1.6 |
| HSP | wall | 1.6 | 1.0 | 2.3 |
| Osmotin | cyto | 1.7 | 15.3 | 11.2 |
| Cysteine protease | cyto | 1.4 | 2.0 | 1.9 |
| CP inhibitor | wall | 1.8 | 0.5 | 0.6 |

TABLE 5
Other inhibitors

| Annotation | Accession # | 114/115 | 114/116 | 114/117 |
|---|---|---|---|---|
| Cysteine protease inhibitor 4 | TC169550 | 1.3 | 0.7 | 0.7 |
| Aspartic protease inhibitor 10 | CX161954 | 1.2 | 0.5 | 0.5 |
| Aspartic protease inhibitor 1 | TC194204 | 1.1 | 0.4 | 0.7 |
| Kunitz-type protease inhibitor | TC188516 | 1.2 | 0.4 | 0.5 |
| Proteinase inhibitor 2 | TC180217 | 0.9 | 0.6 | 0.5 |
| Wound-induced proteinase | TC190827 | 1.2 | 0.7 | 1.5 |
| inhibitor 1 | | | | |
| Polygalacturonase inhibitor | TC174920 | 1.0 | 0.9 | 1.1 |
| Proteinase inhibitor 1 | TC183941 | 1.3 | 1.3 | 1.5 |
| Ethylene-responsive proteinase | TC188093 | | 9.3 | 7.0 |
| inhibitor 1 | | | | |
| Ethylene-responsive proteinase | TC184729 | | 11.0 | 7.9 |
| inhibitor 1 | | | | |
| Kunitz-type protease inhibitor | TC169394 | | 34.2 | 14.8 |

TABLE 6
Putative function of defense proteins

| Annotation | Putative function |
|---|---|
| Basic PR-1 protein | SA signaling |
| Beta-1,3 glucanase | SA signaling |
| Beta-1,3-1,4-glucanase | degrade fungal cell walls |
| Class II chitinase | SA signaling, degrade fungal |
| | cell walls |
| Acidic endochitinase | SA signaling, degrade fungal |
| | cell walls |
| Thaumatin-like protein | SA signaling |
| Peroxidase | ROS, lignification |
| Glutathione S-transferase | ROS, SA signaling |
| Monodehydroascorbate reductase | ROS |
| Putative heat shock protein | Stress |
| Osmotin-like protein | Stress |
| Wound-induced protein WIN1 | Stress |
| Wound-induced proteinase inhibitor 1 | Stress |
| Germin-like protein | HR, degrade fungal cell walls |
| Cysteine proteinase 3 | HR, degrade fungal cell walls |
| Cysteine protease inhibitor 9 | HR, degrade fungal cell walls |
| GDSL-motif lipase/hydrolase- | HR, degrade fungal cell walls |
| like protein | |
| Cathepsin B | HR, degrade fungal cell walls |

TABLE 7
The 15 proteins validated by MRM. The first 8 proteins are
selected from the 72 up-regulated proteins' list (shown as Up);
2 proteins are selected from the 30 down-regulated proteins' list (shown
as Down). Another 5 newly selected proteins are shown as New.

| Accession | Genbank | | | Up/ |
|---|---|---|---|---|
| no. | no. | Annotation | UniRef100 | Down |
| TC168375 | | PR-1 Protein | Q9SC15 | Up |
| TC172275 | AM906826 | Pathogenesis-related | P32045 | Up |
| | | protein P2 | | |
| TC163195 | AJ586575 | Beta-1,3 glucanase | Q70C53 | Up |
| TC163769 | CK266830 | Acidic endochitinase | P29060 | Up |
| TC163429 | CK273245 | Endochitinase | Q43184 | Up |
| TC165487 | CK243755 | Osmotin-like protein | Q5XUH6 | Up |
| TC163648 | Z25863 | Serine hydroxy- | P50433 | Up |
| | | methyltransferase | | |
| TC168258 | J04559 | Calmodulin | P27161 | Up |
| TC164121 | EU344848 | Fructose-bisphosphate | Q9SXX5 | Down |
| | | aldolase | | |
| TC163226 | Z99770 | Glycine dehydrogenase | O49954 | Down |
| TC166277 | CK270757 | Peroxidase | | New/Up |
| TC183478 | CK264126 | Beta-1,3-glucanase | | New/Up |
| TC169550 | DN923375 | Cysteine protease | | New/Up |
| | | inhibitor | | |
| CK276749 | CK276749 | Subtilisin | | New/Up |
| CK161954 | CK161954 | Aspartic protease | | New/Up |
| | | inhibitor 10 | | |

TABLE 8
The 15 proteins validated by MRM. Shown are peptide used for the
detection, TC number of each protein, average intensity (Ave), standard
error (SE), and standard deviation (STDEV). Columns 4, 7, 8, 11 and 12 are
the 5 new proteins tested.

| | | | SEQ | | | | | |
|---|---|---|---|---|---|---|---|---|
| | | | ID | | | | | |
| | Q1 | Peptide | NO. | Protein | TC number | Ave | SE | STDEV |
| 1 | 999.0 | VSTSTYSGLLTNTYPPR | 1 | Beta-1,3-glucanase (TC163195) | TC163195-1 | 4.4 | 0.7 | 2.0 |
| | 715.5 | YIAVGNEVDPGR | 2 | Beta-1,3-glucanase (TC163195) | TC163195-2 | 2.7 | 0.2 | 0.6 |
| 2 | 532.4 | AQNYANSR | 3 | PR-1 (TC168375) | TC168375-1 | 3.5 | 0.5 | 1.3 |
| | 468.8 | GSGDFTGR | 4 | PR-1 (TC168375) | TC168375-2 | 2.8 | 0.3 | 0.8 |
| 3 | 752.5 | VTNTGTGTQETVR | 5 | PR P2 (TC172275) | TC172275 | 3.0 | 0.5 | 1.1 |
| 4 | 615.3 | AESIVQSTVR | 6 | Peroxidase(TC166277) | TC166277 | 2.3 | 0.2 | 0.6 |
| 5 | 658.3 | WSPSAADSAAGR | 7 | Endochitinase (TC163429) | TC163429 | 2.3 | 0.3 | 0.8 |
| 6 | 473.3 | ELGTVMR | 8 | Calmodulin (TC168258) | TC168258 | 2.3 | 0.4 | 0.9 |
| 7 | 608.3 | IGQMTQIER | 9 | Beta-1,3-glucanase (TC183478) | TC183478 | 2.2 | 0.2 | 0.6 |
| 8 | 493.6 | DIHGDILTPDSR | 10 | Cysteine p inhibitor(TC169550) | TC169550 | 2.2 | 0.1 | 0.2 |
| 9 | 641.4 | GQTWVINAPR | 11 | Osmotin (TC165487) | TC165487 | 2.1 | 0.2 | 0.6 |
| 10 | 567.3 | ALSGFSQQR | 12 | Endochitinase (TC163769) | TC163769 | 2.0 | 0.2 | 0.5 |
| 11 | 811.9 | TVTNVGDATSSYK | 13 | Subtilisin(CK276749) | CK276749-1 | 1.7 | 0.2 | 0.5 |
| | 632.3 | LGSTPQTYTR | 14 | Subtilisin(CK276749) | CK276749-2 | 1.6 | 0.1 | 0.3 |
| 12 | 744.9 | YNSDVGPSGTPVR | 15 | Aspartic P inhibitor | CK161954 | 1.7 | 0.2 | 0.5 |
| | | | | 10(CK161954) | | | | |
| 13 | 545.4 | AYQEQVLSNSSK | 16 | Serine hydroxymethyltransferase | TC163648 | 1.2 | 0.1 | 0.3 |
| | | | | (TC163648) | | | | |
| 14 | 733.4 | YTGEGESDEAK | 17 | Fructose aldolase (TC164121) | TC164121 | 0.8 | 0.0 | 0.1 |
| 15 | 539.3 | VDNVYGDR | 18 | Glycine dehydrogenase | TC163226 | 0.8 | 0.1 | 0.1 |
| | | | | (TC163226) | | | | |

TABLE 9
The summarized functions of the 15 validated proteins.

| | | Relative | | | |
|---|---|---|---|---|---|
| | Protein | abundance | Putative functions | | |
| 1 | PR-1 (TC168375) | up | PR-1 (pathogenesis- | Antifungal activity | SA signalling |
| | | | related) | | |
| 2 | Beta-1,3-glucanase (TC163195) | up | PR-2 | Antifungal activity | SA signalling |
| 3 | Beta-1,3-glucanase (TC183478) | up | PR-2 | Antifungal activity | SA signalling |
| 4 | Endochitinase (TC163429) | up | PR-3 | Antifungal activity | SA signalling |
| 5 | Endochitinase (TC163769) | up | PR-3 | Antifungal activity | SA signalling |
| 6 | PR P2 (TC172275) (PR-4) | up | PR-4 | Antifungal activity | |
| 7 | Osmotin (TC165487) | up | PR-5 | Antifungal activity | SA signalling |
| 8 | Peroxidase (TC166277) | up | PR-9 | | |
| 9 | Subtilisin (CK276749) | up | protease | hypersensitive response (HR) | |
| 10 | Aspartic p inhibitor | up | protease inhibitor | HR | |
| | 10(CK161954) | | | | |
| 11 | Cysteine p inhibitor (TC169550) | up | protease inhibitor | HR | |
| 12 | Calmodulin (TC168258) | up | signal transduction | Signalling, production of ROS, HR | |
| 13 | Serine hydroxymethyl | up | Metabolism—serine to | | |
| | transferase (TC163648) | | glycine | | |
| 14 | Glycine dehydrogenase | down | Metabolism—glycine to | glyoxylate cycle is a different type of | |
| | (TC163226) | | glyoxylate | TCA cycle | |
| 15 | Fructose aldolase (TC164121) | down | Energy—Glycolysis | | |

TABLE 10
Fifteen genes analyzed by qRT-PCR in the Confine-treated plants. Function, primer sequence and
GenBank accession ID are listed. Elongation factor 1-α is used as an internal reference gene.
The 7 proteins in bold are chosen from our proteomic profiling list.

| | | | | | SEQ | |
|---|---|---|---|---|---|---|
| | | | | | ID | GenBank |
| Gene name | Abbrev. | EC | Function/metabolic pathway | Primers | NO: | # |
| Sucrose synthase 2 | SS2 | 2.4.1.13 | starch and sucrose metabolism | SS2F: TTGAAGGACAGGAATAAGCCC | 19 | AY205084 |
| | | | | SS2R: CCCTTAGTCGTGGATTCTTAGCA | 20 | |
| Sucrose synthase 4 | SS4 | 2.4.1.13 | starch and sucrose metabolism | SS4F: TGAGAATGACGAGCATCTGTG | 21 | CAD61188 |
| | | | | SS4R: CCCCTTAGTCGTGGATTCTTG | 22 | |
| Beta 1,3 glucanase (many | B13GG | 3.2.1.39 | starch and sucrose metabolism | B13GFG: TGTATCGGAAAGTGGTTGGC | 23 | AJ009932, |
| members of the gene family) | | | | B13GRG: CCAGGTTTCTTTGGTGTTCCAG | 24 | AY170826 |
| Beta 1,3 glucanase (class I | B13G1 | 3.2.1.39 | starch and sucrose metabolism | B13GF1: CCTTCAGCAACTCTGGAAAAC | 25 | AJ009932 |
| members of the gene family) | | | | B13GR1: CAGGTTTCTTTGGTGTTCCAGT | 26 | |
| Beta 1,3 glucanase (class II | B13G2 | 3.2.1.39 | starch and sucrose metabolism | B13GF2: CCCTGCAGCAACTTTGAAAAAT | 27 | AJ586575 |
| members of the gene family) | | | | B13GR2: CAGGTTTTTTTGGTGTTCCAGC | 28 | |
| Alpha glucan phosphorylase | AGPH | 2.4.1.11 | starch and sucrose metabolism | AGPHF: CCTGATCCTCGGTTTGAAGAG | 29 | M69038 |
| type H | | | | AGPHR: GCTCGGAAAATCATGACCAAC | 30 | |
| Alpha glucan phosphorylase | AGPHL1 | 2.4.1.11 | starch and sucrose metabolism | AGPHL1F: TGCTGACTATTTCCTTGTGGG | 31 | D00520, |
| type L1 | | | | AGPHL1R: TGCTGAACTTGTACGATCCC | 32 | X52385 |
| Alpha glucan phosphorylase | AGPHL2 | 2.4.1.11 | starch and sucrose metabolism | AGPHL2F: AGGAAACTGAGGCTGTAAAGG | 33 | X73684 |
| type L2 | | | | AGPHL2R: CTGTGGTTTATTTGGATGTGGC | 34 | |
| 1,4-alpha-glucan branching | GBE | 2.4.1.18 | starch and sucrose metabolism | GBEF: AGTTCTCATTCCTCGCATCAG | 35 | Y08786 |
| enzyme | | | | GBER: CCCTTCGTATGTGTTCTTTGG | 36 | |
| Fructose-bisphosphate | FBA | 4.1.2.13 | glycolysis and ancillary | FBAF: TTTCATCGTCCAAGCAATAACA | 37 | DQ235169 |
| aldolase-like | | | pathways | FBAR: ATTCATCAGCAGCAAGGATACC | 38 | |
| Mitochondrial ATP synthase | mATPase b | | energy metabolism, ATP | mATPbF: CAGCGACACCTCCTAAATCC | 39 | BF460265 |
| subunit beta | | | synthesis | mATPbR: TCCACGACAGCACCAATAAC | 40 | |
| Chloroplastidial ATP | cATPase a | | energy metabolism, ATP | CATPAF: GGTATCTCCGTTTCCAGAGTG | 41 | DQ386163 |
| synthase subunit alpha | | | synthesis | CATPAR: GCAAAGGCTTCTAATTCTGCG | 42 | |
| Chloroplastidial ATP | cATPase b | | energy metabolism, ATP | CATPbF: CCGTACTATCAAGAGGATTGGC | 43 | DQ231562, |
| synthase subunit beta | | | synthesis | CATPbR: ATGTTCCTCACCAACGATCC | 44 | YP_635646 |
| Basic PR1 | BPR1 | | pathogen and environmental | BPR1F: TCAAGCGGGTAAAGTATGTGG | 45 | AM907535, |
| (pathogenesis-related) | | | stresses | BPR1R: GACGTTGTCCTCTCCAGTTAC | 46 | BM113045 |
| protein (many members of the | | | | | | |
| gene family) | | | | | | |
| Osmotin (many members of the | Osml | | pathogen and environmental | OsmLF: GCCAATATAAACGGTGAATGCC | 47 | AAU95242 |
| gene family) | | | stresses | OsmLR: ATTGTTGTCCTCCGAATGTGG | 48 | |
| Elongation factor 1-α | EF1α | | translation machinery | EF1F: ATTGGAAACGGATATGCTCCA | 49 | AB061263 |
| | | | | EF1R: TGACAGGCGTTCAGGTAAGGA | 50 | |

TABLE 11
Comparison of gene expression of the 15 proteins using the qRT-PCR data and the proteomic data

| | | | | | | | Statistically |
|---|---|---|---|---|---|---|---|
| | | | | | | | significant in some time points |
| Gene name | Abrev. | EC | Function/metabolic pathway | GenBank # | Proteomic profiling | qRT-PCR | (qPCR data)-comments |
| Sucrose synthase 2 | SS2 | 2.4.1.13 | starch and sucrose | AY205084 | 1.88 ± 0.02 fold down- | 1.45 fold down-regulated 6 h | Yes, 6 h (1.45 fold down- |
| | | | metabolism | | regulated | after Confine-application; | regulated) |
| | | | | | | 1.02-3.51 up-regulated in the | |
| | | | | | | other time points | |
| Sucrose synthase 4 | SS4 | 2.4.1.13 | starch and sucrose | CAD61188 | — | Down-regulated 6 h and 24 h; | Yes, 30 min and 10 days |
| | | | metabolism | | | up-regulated 30 min, 2 h, 48 h | up-regulated; 6 h down- |
| | | | | | | and 10 days. | regulated |
| Beta 1,3 glucanase (many | B13GG | 3.2.1.39 | starch and sucrose | AJ009932, | 1.82 ± 1.67 fold up- | 1.21 to 2.18 fold up-regulated | Not significant; both S |
| members of the gene family) | | | metabolism | AY170826 | regulated | after 30 min, 6 h, 24 h, 48 h, | and T data indicate large |
| | | | | | | respectively; 1.22 and 1.24 | differences in expression |
| | | | | | | down-regulated after 2 h and 10 | among individuals. |
| | | | | | | days. | |
| Beta 1,3 glucanase (class I | B13G1 | 3.2.1.39 | starch and sucrose | AJ009932 | 1.82 ± 1.67 fold up- | Up-regulated 6 h, 24 h and 48 h; | Yes 24 h (3.36 fold up- |
| members of the gene | | | metabolism | | regulated (same data as | down-regulated 30 min, 2 h, 10 | regulated). |
| family) | | | | | above | days. | |
| Beta 1,3 glucanase (class II | B13G2 | 3.2.1.39 | starch and sucrose | AJ586575 | 1.82 ± 1.14 fold up- | 1.01 to 2.26 up-regulated in all | Yes, 24 h (2.26 fold up- |
| members of the gene | | | metabolism | | regulated | time points excepting after 30 min | regulated) |
| family) | | | | | | when it was 1.3 down- | |
| | | | | | | regulated. | |
| Alpha glucan | AGPH | 2.4.1.11 | starch and sucrose | M69038 | 2.44 ± 0.12 fold down- | 1.11-2.61 fold down-regulated | Yes, 6 h and 24 h(down- |
| phosphorylase type H | | | metabolism | | regulated | 2 h to 48 h after Confine | regulated) |
| | | | | | | application; 1.2 and 1.67 up- | |
| | | | | | | regulated after 30 min and 10 | |
| | | | | | | days, respectively | |
| Alpha glucan | AGPHL1 | 2.4.1.11 | starch and sucrose | D00520, | 1.39 ± 0.18 fold down- | 1.07-2.16 down-regulated 30 min. | No |
| phosphorylase type L1 | | | metabolism | X52385 | regulated | to 48 h; 1.13 up-regulated | |
| | | | | | | after 10 days. | |
| Alpha glucan phosphorylase | AGPHL2 | 2.4.1.11 | starch and sucrose | X73684 | — | 1.22 to 1.79 fold down- | Yes 24 h down-regulated |
| type L2 | | | metabolism | | | regulated between 2 h and | (1.79 fold) and 10 days |
| | | | | | | 48 h; 1.51 and 2.41 up-regulated | up-regulated (2.41 fold) |
| | | | | | | after 30 min. and 10 days | |
| 1,4-alpha-glucan branching | GBE | 2.4.1.18 | starch and sucrose | Y08786 | — | | |
| enzyme | | | metabolism | | | | |
| Fructose-bisphosphate | FBA | 4.1.2.13 | glycolysis and | DQ235169 (T), | Two different plastidial | Cytosolic FBA-like; | Yes, up-regulated, 30 min, |
| aldolase-like | | | ancillary pathways | CK278107 and | FBAses. | 1.05-1.64 up-regulated in all | 2 h and 24 h |
| | | | | EU344848 (S) | CK278107 | time points excepting 48 h | |
| | | | | | 1.64 ± 0.40 fold down- | when it was 1.19 fold down- | |
| | | | | | regulated; EU344848 | regulated | |
| | | | | | 1.49 ± 0.31 fold down- | | |
| | | | | | regulated. | | |
| Mitochondrial ATP synthase | mATPase b | | energy metabolism, | BF460265 | | | |
| subunit beta | | | ATP synthesis | | | | |
| Chloroplastidial ATP | cATPase a | | energy metabolism, | DQ386163 | | | |
| synthase subunit alpha | | | ATP synthesis | | | | |
| Chloroplastidial ATP | cATPase b | | energy metabolism, | DQ231562, | | | |
| synthase subunit beta | | | ATP synthesis | YP_635646 | | | |
| Basic PR1 (pathogenesis- | BPR1 | | pathogen and | AM907535, | Many different proteins. | Up-regulated 30 min to 48 h. | Yes, up-regulated after |
| related) protein (many | | | environmental | BM113045 | Up-regulated | Down-regulated after 10 days | 24 h (10.67 fold up- |
| members of the gene | | | stresses | | | | regulated) and 48 h (6.49 |
| family) | | | | | | | fold up-regulated) |
| Osmotin (many members of | Osml | | pathogen and | AAU95242 | Many different proteins. | Up-regulated 30 min, 6 h, 24 h, | Yes, up-regulated after |
| the gene family) | | | environmental | | Up-regulated | 48 h. Down regulated 2 h and 10 | 24 h (13.03 fold up- |
| | | | stresses | | | days. | regulated) and 48 h (4.04 |
| | | | | | | | fold up-regulated) |
| Elongation Factor 1-α | EF1α | | translation machinery | AB061263 (T) | 1.4 ± 0.36 fold up- | Reference gene used for all | No |
| | | | | vs CK270139 (S) = | regulated | time points, Confine | |
| | | | | DQ284495 (T) | | experiment, detached leaf | |
| | | | | 1 alpha (T) vs 1 | | experiment and infected plants | |
| | | | | alpha-like (S) | | with Phytophthora - results | |
| | | | | | | range between 1.2 up regulated | |
| | | | | | | to 1.11 down-regulated when | |
| | | | | | | compared to individual with | |
| | | | | | | the lowest expression. Seems | |
| | | | | | | to be have the most stable | |
| | | | | | | expression in various | |
| | | | | | | experimental conditions | |

TABLE 12
qPCR analysis of gene expression in plants treated with 1% Confine and
infected with P. infestans. CH—healthy leaves; CI—leaves infected with P. infestans.

| | | | | Fold |
|---|---|---|---|---|
| | | | Fold up- | down- |
| Gene | Sample type | Average and SE | regulated | regulated |
| Sucrose synthase 2 | CH | 9.79 ± 4.64 | 1.26 | |
| | CI | 12.38 ± 3.76 | | |
| Sucrose synthase 4 | CH | 2.11 ± 0.38 | 3.42 | |
| | CI | 7.20 ± 3.67 | | |
| Beta 1,3 glucanase (many | CH | 13.60 ± 6.55 | 1.18 | |
| members of the gene family) | CI | 15.99 ± 6.01 | | |
| Beta 1,3 glucanase (class I | CH | 21.57 ± 12.49 | 1.26 | |
| members of the gene family) | CI | 27.28 ± 9.33 | | |
| Beta 1,3 glucanase (class II | CH | 19.75 ± 9.76 | 1.20 | |
| members of the gene family) | CI | 23.75 ± 9.13 | | |
| Alpha glucan phosphorylase | CH | 2.48 ± 0.56 | 0.89 | 1.12 |
| type H | CI | 2.21 ± 0.53 | | |
| Alpha glucan phosphorylase | CH | 2.50 ± 0.56 | 1.1 | |
| type L1 | CI | 2.75 ± 0.82 | | |
| Alpha glucan phosphorylase | CH | 3.41 ± 0.57 | 1.05 | |
| type L2 | CI | 3.59 ± 1.37 | | |
| 1,4-alpha-glucan branching | CH | 2.07 ± 0.20 | 1.24 | |
| enzyme | CI | 2.58 ± 0.84 | | |
| Fructose-bisphosphate aldolase- | CH | 1.76 ± 0.12 | 1.56 | |
| like | CI | 2.75 ± 0.88 | | |
| Mitochondrial ATP synthase | CH | 2.15 ± 0.45 | 3.02 | |
| subunit beta | CI | 6.49 ± 3.83 | | |
| Chloroplastidial ATP synthase | CH | 3.40 ± 0.87 | 1.16 | |
| subunit alpha | CI | 3.95 ± 1.08 | | |
| Chloroplastidial ATP synthase | CH | 1.76 ± 0.27 | 1.19 | |
| subunit beta | CI | 2.10 ± 0.45 | | |
| Basic PR1 (pathogenesis- | CH | 2.22 ± 0.48 | 3.72 | |
| related) protein (many members | CI | 8.24 ± 4.18 | | |
| of the gene family) | | | | |
| Osmotin (many members of the | CH | 1.58 ± 0.35 | 2.77 | |
| gene family) | CI | 4.37 ± 1.30 | | |

### REFERENCES

Andreu A, Guevara M, Wolski E, Daleo G, Caldiz D (2006) Enhancement of natural disease resistance in potatoes by chemicals. Pest Manag. Sci. 62: 162-170
Balci Y, Balci S, Eggers J, MacDonal W L, Juzwik J, Long R P, Gottschalk K W (2007) Phytophthora spp. associated with forest soils in eastern and north-central US oak ecosystems. Plant Dis. 91: 705-710
Bevan M, Bancroft I, Bent E, Love K et al., (1998) Analysis of 1.9 Mb of contiguous sequence from chromosome 4 of Arabidopsis thaliana. Nature. 391: 485-488
Cahill D M, Rookes J E, Wilson B A, Gibson L, McDougall K L (2008) Phytophthora cinnamomi and Australia's biodiversity: impacts, predictions and progress towards control. Aus. J. Bot. 56: 279-310
Caux P-Y, Kent R, Fan G, Stephenson G (1996) Environmental fate and effects of chlorothalonil: A canadian perspective. Crit. Rev. Environ. Sci. Technol. 26: 45-93
Cohen Y, Coffey M D (1986) Systemic fungicides and the control of oomycetes. Ann. Rev. Phytopathol. 24: 311-338
Coil N S, Epple P, Dangl J L (2011) Programmed cell death in the plant immune system. Cell Death and Differentiation. 1-10
Daniel R, Guest D (2006) Defense responses induced by potassium phosphonate in Phytophthora palmivora-challenged Arabidopsis thaliana. Physiol. Mol. Plant Pathol. 67: 194-201
Daniel R, Wilson B A, Cahill D (2005) The effect of potassium phosphonate on the response of Xanthorrhoea australis to infection by Phytophthora cinnamomi. Australas. Plant Path. 34: 541-548
Dowley L, O'Sullivan E (1981) Metalaxyl-resistance in populations of Phytophthora infestans (Mont.) de Bary in Ireland. Potato Res. 24: 417-421
Durrant W, Dong X (2004) Systemic acquired resistance. Annu. Rev. Phytopathol. 42: 185-209
FRAC (2009) FRAC (Fungicide Resistance Action Committee) code list http://www.frac.info/frac/publication/anhang/FRAC_CODE_LIST.pdf
Fry W (2008) Phytophthora infestans: the plant (and R gene) destroyer. Mol. Plant Pathol. 9: 385-402
Glazebrook J (2005) Contrasting mechanisms of defense against biotrophic and necrotrophic pathogens. Annu. Rev. Phytopathol. 43: 205-227
Goodwin S, Cohen B, Deahl K, Fry W (1994) Migration from northern Mexico was the probable cause of recent genetic changes in populations of Phytophthora infestans in the United States and Canada. Phytopathol. 84: 553-558
Guest D (1984) Modification of defence responses in tobacco and Capsicum following treatment with fosetyl-Al[aluminium tris(o-etyl phosphonate)]. Physiol. Plant Pathol. 25: 125-134
Guest D (1986) Evidence from light microscopy of living tissues that Fosetyl-Al modifies the defense in tobacco seedlings following inoculation by Phytophthora nicotiana var nicotianae. Physiol. Mol. Plant Pathol. 29: 251-261
Guest D, Bompeix G (1990) The complex mode of action of phosphonates. Australas. Plant Pathol. 19: 113-115
Guest D, Grant B (1991) The complex action of phosphonates as antifungal agents. Bio. Rev. 66: 159-187
Jackson T, Burgess T, Colquhoun I, Hardy G (2000) Action of the fungicide phosphite on Eucalyptus marginata inoculated with Phytophthora cinnamomi. Plant Pathol. 49: 147-154
James C (1971b) A manual of assessment keys for plant disease. Canada Dept. Agri. Pub. No. 1458
King M, Reeve W, van der Hoek M B, Williams N, McComb J, O'Brien P A, Hardy G E S (2010) Defining the phosphite-regulated transcriptome of the plant pathogen Phytophthora cinnamomi. Mol. Genet. Genomics 284: 425-435
Kuc J (1987) Translocated signals for plant immunization. Annals New York Acad. Sci. 494: 221-223
Livak K J, Schmittgen T D (2001) Analysis of relative gene expression data using real-time quantitative PCR and the 2(-Delta Delta C(T) method. Methods. 25: 402-408
Lobato M, Olivieri F, Altamiranda E, Wolski E, Daleo G, Caldiz, Andreu A (2008) Phosphite compounds reduce disease severity in potato seed tubers and foliage. Eur. J. Plant Pathol. 122: 349-358
Matheron M, Porchas M (2000) Impact of azoxystrobin, dimethomorph, fluazinam, fosetyl-Al and metalaxyl on growth, sporulation, and zoospore cyst germination of three Phytophthora spp. Plant Dis. 84: 454-458
Mayton H, Myers K, Fry W E (2008) Potato late blight in tubers—The role of foliar phosphonate applications in suppressing pre-harvest tuber infections. Crop Protection 27: 943-950
McMahon P J, Purwantara A, Wahab A, lmron M, Lambert S, Keane P J, Guest D (2010) Phosphonate applied by truck injection controls stem canker and decreases Phytophthora pod rot (black pod) incidence in cocoa in Sulawesi. Australas. Plant Pathol. 29: 170-175
Mur L A J, Kenton P, Lloyd A J, Ougham H, Prats E (2008) The hypersensitive response; the centenary is upon us but how much do we know? J. Exp. Bot. 59: 501-520
Nemestothy G, Guest D (1990) Phytoalexin accumulation, phenylalanine ammonia lyase activity and ethylene biosynthesis in fosetyl-Al treated resistant and susceptible tobacco cultivars infected with Phytophthora nicotiana var. nicotianae. Physiol. Mol. Plant Pathol. 37: 207-219
Oostendorp M, Kunz W, Dietrich B, Staub T (2001) Induced disease resistance in plants by chemical. Eur. J. Plant Pathol. 107: 19-28
Pieterse C M J, Leon-Reyes A, van der Ent S, van Wees S C M (2009) Networking by small-molecule hormones in plant immunity. Nature Chem. Biol. 5; 308-316
Rooney H, Klooster J, van der Hoorn R, Joosten M, Jones J, Wit P (2005) Cladosporium Avr2 inhibits tomato Rcr3 protease required for Cf-2 dependent disease resistance. Science 308: 1783-1786
Ryals J, Uknes S, Ward E (1994) Systemic acquired resistance. Plant Physiol. 104: 1109-1112
Suddaby T, Alhussaen K, Daniel R, Guest D (2008) Phosphonate alters the defense responses of Lambertia species challenged by Phytophthora cinnamomi. Aus. J. Bot. 56: 550-556
Tian M, Win J, Song J, van der Hoon R, van der Knaap E, Kamoun S (2007) A phytophthora infestans cystatin-like protein targets a novel tomato papain-like apoplastic protease. Plant Physiol. 143: 364-377
Thordal-Christensen H, Zhang Z, Wei Y, Collinge D (1997) Subcellular location of H₂O₂ in plants. H₂O₂ accumulation in papillae and hypersensitive response during the barley-powdery mildew interaction. Plant J. 11: 1187-1194
Wang-Pruski G, Coffin R H, Peters R D, Al-Mughrabi K I, Platt A W, Pinto D, Veenhuis-MacNeill S, Hardy W, Lim S, Astatkie T (2010) Phosphorous acid for late blight suppression in potato leaves. Am. J. Plant Sci. and Biotech. 4: 25-29
Wendehenne D, Durner J, Chen Z, Klessig D (1998) Benzothiadiazole, an inducer of plant defenses, inhibits catalase and ascorbate peroxidase. Phytochem. 47: 651-657
Wilkinson C J, Holmes J M, Dell B, Tynan K M, McComb J A, Shearer B L, Colquhoun I J, Hardy G E S (2001) Effect of phosphite on in planta zoospore production of Phytophthora cinnamomi. Plant Pathol. 50: 587-593
Wong M, McComb J A, Hardy G E S, O'Brien P A (2009) Phosphite induces expression of a putative proteophosphoglycan gene in Phytophthora cinnamomi. Australas. Plant Pathol. 38: 235-241

1. A screening assay for identifying a substance useful in suppressing Phytophthora infection or triggering programmed cell death in a Solanaceae plant comprising
administering a test substance to a Solanaceae cell or plant; and
determining the expression level of at least one of the genes listed in Table 1A and/or Table 1B or variants or homologs thereof compared to a control;
wherein an increase in the expression level of the at least one gene listed in Table 1A or variants or homologs thereof or a decrease in the expression level of the at least one gene listed in Table 1B or variants or homologs thereof indicates that the test substance is useful in suppressing Phytophthora infection or triggering programmed cell death.

2. A method of determining whether a treatment is effective for suppressing Phytophthora infection comprising
determining the expression level of at least one of the genes listed in Table 1A and/or Table 1B or variants or homologs thereof in a Solanaceae plant treated for Phytophthora infection compared to a control in the absence of treatment;
wherein an increase in the expression level of the at least one gene listed in Table A or variants or homologs thereof or a decrease in the expression level of the at least one gene listed in Table 1B or variants or homologs thereof indicates that the treatment is effective in suppressing Phytophthora infection.

3. A method of suppressing Phytophthora infection or of triggering programmed cell death, in a member of the Solanaceae family comprising administering at least one modulator to a Solanaceae plant or cell, wherein the at least one modulator modulates at least one of the genes listed in Table 1 or variants or homologs thereof or genes encoding functionally related proteins; with the proviso that the modulator is not phosphorous acid.

4. The method of claim 3, wherein Phytophthora infection is delayed by at least 0.5 weeks, at least 1 week, at least 1.5 weeks or at least 2 weeks.

5. The method of claim 3, wherein the Phytophthora infection is Phytophthora infestans or Phytophthora erythroseptica.

6. (canceled)

7. The method of claim 3, wherein the Solanaceae plant is a potato plant or a tomato plant.

8. The method of claim 3, wherein the at least one modulator comprises at least one activator of at least one of the genes listed in Table 1A and/or at least one inhibitor of at least one of the genes listed in Table 1B.

9. The method of claim 8, wherein the at least one activator activates at least 2, 5, 10, 20, 30, 40, 50, 60, 70, 72 or 84 of the genes listed in Table 1A.

10. The method of claim 8, wherein at least 2, 5, 10, 20, 30, 40, 50, 60, 70, 72 or 84 activators of the genes listed in Table 1A are administered.

11. The method of claim 8, wherein the at least one inhibitor inhibits at least 2, 5, 10, 20, 30, 31 or 32 of the genes listed in Table 1B.

12. The method of claim 8, wherein at least 2, 5, 10, 20, 30, 31 or 32 inhibitors of the genes listed in Table 1B are administered.

13. The method of claim 3, wherein the at least one modulator modulates at least one of the genes listed in Tables 7 and/or 10.

14. The method of claim 8, wherein the at least one activator comprises an isolated nucleic acid molecule of at least one of the genes listed in Table 1A or variants or homologs thereof.

15. The method of claim 14, wherein the nucleic acid molecule comprises a recombinant expression vector.

16. The method of claim 15, wherein the recombinant expression vector is contained in a host cell.

17. The method of claim 8, wherein the at least one activator comprises a protein or variant thereof encoded by at least one of the genes listed in Table 1A or a variant or homolog thereof.

18. The method of claim 8, wherein the at least one inhibitor comprises an antisense RNA, siRNA, or shRNA of at least one of the genes listed in Table 1B or variants or homologs thereof.

19. The method of claim 8, wherein the at least one inhibitor is an aptamer that inhibits at least one of the proteins encoded by the genes listed in Table 1B or variants or homologs thereof or an antibody or antibody fragment against a protein encoded by at least one of the genes listed in Table 1B or variants or homologs thereof.

20. A composition comprising at least one activator of at least one of the genes listed in Table 1A or variants or homologs thereof or of genes encoding functionally related proteins and/or at least one inhibitor of at least one of the genes listed in Table 1B or variants or homologs thereof or of genes encoding functionally related proteins, in admixture with a suitable carrier; with the proviso that the activator or inhibitor is not phosphorous acid.

21. The composition of claim 20, wherein the at least one activator activates at least one of the genes listed in Tables 9 and/or 11 as being upregulated or variants or homologs thereof and/or the at least one inhibitor inhibits at least one of the genes listed in Tables 9 and/or 11 as being downregulated or variants or homologs thereof.

22. The composition of claim 20, wherein the at least one activator is a sense nucleic acid of at least one of the genes listed in Table 1A or variants or homologs thereof or a protein encoded by at least one of the genes listed in Table 1A or variants or homologs thereof.

23. The composition of claim 20, wherein the at least one inhibitor is an antisense nucleic acid of at least one of the genes listed in Table 1B or variants or homologs thereof or an antibody against a protein encoded by at least one of the genes listed in Table 1B or variants or homologs thereof.

24. The composition of claim 20, comprising at least one activator that activates at least 2, 5, 10, 20, 30, 40, 50, 60, 70, 72 or 84 of the genes listed in Table 1A or variants or homologs thereof and/or at least one inhibitor that inhibits at least 2, 5, 10, 20, 30, 31 or 32 of the genes listed in Table 1B or variants or homologs thereof.

25. The composition of claim 20, comprising at least 2, 5, 10, 20, 30, 40, 50, 60, 70, 72 or 84 activators of the genes listed in Table 1A or variants or homologs thereof and/or at least 2, 5, 10, 20, 30, 31 or 32 inhibitors of the genes listed in Table 1B or variants or homologs thereof.
